SEQUENCE LISTING (10 sequences)

SEQ ID NO: 1 - protein, 8 aa, Artificial
  Synthetically Generated Peptide
MSRPACPN

SEQ ID NO: 2 - protein, 7 aa, Artificial
  Synthetically Generated Peptide
AFLARAA

SEQ ID NO: 3 - protein, 12 aa, Artificial
  Synthetically Generated Peptide
RCLSSSAVANRS

SEQ ID NO: 4 - protein, 7 aa, Artificial
  Synthetically Generated Peptide
KKSRALF

SEQ ID NO: 5 - protein, 14 aa, Artificial
  Synthetically Generated Peptide
SFLLRNPNDKYEPF

SEQ ID NO: 6 - protein, 9 aa, Artificial
  Synthetically Generated Peptide
SFLLRNPND

SEQ ID NO: 7 - protein, 9 aa, Artificial
  Synthetically Generated Peptide
NPNDKYEPF

SEQ ID NO: 8 - protein, 26 aa, Artificial
  Synthetically Generated Peptide
GRAVYLNKSRFPPMPPPPFISEDASG

SEQ ID NO: 9 - protein, 6 aa, Artificial
  Synthetically Generated Peptide
FSLLRY

SEQ ID NO: 10 - protein, 6 aa, Artificial
  Synthetically Generated Peptide
LSIGRL

(19) United States
(12) Patent Application Publication
(10) Pub. No.: US 2009/0022729 A1
(43) Pub. Date: Jan. 22, 2009

(54) METHODS AND COMPOSITIONS FOR TREATING CARDIAC DYSFUNCTIONS

(76) Inventors: Nigel Mackman, Chapel Hill, NC (US); Rafal Pawlinski, Chapel Hill, NC (US); Burns C. Blaxall, Pittsford, NY (US)

Correspondence Address: CATALYST LAW GROUP, APC, 9710 SCRANTON ROAD, SUITE S-170, SAN DIEGO, CA, 92121, US

(21) Appl. No.: 12/101,638

(22) Filed: Apr. 11, 2008

Related U.S. Application Data
(60) Provisional application No. 60/923,290, filed on Apr. 13, 2007.

(51) Int. Cl. A61K 39/395; A61K 38/02; A61K 31/7088; A61K 48/00; A61K 38/05; A61K 31/426; A61K 31/17; A61K 31/4453; A61K 31/55; A61K 31/538; A61K 31/519; A61K 38/08; A61K 31/4184; A61K 38/10; C12N 5/06

(52) U.S. Cl. 424/143.1; 424/130.1; 514/2; 514/44; 514/19; 514/369; 514/597; 514/326; 514/217.1; 514/230.5; 514/267; 514/388; 514/16; 514/14; 514/17; 435/375

(57) ABSTRACT

The present invention provides methods and pharmaceutical compositions for treating or preventing cardiac dysfunctions (e.g., cardiac hypertrophy, cardiac remodeling, or heart failure) in subjects who have or are likely to develop cardiomyopathies. Some of the methods are directed to therapeutic or prophylactic treatment of cardiac dysfunctions in subjects having undergone myocardial injuries such as cardiac ischemia/reperfusion or myocardial infarction. Typically, these methods comprising administering to the subjects a therapeutic composition comprising a compound which can specifically inhibit PAR1 mediated signaling or down-regulate the cellular level of PAR1.

### CROSS-REFERENCES TO RELATED APPLICATIONS

This application claims priority from U.S. Provisional Application Ser. No. 60/923,290 by Mackman et al., entitled “Methods and Compositions for Treating Cardiac Dysfunctions,” filed on Apr. 13, 2007, the contents of which are incorporated herein in their entirety by this reference.

### STATEMENT REGARDING FEDERALLY SPONSORED RESEARCH OR DEVELOPMENT

This invention was made in part by government support from the National Institutes of Health, Grant No. HL71053. The United States government therefore may have certain rights in the invention.

### BACKGROUND OF THE INVENTION

This invention is directed to methods and compositions for the treatment of cardiac dysfunctions, particularly cardiomyopathies. The methods and compositions are based on the discovery that the G-protein-coupled receptor PAR1 is associated with pathological signaling that leads to cardiomyopathy and other cardiac dysfunctions.

Cardiomyopathies are disorders caused by or associated with myocardial dysfunctions. A common complication of all of the cardiomyopathies is progressive congestive heart failure. For example, insufficient blood supply to the myocardium can result in myocardial injury such as ischemia and infarction. Myocardial ischemia is a condition in which oxygen deprivation to the heart muscle is accompanied by inadequate removal of metabolites because of reduced blood flow or perfusion. Myocardial infarction is the necrosis of the myocardial tissue due to an occluded blood supply to the heart muscles. When the blood supply is blocked, the tissue normally supplied with blood by the blocked artery becomes ischemic and die off (necrosis). The heart responds to infarction by hypertrophy of surviving cardiac muscle in an attempt to maintain normal contraction. However, when the hypertrophy is insufficient to compensate, cardiac remodeling and reduced cardiac function result, leading to heart failure and death.

Protease activated receptor 1 (PAR1) is a G-protein-coupled receptor activated via proteolytic cleavage by various proteases, including the coagulation protease thrombin. Once cleaved, PAR1 rapidly transmits a signal across the plasma membrane to internally located G proteins, and ultimately leads to platelet aggregation. Stimulation of G proteins by activated PAR1 also causes a rapid rise in intracellular calcium and activation of the GP IIb/IIIa fibrinogen receptor. PAR1 is expressed in various tissues, e.g., endothelial cells, smooth muscle cells, fibroblasts, neurons and human (but not mouse) platelets. 50% of PAR1^{-/-} embryos survive development and adult PAR1^{-/-} mice have no phenotypic abnormalities in any tissue. In the heart, PAR1 is expressed by cardiomyocytes and cardiac fibroblasts.

More specifically, myocardial infarction induces structural remodeling of the heart, in which areas of initial infarct are replaced with collagen-rich tissue (1,2) These changes induce cardiac remodeling and hypertrophy, which are associated with reprogramming of cardiac gene expression and induction of the “fetal gene program.” For instance, the fetal genes atrial natriuretic factor (ANF) and B-type natriuretic peptide (BNP) are upregulated as a compensatory mechanism to promote natriuresis and to suppress myocyte hypertrophy (3). Importantly, the MAPK (mitogen-activated protein kinase)/ERK (extracellular signal-regulated kinase) kinase (MEK1)-ERK1/2 pathway has been shown to protect the myocardium from ischemia/reperfusion (I/R) injury (4,5).

It has been shown that that myocardial infarction induces damage to the endothelial barrier that allows leakage of coagulation factors into the myocardium and their activation by tissue factor (TF) expressed by cardiomyocytes (6) Importantly, inhibition of either TF or thrombin significantly reduced the infarct size in rabbit models of cardiac I/R injury (6,7) A recent study showed that the fibrin degradation fragment E1 contributes to inflammation after myocardial infarction by binding to vascular endothelial cadherin at cell-cell junctions in the endothelium and facilitating the recruitment of neutrophils into the myocardium (8).

Members of the protease-activated receptor (PAR) family of G-protein-coupled receptors are activated by proteolytic cleavage (9). PAR-1 is expressed by a variety of cell types and is activated by the coagulation protease thrombin, as well as other proteases (9,10). Cleavage of PAR-1 results in activation of Gαq, Gα12/13, and Gαi, as well as downstream signaling pathways, including the MAPK pathways, ERK 1/2 and ERK5 (9,11). PAR-1 plays a critical role in the activation of human platelets but is not expressed on mouse platelets (9). Despite the death of ≈50% of PAR1^{-/-} embryos at mid-gestation, adult PAR1^{-/-} mice have no phenotypic abnormalities in any tissue (12,13).

In the heart, PAR-1 is expressed by cardiomyocytes and cardiac fibroblasts (14,15). A recent study showed that PAR-1 expression was increased in the hearts of patients with ischemic and idiopathic dilated cardiomyopathy (16). In addition, PAR-1 expression is increased in the left ventricle (LV) in a mouse model of chronic heart failure (17) In vitro studies using rat neonatal cardiomyocytes demonstrated that activation of PAR-1 induced hypertrophy (14,18). PAR-1-dependent changes included increases in intracellular calcium, protein content and cell size, sarcomeric organization, and ANF expression. Furthermore, activation of PAR-1 on cardiac fibroblasts induces cell proliferation (15). These results strongly suggest that PAR-1 may contribute to cardiac remodeling after injury (19).

Although there are many treatments for some of the consequences of cardiomyopathy, cardiac remodeling, and heart failure, there is no treatment that acts directly on the biological mechanism that induces cardiomyopathy, cardiac remodeling, and heart failure. Accordingly, there is a need for better pharmaceutical agents and methods for the treatment of cardiomyopathy, cardiac remodeling, and heart failure that act directly on the biological mechanisms causing these pathological changes. Preferably, these agents and methods are well tolerated, do not cause side effects, and can be used together with other treatments for the consequences of cardiomyopathy, cardiac remodeling, and heart failure.

### SUMMARY OF THE INVENTION

In one aspect, the invention provides methods for treating or preventing cardiac dysfunction in a subject having or being at risk of developing a cardiomyopathy. The methods entail administering to the subject a therapeutically effective amount of an antagonist of protease activated receptor 1 (PAR1), thereby treating or preventing cardiac dysfunction in the subject. Examples of cardiac dysfunctions to be treated or prevented in the subject include cardiac hypertrophy, cardiac remodeling, and heart failure. Some of the methods are directed to treating subjects who have are at risk of developing an extrinsic cardiomyopathy, e.g., ischemic cardiomyopathy. For example, the subject can be one who has undergone a myocardial injury such as myocardial infarction or cardiac ischemia/reperfusion. Some other methods of the invention are directed to treating subjects who have or are at risk of developing an intrinsic cardiomyopathy, e.g., dilated cardiomyopathy.

In some preferred methods, the PAR1 antagonist to be administered is a PAR1 selective antagonist. The PAR1 antagonist that can be used in the methods can be a peptide, a peptide mimetic, a small molecule organic compound, a pepducin, a polynucleotide or an antibody. In some methods, the administered PAR1 antagonist inhibits a PAR1 signaling activity. Some of these methods employ a PAR1 antagonist which is a peptidomimetic, e.g., RWJ-56110 or (αS)—N-[(1S)-3-amino-1-[[(phenylmethyl)amino]carbonyl]propyl]-α-[[[[[1-(2,6-dichlorophenyl)methyl]-3-(1-pyrrolidinylmethyl)-1H-indol-6-yl]amino]carbonyl]amino]-3,4-difluorobenzenepropanamide. Some other methods employ a PAR1 antagonist which is a small molecule organic compound, e.g., SCH-79797, which is N-3-cyclopropyl-7-{[4-(1-methylethyl)phenyl]methyl}-7H-pyrrolo[3,2-f]quinazoline-1,3-diamine. In some other methods of the invention, the administered PAR1 antagonist down-regulates cellular level of PAR1. For example, the PAR1 antagonist to be employed in these methods can be a siRNA. In some of the methods, the PAR1 antagonist is delivered locally to the heart of the subject. Some of the methods further involve administering to the subject a second therapeutic agent for cardiac dysfunction.

In a related aspect, the invention provides methods for treating or preventing cardiac dysfunction in a subject who has undergone myocardial injury. The methods entail administering to the subject a therapeutically effective amount of an antagonist of protease activated receptor 1 (PAR1). The myocardial injury which the subject has undergone can be, e.g., myocardial infarction or cardiac ischemia/reperfusion. The cardiac dysfunctions to be treated or prevented in these subjects include, e.g., cardiac remodeling, cardiac hypertrophy, and heart failure. In some preferred embodiments, the PAR1 antagonist to be administered to the subject is a PAR1 selective antagonist. The PAR1 antagonist used in these methods can inhibit a PAR1 signaling activity or down-regulate cellular level of PAR1. The PAR1 antagonist can be a peptide, a peptide mimetic, a small molecule organic compound, a pepducin, a polynucleotide or an antibody.

In another aspect, the invention provides methods for treating or preventing hypertrophy in a cardiomyocyte cell or proliferation of a cardiac fibroblast. Such methods entail contacting the cardiomyocyte cell or cardiac fibroblast with a PAR1 antagonist. In some of these methods, the cardiomyocyte cell or cardiac fibroblast is present in a subject having undergone myocardial injury such as myocardial infarction or cardiac ischemia/reperfusion. Preferably, the PAR1 antagonist employed in the methods is a PAR1 selective antagonist. The PAR1 antagonist can inhibit a PAR1 signaling activity or down-regulate cellular level of PAR1. It can be a peptide, a peptide mimetic, a small molecule organic compound, a pepducin, a polynucleotide or an antibody.

The invention also provides methods of administering a PAR1 antagonist based therapeutic composition to subjects who are concurrently receiving treatment with other known medications for cardiac dysfunctions. Subjects suitable for treatment with PAR1 antagonist based therapy also include patients who had or will have surgical procedures for cardiac dysfunction such as heart failure, as well as patients who have implantable cardiac devices such as ventricular pacemakers or cardioverter defibrillators.

Specifically, one aspect of the invention is a method for treating or preventing cardiac dysfunction in a subject having or being at risk of developing a cardiomyopathy comprising administering to a subject a therapeutically effective amount of an antagonist of protease activated receptor 1 (PAR1), thereby treating or preventing cardiac dysfunction in the subject, wherein the subject:

(1) has or is at risk of developing a cardiomyopathy; or

(2) has undergone a myocardial injury.

Yet another aspect of the invention is a method for treating or preventing hypertrophy in a cardiomyocyte cell or proliferation of a cardiac fibroblast comprising contacting the cell with an antagonist of protease activated receptor 1 (PAR1), thereby treating or preventing hypertrophy in the cardiomyocyte cell.

Yet another aspect of the invention is a pharmaceutical composition for treating or preventing cardiac dysfunction comprising

(1) a therapeutically effective dose of a PAR1 antagonist; and

(2) a pharmaceutically acceptable carrier, diluent or excipient in unit dosage form.

A further understanding of the nature and advantages of the present invention may be realized by reference to the remaining portions of the specification and claims.

### BRIEF DESCRIPTION OF THE DRAWINGS

The following invention will become better understood with reference to the specification, appended claims, and accompanying drawings, where:

FIGS. 1A-1B show the role of PAR1 in infarct size and remodeling after cardiac I/R injury. (A) Determination of area at-risk (AAR) and infarct size in mice subjected to 30 minutes of ischemia and 2 hours of reperfusion. I/R-injured WT mice were treated with either saline (n=8) (white bars) or hirudin (n=5) (stippled bars). Infarct size was measured in PAR1^{-/-} mice (n=14) (black bars) and WT littermates (n=10) (white bars). *P<0.05. (B) Echocardiographic analysis of LV diameter (LVD) at diastole (d) and at systole (s), LV fractional shortening (FS) and mean velocity of circumferential fiber shortening (mVcf) before and after myocardial infarction induced by 45 minutes of ischemia and 2 weeks of reperfusion. Results from WT (n=7) (white bars) and PAR1^{-/-} (n=7) (black bars) mice are shown. *P<0.05.

FIGS. 2A-2B show PAR1 expression in hypertrophic and failing human hearts. (A) Levels of PAR1 protein in non-hypertrophic (n=3) or hypertrophic (n=3) human hearts are shown. Representative western blots are shown above. Data are shown as mean±SEM. *P<0.05. (B) Real time PCR analysis of PAR1 mRNA expression in heart samples from humans with heart failure (HF) (n=9) and non-failing (NF) donors (n=5).

FIGS. 3A-3D show PAR1 expression in mice overexpressing PAR1 in cardiomyocytes. (A) PAR1 mRNA expression was analyzed by northern blotting in various tissues from transgenic (αMHC-PAR1) (line 18) and non-transgenic (WT) mice. 10 μg of total RNA was analyzed in each lane. PAR1 mRNA expressed by the transgene is smaller than PAR1 mRNA from the endogenous PAR1 gene because it lacks most of the 3′ untranslated region. The larger bands may represent partially spliced transcripts. (B) Comparison of PAR1 mRNA expression in the hearts from transgenic lines 18 and 43. GAPDH was used as a loading control. Levels of PAR1 protein expression in various mice were analyzed by western blotting (C) and immunohistochemistry (D).

FIGS. 4A-4E show characterization of mice overexpressing PAR1 in cardiomyocytes. (A) Comparison of the size of hearts from 10 month old WT and αMHC-PAR1 mice. (B) HW:BW ratio was determined in 4, 6 and 10 month (mo) old WT mice and αMHC-PAR1 mice (5-10 mice per group). (C) Northern blot analysis of ANF and BNP mRNA expression in the LV of 6 month old WT and αMHC-PAR1 mice (5 mice per group). Levels of ANF and BNP mRNA normalized to GAPDH are shown (right panel). (D) Representative cross-sections of hearts from 6 month old WT and αMHC-PAR1 mice cut at the level of the papillary muscle (left panel). Quantitation of the lumenal area of the LV and thickness of the LV free wall in WT (n=6) and αMHC-PAR1 (n=6) mice (right panel). (E) Cross-sectional area of cardiomyocytes in hearts of WT and αMHC-PAR1 (line 18) mice. Six mice were analyzed for each line and 10-12 cardiomyocytes were measured per mouse. In panels B-E, results from WT mice (white bars) and αMHC-PAR1 mice (black bars) are shown. *P<0.05.

FIGS. 5A-5C show echocardiographic analysis of heart size and function in αMHC-PAR1 mice. (A) Representative M-mode echocardiographic images of an αMHC-PAR1 mouse and a WT littermate control at 10 months of age. (B) LV diameter (LVD), anterior and posterior LV wall and LV function [percentage of fractional shortening (% FS) and mean velocity of circumferential fiber shortening (mVcf)] were measured at diastole (d) and systole (s) by echocardiography in 10 month old mice. WT (white bars) and αMHC-PAR1 (black bars) mice are shown (5 mice per group). (C) LVD and LV function (% FS and mVcf) were measured at diastole (d) and systole (s) in PAR1^{+/-} (n=5), αMHC-PAR1/PAR1^{+/-} (n=5), PAR1^{-/-} (n=7) and αMHC-PAR1/PAR1^{-/-} (n=7) mice at 10 months of age. *P<0.05.

FIGS. 6A-6D show that deletion of the tissue factor (TF) gene in cardiomyocytes reduces cardiac hypertrophy in αMHC-PAR1 mice. Data is shown for hearts of male mice (4 months of age) on a mixed genetic background (75% C57BI/6 and 25% Sv129). αMHC-PAR1 mice on this genetic background developed hypertrophy faster than αMHC-PAR1 mice on a C57BI/6 genetic background. n-number of mice in each group. (A) HW:BW ratio. (B) Levels of ANF mRNA expression in the different groups of mice were normalized to the levels of GAPDH mRNA. The northern blot above shows 3 representative mice from each group. (C) Measurement of the lumenal area of the LV and the thickness of the LV free wall. Representative cross-sections of the hearts cut at the level of the papillary muscle from αMHC-PAR1/TF^{flox/flox} mice with or without the αMHC-Cre transgene are shown. Quantitation is shown below. (D) Northern blot analysis shows no difference in the levels of PAR1 mRNA expression in the hearts of αMHC-PAR1/TF^{flox/flox}/αMHC-Cre mice and αMHC-PAR1/TF^{flox/flox} littermate controls. *P<0.05.

FIG. 7 shows role of TF, coagulation proteases and PAR1 in cardiac remodeling. I/R injury induces damage to the endothelium that allows coagulation factors to enter the myocardium where they are activated by TF expressed by cardiomyocytes and cardiac fibroblasts. Subsequent activation of PAR1 on cardiomyocytes induces hypertrophy, whereas activation of PAR1 on cardiac fibroblasts induces proliferation.

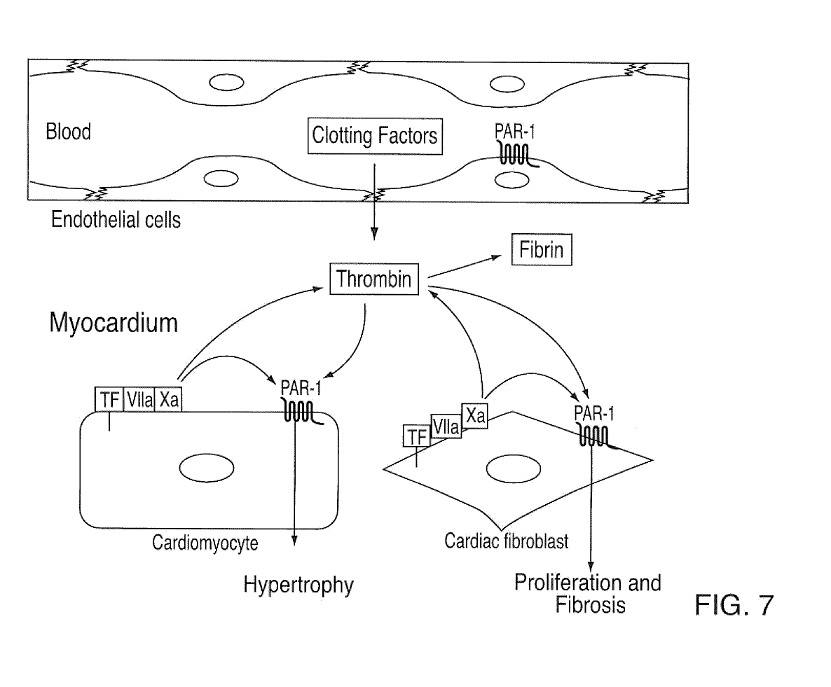

FIG. 8 shows the results of Example 8. The top panel shows ratio of the area of risk (AAR) versus the left ventricle (LV). Error bars show the standard deviation (SD). The left bar shows the results for wild-type (PAR2^{+/+}) mice, while the right bar shows the results for knockout (PAR2^{-/-}) mice. The bottom panel shows the ratio of the area of the infarct (INF) versus the area of risk. Again, the bar shows the results for wild-type (PAR2^{+/+}) mice, while the right bar shows the results for knockout (PAR2^{-/-}) mice.

### DETAILED DESCRIPTION OF THE INVENTION

### I. Overview

A large number of biological functions are mediated through the activity of G-protein-coupled receptors (GPCR). Accordingly, many disease processes and conditions are related to the activities of these receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCRs are summarized in S. Watson & S. Arkinstall, “The G-Protein Linked Receptor Facts Book” (Academic Press, London, 1994), incorporated herein by this reference.

One unusual group of GPCRs is the protein activated receptors (PARs). The protease activated receptors (PARs) are a family of G-protein coupled receptors that are activated by proteolytic cleavage. Thus far, there are four known family members: PAR1, PAR2, PAR3 and PAR4. PAR1 is widely expressed. This prototype protease activated receptor was identified while attempting to understand how the coagulation protease thrombin stimulates cells. Other proteases can also proteolytically activate PAR1, including trypsin, granzyme A, and factor Xa (FXa). The other PARs can also be activated by multiple proteases. Given the number of new trypsin-like serine proteases identified, it is likely that more activating proteases for these receptors will be found. The protease α-thrombin recognizes PAR1 by binding to the receptors negatively charged domain, which is homologous to the hirudin C-terminus. This domain occupies P9′-P14′ as a substrate and is recognized by thrombin's anion binding exosite 1. PAR1 cleavage is allosterically enhanced by the docked P9′-P14′ hirudin-like domain. It has been suggested that the P9′-P14′ hirudin-like domain in the activated receptor might sequester thrombin after receptor cleavage.

Thrombin activates PAR1 by limited proteolytic cleavage between PAR1 Arg41 and Ser42 thereby generating a new N-terminus, which through intramolecular interaction, activates the receptor. Activation of this receptor can be mimicked by SFLLR (SEQ ID NO: 1) and related peptides that are homologous to the newly-generated N-terminus (PAR1 residues 42-46). These peptides can also activate the receptor directly, independently of thrombin cleavage. PAR1 domain substitution studies suggest that the ligand recognition pocket is contained in extracellular N-terminus domain PAR1 residues 76-93 and the second extracellular loop-2 (ECL-2) region (PAR1 residues 244-268). These receptor domains serve as contact points for amino acids 4 and 5 of peptide-ligand agonists. Point mutation studies indicate that Phe97 in the amino terminus and Glu260 in the receptor extracellular loop-2 confer peptide ligand selectivity, and complementary mutation studies indicate that the activating peptide Arg5 interacts with the receptor Glu260 in the receptor ECL-2. The peptide comprising the “tethered ligand” for PAR-1 (SFLLR) (SEQ ID NO: 1) can activate PAR2. Recently “transactivation” of PARs has been demonstrated in human vascular endothelial cells. Cleavage of PAR1 caused activation of PAR1 in the presence of an occluding PAR1 antagonist (BMS-200261), Presumably the “tethered ligand” of PAR1 is capable of binding to and activating PAR2 on the surface of the endothelial cells. It is not clear how prevalent transactivation of these G-coupled receptors occurs in vivo; however, it does suggest that these receptors are in close proximity on the cell surface. PAR1, when overexpressed in baculovirus-infected insect cells, self associates, and the association is maintained in the presence of SDS. One wonders if receptor association and activation are common. After proteolytic cleavage and intramolecular reorganization, PAR1 then transduces the signal to intracellular GTP-binding proteins. Gq/11, Gi2, Go, G12/13 families are activated and linked to subsequent downstream intracellular signaling molecules in many cell types. In this context, this signaling is associated with the pathology of cardiomyopathy, cardiac hypertrophy, and heart failure, particularly subsequent to a cardiac infarction (heart attack).

The present invention is predicated in part on the discovery by the present inventors that PAR1 deficiency significantly reduced cardiac remodeling and heart failure, and that increased PAR1 levels are associated with cardiac hypertrophy and heart failure. Prior to the present invention, the role of PAR1 in pathologic cardiac remodeling or heart failure was not established. As detailed in the Examples below, the inventors employed PAR1^{-/-} mice to directly examine the role of PAR1 in cardiac remodeling. Echocardiographic analysis of hearts showed that PAR1^{-/-} mice had reduced dilation of the left ventricle (LV) and reduced impairment of LV function compared to wild type littermates after ischemia-reperfusion injury. These data clearly indicate that PAR1 antagonists could lead to reduction of cardiac remodeling and hypertrophy after myocardial infarction.

The inventors also observed via Western blot and real time PCR analysis that PAR1 expression was significantly increased in hypertrophic and failing human hearts. The inventors further analyzed the effect of cardiomyocyte specific overexpression of PAR1 in mice. Histologic and echocardiographic analyses of the hearts of these mice (αMHC-PAR1) indicated that PAR1 overexpression induced eccentric hypertrophy (increased LV dimension and normal LV wall thickness) and dilated cardiomyopathy. Moreover, the role of locally generated coagulation proteases in PAR1-dependent hypertrophy was determined by deleting the tissue factor (TF) gene in cardiomyocytes. αMHC-PAR1 mice with reduced TF expression in their hearts exhibited significantly less hypertrophy. Taken together, these in vivo studies demonstrated for the first time that activation of PAR1 by coagulation proteases contributes to cardiac remodeling and hypertrophy, and that inhibition of PAR1 could provide a novel therapy to reduce hypertrophy and heart failure.

In accordance with these discoveries, the present invention provides methods for treating or preventing cardiac dysfunctions induced by or associated with various cardiomyopathies. Subjects suitable for treatment with methods of the invention include ones who have or are at risk of developing any of the extrinsic cardiomyopathies (e.g., ischemic cardiomyopathy) or intrinsic cardiomyopathies (e.g., dilated cardiomyopathy) described herein. Some of the methods are directed to treating or preventing the development of cardiac remodeling, hypertrophy or heart failure in the subject. In some methods, the subjects to be treated are those who have undergone acute myocardial injuries, e.g., myocardial infarction or cardiac ischemia/reperfusion. Some other methods of the invention are directed to treating or preventing hypertrophy of cardiomyocytes or proliferation of cardiac fibroblasts. Typically, the methods employ a PAR1 antagonist compound which is capable of specifically inhibiting PAR1 signaling activity and/or down-regulating PAR1 expression or cellular level. The following sections provide more detailed guidance for practicing the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The heart is the center of a person's circulatory system. It includes an electro-mechanical system performing two major pumping functions. The left portions of the heart, including the left atrium (LA) and the left ventricle (LV), draw oxygenated blood from the lungs and pump it to the organs of the body to provide the organs with their metabolic needs for oxygen. The right portions of the heart, including the right atrium (RA) and the right ventricle (RV), draw deoxygenated blood from the body organs and pump it to the lungs where the blood gets oxygenated. These pumping functions result from contractions of the myocardium. In a normal heart, the sinoatrial node, the heart's natural pacemaker, generates electrical impulses that propagate through an electrical conduction system to various regions of the heart to excite the myocardial tissues of these regions. Coordinated delays in the propagation of the electrical impulses in a normal electrical conduction system cause the various portions of the heart to contract in synchrony to result in efficient pumping functions. A blocked or otherwise abnormal electrical conduction and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic needs is known as heart failure.

The term “cardiac dysfunction” refers to a pathological decline in cardiac performance. Cardiac dysfunction may be manifested through one or more parameters or indicia including changes to stroke volume, ejection fraction, end diastolic fraction, stroke work, arterial elastance (defined as the ratio of left ventricular (LV) end-systolic pressure and stroke volume), or an increase in heart weight to body weight ratio. Unless otherwise noted, cardiac dysfunctions encompass any cardiac disorders or aberrant conditions that are associated with or induced by the various cardiomyopathies, cardiomyocyte hypertrophy, cardiac fibrosis, or other cardiac injuries described herein. Specific examples of cardiac dysfunction include cardiac remodeling, cardiac hypertrophy, and heart failure.

Cardiomyopathy is the deterioration of the function of the myocardium (i.e., the actual heart muscle) for any reason. People with cardiomyopathy are often at risk of arrhythmia and/or sudden cardiac death. Cardiomyopathies can generally be categorized into extrinsic cardiomyopathies and intrinsic cardiomyopathies. Extrinsic cardiomyopathies are cardiac disorders where the primary pathology is outside the myocardium itself. Most cardiomyopathies are extrinsic as the underlying myocardial injury is due to extrinsic factors such as ischemia. Examples of extrinsic cardiomyopathies include ischemic cardiomyopathy and cardiomyopathy due to systemic diseases. Ischemic cardiomyopathy is a weakness in the muscle of the heart due to inadequate oxygen delivery to the myocardium with coronary artery disease being the most common cause. Intrinsic cardiomyopathies are cardiac disorders where weakness in the muscle of the heart is not due to an identifiable external cause. Intrinsic cardiomyopathies include dilated cardiomyopathy (DCM), hypertrophic cardiomyopathy (HCM or HOCM), arrhythmogenic right ventricular cardiomyopathy (ARVC), and restrictive cardiomyopathy (RCM). Unless otherwise noted, the term cardiomyopathy also encompasses viral cardiomyopathy and postpartum cardiomyopathy.

“Myocardial injury” means injury to the muscular tissue of the heart. It may arise from myocardial infarction, cardiac ischemia/reperfusion, cardiotoxic compounds, or other causes. Myocardial injury may be either an acute or nonacute injury in terms of clinical pathology. In any case it involves damage to cardiac tissue and typically results in a structural or compensatory response. Unless otherwise noted, myocardial injury as used herein primarily refers to acute myocardial injury such as acute myocardial infarction (heart attack) and cardiac ischemia/reperfusion.

Acute myocardial infarction (AMI or Ml), commonly known as a heart attack, is a disease state that occurs when the blood supply to a part of the heart is interrupted. The resulting ischemia or oxygen shortage causes damage and potential death of heart tissue.

Ischemia is a restriction in blood supply, generally due to factors in the blood vessels, with resultant damage or dysfunction of tissue (e.g., cardiac tissue). Reperfusion injury refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

The term “cardiac remodeling” generally refers to the compensatory or pathological response following myocardial injury. Cardiac remodeling is viewed as a key determinant of the clinical outcome in heart disorders. It is characterized by a structural rearrangement of the cardiac chamber wall that involves cardiomyocyte hypertrophy, fibroblast proliferation, and increased deposition of extracellular matrix (ECM) proteins.

Cardiac fibrosis refers to an abnormal thickening of the heart valves due to inappropriate proliferation of cardiac fibroblasts. Cardiac fibrosis is a major aspect of the pathology typically seen in the failing heart. The proliferation of interstitial fibroblasts and increased deposition of extracellular matrix components results in myocardial stiffness and diastolic dysfunction, which ultimately leads to heart failure.

Organ hypertrophy is the increase of the size of an organ or in a select area of the tissue (e.g., heart or skeletal muscles). It should be distinguished from hyperplasia which occurs due to cell division increasing the number of cells while their size stays the same; hypertrophy occurs due to an increase in the size of cells, while the number of cells stays the same. Heart hypertrophy is the increase in size of the ventricle chambers of the heart. Changes can be beneficial or healthy if they occur in response to aerobic or anaerobic exercise, but ventricular hypertrophy is generally associated with pathological changes due to high blood pressure or other disease states. Although ventricular hypertrophy may occur in either the left or right or both ventricles of the heart, left ventricular hypertrophy (LVH) is more commonly encountered.

The term “contacting” has its normal meaning and refers to combining two or more agents (e.g., polypeptides, polynucleotides, other polymers, or small molecule compounds) or combining agents and cells. Contacting can occur in vitro, e.g., combining two or more agents or combining an agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate. Contacting can also occur inside the body of a subject, e.g., by administering to the subject an agent which then interacts with the intended target (e.g., a tissue or a cell).

The term “subject” for purposes of treatment refers to any animal classified as a mammal, e.g., human and non-human mammals. Examples of non-human animals include dogs, cats, cattle, horses, sheep, pigs, goats, rabbits, and other socially or economically important animals. Except when noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., a cardiac dysfunction), alleviating the symptoms or arresting or inhibiting further development of the disease, condition, or disorder. Subjects in need of treatment include patients already suffering from the disease or disorder as well as those prone to have the disorder or those in whom the disorder is to be prevented. As used herein, the terms “treating,” “alleviating,” or similar terminology do not imply a cure for heart failure, cardiomyopathy, cardiac hypertrophy or any other disease or condition, rather, this terminology is used to refer to any clinically detectable improvement in the disease or condition being treated or alleviated, including, but not limited to, improvement in stroke volume, ejection fraction, end diastolic fraction, stroke work, or arterial elastance, a decrease in heart weight to body weight ratio, improvement in subjective well-being experienced by the patient, or any other clinically detectable improvement, such as, but not limited to, reduction in fatigue, increase in physical strength, or other detectable variable. As used herein, the term “therapeutically effective amount” refers to the amount of a therapy (e.g., a prophylactic or therapeutic agent) which is sufficient to reduce or ameliorate the severity and/or duration of a cardiovascular disease or condition or one or more symptoms thereof, prevent the advancement of a cardiovascular disease or condition, cause regression of a cardiovascular disease or condition, prevent the recurrence, development, or onset of one or more symptoms associated with a cardiovascular disease or condition, or enhance or improve the prophylactic or therapeutic effect(s) of another therapy (e.g., prophylactic or therapeutic agent).

Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of cardiac remodeling and/or heart failure, a therapeutic agent may directly decrease the pathology of the disease, or render the disease more susceptible to treatment by other therapeutic agents.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analogue” is used herein to refer to a molecule that structurally resembles a reference molecule but which has been modified in a targeted and controlled manner, by replacing a specific substituent of the reference molecule with an alternate substituent. Compared to the reference molecule, an analogue would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

Administration “in combination with” one or more further therapeutic agents includes simultaneous (concurrent) and consecutive administration in any order.

The phrase “PAR1 signaling activity” refers to one or more of the biochemical reactions and cellular responses induced by the interaction of a PAR1 receptor on a PAR1-expressing cell with a PAR1 stimulatory compound or agent (e.g., thrombin). These include, e.g., cleavage of PAR1 by a protease such as thrombin, activation of PAR1 by the exposed tethered ligand, activation of G proteins by PAR1, increase of intracellular calcium, activation of the GP IIb/IIIa (IIbβ3) fibrinogen receptor, and formation of platelet aggregate.

As used herein, the term “nucleic acid,” “nucleic acid sequence,” “polynucleotide,” or similar terms, refers to a deoxyribonucleotide or ribonucleotide oligonucleotide or polynucleotide, including single- or double-stranded forms, and coding or non-coding (e.g., “antisense”) forms. The term encompasses nucleic acids containing known analogues of natural nucleotides. The term also encompasses nucleic acids including modified or substituted bases as long as the modified or substituted bases interfere neither with the Watson-Crick binding of complementary nucleotides or with the binding of the nucleotide sequence by proteins that bind specifically, such as zinc finger proteins. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl)glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). Bases included in nucleic acids include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N⁶-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N⁶-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N⁶-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. DNA may be in the form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. Watson et al. Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) structure and/or activity, e.g., antibody activity, enzymatic activity, or receptor activity. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations” when the three-dimensional structure and the function of the protein to be delivered are conserved by such a variation.

Additionally, some peptides and peptidomimetics suitable for use in methods and compositions incorporate amino acids other than the standard 20 amino acids typically incorporated into proteins by the translation process; i.e., amino acids that are linked to transfer RNA (tRNA) molecules that bind to triplets in the mRNA by base-pairing. These other amino acids can either be derivatives of one of the standard 20 amino acids, amino acids in the D-configuration (i.e., optical isomers of the amino acids typically incorporated into proteins), or other amino acids, either naturally occurring amino acids that do not occur naturally in proteins, or synthetic, non-naturally-occurring amino acids. Among these amino acids are the following, with the abbreviations used herein: 2-aminoadipic acid (Aad); 3-aminoadipic acid (bAad); β-alanine (bAla); 2-aminobutyric acid (Abu); 4-aminobutyric acid (4Abu); 6-aminocaproic acid (Acp); 2-aminoheptanoic acid (Ahe); 2-aminoisobutyric acid (Aib); 3-aminoisobutyric acid (bAib); 2-aminopimelic acid (Apm); 3-aminopropionic acid; 3-cyclohexylalanine (Cha); 2,4-diaminobutyric acid (Dbu); desmosine (Des); 2,2′-diaminopimelic acid (Dpm); 2,3-diaminopropanoic acid (Dpr); Ne-ethyglycine (EtGly); N-ethylasparagine (EtAsn); hydroxylysine (Hyl); allo-Hydroxylysine (aHyl); 3-hydroxyproline (3Hyp); 4-hydroxyproline (4Hyp); isodesmosine (Ide); allo-isoleucine (alle); N-methylglycine or sarcosine (MeGly or Sar); N-methylisoleucine (Melle); 6-N-methylysine (MeLys); N-methylvaline (MeVal); norvaline (Nva); norleucine (Nle); citrulline; homocitrulline; t-butylglycine; t-butylalanine; phenylglycine; 4-fluorophenylalanine; homoarginine; 3-(2-naphthyl)alanine; 4-methoxyphenylalanine; 4-guanidinophenylalanine; benzylhistidine; and ornithine (Orn). Analogues and derivatives of these amino acids can also be used. In addition, the resulting peptides and peptide portions of peptidomimetics can be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure and Molecular Properties, 2nd ed., T. E. Creighton, W. H. Freeman and Company, New York (1993); Post-Translational Covalent Modification of Proteins, B. C. Johnson, Ed., Academic Press, New York, pgs. 1-12 (1983); Seifter et al., Meth. Enzymol. 182:626-646 (1990), and Rattan et al., Ann. N.Y. Acad. Sci. 663:30-62 (1992).

As used herein, the term “isolated” with reference to a nucleic acid molecule or polypeptide or other biomolecule means that the nucleic acid or polypeptide has been separated from the genetic environment from which the polypeptide or nucleic acid were obtained. It may also mean that the biomolecule has been altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” is meant that the nucleic acid is free of the coding sequences of those genes that, in a naturally-occurring genome immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

“Isolated” or “purified” as those terms are used to refer to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the protein of interest can be present at various degrees of purity in these preparations. Particularly for proteins, the procedures may include for example, but are not limited to, ammonium sulfate fractionation, gel filtration, ion exchange change chromatography, affinity chromatography, density gradient centrifugation, electrofocusing, chromatofocusing, and electrophoresis.

A preparation of DNA or protein that is “substantially pure” or “isolated”+should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, unless otherwise defined, the term “antibody” includes both polyclonal and monoclonal antibodies, as well as antibody fragments having specific binding affinity for their antigen, including, but not limited to, Fv fragments, Fab fragments, Fab′ fragments, F(ab)′₂ fragments, and single chain (sFv) engineered antibody molecules. The term further includes, unless specifically excluded, chimeric and humanized antibodies, as well as human antibodies in circumstances where such antibodies can be produced.

### III. PAR1 Antagonists

Employing a PAR1 antagonist, the invention provides methods for treating or preventing cardiac dysfunctions (e.g., cardiac remodeling, hypertrophy, or heart failure) induced by or associated with cardiomyopathies, and methods for inhibiting cardiomyocyte hypertrophy, proliferation of cardiac fibroblasts or cardiac fibrosis. Various PAR1 antagonists can be employed in the practice of the therapeutic methods of the invention. These include any compounds which can inhibit one or more of the biological activities (e.g., ligand binding) and/or signaling activities of PAR1. They also include compounds which can suppress expression of PAR1 or down-regulate its cellular level. Compounds that can suppress expression of PAR1 can act at either the transcription level (i.e., preventing the transcription of mRNA that can be translated into PAR1 protein) or the translation level (i.e., preventing the translation of mRNA that has been transcribed). Suitable PAR1 antagonists encompass any PAR1 antagonist known in the art (see, e.g., G. D. Barry et al., “Agonists and Antagonists of Protease Activated Receptors,” Curr. Med. Chem. 13:243-65 (2006) (“Barry et al. (2006),” incorporated herein by this reference). Such PAR1 antagonists are disclosed in the following additional references: M. S. Bernatowicz et al., “Development of Potent Thrombin Receptor Antagonist Peptides,” J. Med. Chem. 39:48794887 (1996) (“Bernatowicz et al. (1996)”); S. M. Seiler et al. & M. S. Bernatowicz, “Peptide-Derived Protease-Activated Receptor-1 (PAR-1) Antagonists,” Curr. Med. Chem. Cardiovasc. Hematol. Agents 1:1-11 (2003) (“Seiler & Bernatowicz (2003)”); H.-C. Zhang et al., “Discovery and Optimization of a Novel Series of Thrombin Receptor (PAR-1) Antagonists: Potent, Selective Peptide Mimetics Based on Indole and Indazole Templates,” J. Med. Chem. 44:1021-1024 (2001) (“Zhang et al. (2001)”); P. Andrade-Gordon et al., “Design, Synthesis, and Biological Characterization of a Peptide-Mimetic Antagonist for a Tethered-Ligand Receptor,” Proc. Natl. Acad. Sci. USA 96:12257-12262 (1999) (“Andrade-Gordon et al. (1999)”); H.-C. Zhang et al., “High-Affinity Thrombin Receptor (PAR-1) Ligands: A New Generation of Indole-Based Peptide Mimetic Antagonists with a Basic Amine at the C-Terminus,” Bioorg. Med. Chem. Lett. 13:2199-2203 (2003); (“Zhang et al. (2003)”); B. E. Maryanoff et al., “Discovery of Potent Peptide-Mimetic Antagonists for the Human Thrombin Receptor, Protease-Activated Receptor-1 (PAR-1),” Curr. Med. Chem. Cardiovasc Hematol. Agents 1:13-36 (2003) (“Maryanoff et al. (2003)”); H.-S. Ahn et al., “Inhibition of Cellular Action of Thrombin by N3-Cyclopropyl-7-{[4-(1-Methylethyl)phenyl]methyl-7H-Pyrrolo[3,2-f]quinazoline-1,3-Diamine (SCH 79797), a Nonpeptide Thrombin Receptor Antagonist,” Biochem. Pharma. 60:1425-1434 (2000) (“Ahn et al. (2000)”); S. M. Seiler et al., “Inhibition of Thrombin and SFLLR-Peptide Stimulation of Platelet Aggregation, Phospholipase A, and Na⁺/H⁺ Exchange by a Thrombin Receptor Antagonist,” Biochem. Pharm. 49:519-528 (1995) (“Seiler et al. (1995)”); Y. Kato et al. “Inhibition of Arterial Thrombosis by a Protease-Activated Receptor 1 Antagonist, FR171113, in the Guinea Pig,” Eur. J. Pharmacol. 473: 163-169 (2003) (“Kato et al. (2003)”), Y. Kato et al., “In Vitro Antiplatelet Profile of FR171113, a Novel Non-Peptide Thrombin Receptor Antagonist,” Eur. J. Pharmacol. 384:197-202 (1999) (“Kato et al. (1999)”); W. J. Hoekstra et al., “Thrombin Receptor (PAR-1) Antagonists. Heterocycle-Based Peptidomimetics of the SFLLR Agonist Motif,” Bioorg. Med. Chem. Lett. 8:1649-1654 (1998) (“Hoekstra et al. (1998)”); R. Pakala et al., “A Peptide Analogue of Thrombin Receptor-Activating Peptide Inhibits Thrombin and Thrombin-Receptor-Activating Peptide-Induced Vascular Smooth Muscle Proliferation,” J. Cardiovasc. Pharmacol. 37: 619-629 (2001) (“Pakala et al. (2001)”); J. T. Elliott et al., “Photoactivatable Peptides Based on BMS-197525: A Potent Antagonist of the Human Thrombin Receptor (PAR-1),” Bioorg. Med. Chem. Lett. 9:279-284 (1999) (“Elliott et al. (1999)”); E. M. Ruda et al., “Identification of Small Peptide Analogues Having Agonist and Antagonist Activity at the Platelet Thrombin Receptor,” Biochem. Pharmacol. 37:2417-2426 (1988) (“Ruda et al. (1988)”); J. Doorbar & G. Winter, “Isolation of a Peptide Antagonist to the Thrombin Receptor Using Phage Display,” J. Mol. Biol. 244:361-369 (1994) (Doorbar & Winter (1994)”); S. Chackalamannil et al., “Potent Low Molecular Weight Thrombin Receptor Antagonists,” Bioorg. Med. Chem. Lett. 11: 2851-2853 (2001) (“Chackalamannil et al. (2001”); S. Chackalamannil et al., “Potent Non-Peptide Thrombin Receptor Antagonists,” Curr. Med. Chem. Cardiovasc. Hematol. Agents 1:37-45 (2003) (“Chalackamannil et al. (2003)”)); P. G. Nantermet et al., “Discovery of a Nonpeptidic Small Molecule Antagonist of the Human Platelet Thrombin Receptor,” Bioorg. Med. Chem. Lett. 12:319-23 (2002) (“Nantermet et al. (2002)”); H. G. Selnick et al., “Non-Peptidic Small Molecule Antagonists of the Human Platelet Thrombin Receptor PAR-1,” Curr. Med. Chem. Cardiovasc. Hematol. Agents 1:47-59 (2003) (“Selnick et al. (2003”); and B. P. Damiano et al., “RWJ-58259: A Selective Antagonist of Protease Activated Receptor-1,” Cardiovasc. Drug. Rev. 21:313-26 (2003) (“Damiano et al. (1993)”), all of which are incorporated herein by this reference. Additional compounds suitable for use in methods according to the present invention can be readily identified by screening candidate compounds for PAR1-antagonizing activity. PAR1 antagonists that can be employed in the present invention include antagonists which are peptides, peptidomimetics, small molecule organic compounds, pepducins, polynucleotides, or antibodies. These classes of PAR1 antagonists are discussed further below.

PAR-1 can be cleaved and activated by various proteases including MMP1, activated protein C and factor Xₐ. Inhibition of thrombin would be expected to block PAR-1 activation. In addition, blocking thrombin would inhibit fibrin deposition and activation of platelets.

In general, therefore, this method comprises administering to a subject a therapeutically effective amount of an antagonist of protease activated receptor 1 (PAR1), thereby treating or preventing cardiac dysfunction in the subject, wherein the subject:

(1) has or is at risk of developing a cardiomyopathy; or

(2) has undergone a myocardial injury.

The cardiac dysfunction can be cardiac hypertrophy, cardiac remodeling, or heart failure. In one alternative, the subject has developed or is at risk of developing an extrinsic cardiomyopathy, such as ischemic cardiomyopathy. Alternatively, the subject can have undergone a myocardial injury, such as myocardial infarction or cardiac ischemia/reperfusion. In another alternative, the subject can have developed or be at risk of developing an intrinsic cardiomyopathy, such as idiopathic dilated cardiomyopathy, viral cardiomyopathy, postpartum cardiomyopathy, or hypertrophic cardiomyopathy.

Preferably, PAR1 antagonists employed in the invention selectively inhibit PAR1 but do not affect signaling activities of other members of the protease activated receptor family (e.g., PAR2, PAR3, or PAR4). A PAR1 selective antagonist is a compound which has a high potency in inhibiting PAR1-mediated signaling activities (e.g., PAR1 binding to its tethered ligand or intracellular calcium release) but has very little or no effect on the cellular activities of the other member of the protease activated receptor family (e.g., PAR2, PAR3, or PAR4). Additionally or alternatively, PAR1-selective antagonists can be defined by their binding affinity for PAR1 such that the binding affinity of the antagonist is at least 5, 10, 25, 50, 100, 500, or 1000 fold stronger than that for any of the other PAR receptors. This difference in binding affinity is measured with respect to the binding affinity of the PAR receptor other than PAR1 for the antagonist. For example, using a radioligand binding assay, some PAR1 selective antagonists preferably bind to PAR1 with an IC₅₀ that is less than 1 μM, more preferably less than 500 nM, still more preferably less than 250 nM, still more preferably less than 100 nM, and most preferably less than 50 nM. On the other hand, these PAR1 selective antagonists preferably bind to all of the PAR2, PAR3, and PAR4 receptors with an IC₅₀ that is at least 5 μM or higher; in some cases, they may not bind to one or more of the PAR2, PAR3, and PAR4 receptors. Radioligand binding assays for determining affinity of a compound for PARs are described in the art, e.g., Bernatowicz et al. (1996), supra; Seiler & Bernatowicz, (2003), Zhang et al. (2001), supra; Andrade-Gordon et al. (1999), supra; Zhang et al. (2003), supra; Maryanoff et al. (2003), supra; and Ahn et al. (2000), supra. Compounds that selectively bind to PAR1 can also be examined with a Ca²⁺ release assay as reported in, e.g., Zhang et al. (2001), supra; Andrade-Gordon et al. (1999); supra; Ahn et al. (2000), supra; Seiler et al. (1995), supra; Kato et al. (2003), supra; and Kato et al. (1999) supra.

Some of the PAR1 antagonists used in the invention do not inhibit thrombin activity, e.g., cleavage of PAR1 by thrombin or other proteases. Rather, they modulate PAR1 signaling by inhibiting binding of the tethered ligand to the cleaved PAR1 molecule or interfering with downstream signaling activities mediated by activated PAR1, e.g., calcium mobilization.

Particular PAR1 antagonists suitable for use in methods according to the present invention include a series of modified peptides in which the amino-terminus is substituted with a number of groups as described below and the carboxyl-terminus is derivatized with an amino group to form an amide. These peptides are of the sequence X₁-F(f)F(Gn)LR-NH₂, where X₁ is as defined below, F(f) is a substituted phenylalanine residue with a fluoro group at the para position of the phenyl moiety of the phenylalanine, as shown below in Formula (I), in which X is F, and F(Gn) is a substituted phenylalanine residue with a guanidino group at the para position of the phenyl moiety of the phenylalanine, as shown below in Formula (I), in which X is NH—CNH—NH₂ or guanidino (Gn). In these antagonists, X₁ can be selected from the group consisting of (2-thiophene)acetyl, N-acetyl-2-aminobenzoyl, 2-oxo-(2-thiophene)acetyl, (3-thiophene)acetyl, phenylacetyl, (2-thiophene)sulfonyl, (3-fluorophenyl)acetyl, (4-fluorophenyl)acetyl, 3-pyridylacetyl, (2-fluorophenyl)acetyl, (3-indole)acetyl, cyclopentylacetyl, 2-oxo(3-indole)acetyl, 3-indoloyl, (3-chlorophenyl)acetyl, N-acetyl-4-aminobutyryl, 2-thiopheneoyl, 3-thiopheneoyl, 3-furanoyl, 2-indoloyl, 4-chlorobenzoyl, trans-cinnamoyl, 3-phenyl-2-propynoyl, p-fluoro-trans-cinnamoyl, p-chloro-trans-cinnamoyl, p-methyl-trans-cinnamoyl, p-methoxy-trans-cinnamoyl, 4-biphenyloyl, m-chloro-trans-cinnamoyl, 3-phenylpropionyl, phenoxyacetyl, 1-napthtylacetyl, 3-(2-thiophene)-trans-acryloyl, (±)-trans-3-phenylcyclopropanoyl, 3-coumarinyl, 4-phenylbutyryl, p-amino-trans-cinnamoyl, 2-naphthylacetyl, p-hydroxy-trans-cinnamoyl, (thiophenoxy)acetyl, trans-2-trans-4-hexadienoyl, trans-2-octenoyl, α-fluorocinnamoyl, α-methylcinnamoyl, and α-phenylcinnamoyl. A particularly preferred substituent X₁ is trans-cinnamoyl, resulting in N-trans-cinnamoyl-p-fluoroPhe-p-guanidinoPhe-Leu-Arg-NH₂ (BMS-197525). Other analogues of these peptides in which X₁ is trans-cinnamoyl and in which the NH₂ group of the trans-cinnamoyl moiety is further substituted are also effective antagonists; these substituents are shown below. These antagonists are described in Bernatowicz et al. (1996), supra.

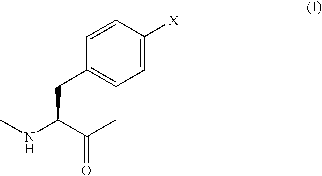

Another class of antagonists has the structure trans-cinnamoyl-F(f)-F(Gn)-L-X₂, in which X₂ is selected from the group consisting of Orn-NH₂, Orn(acetyl)-NH₂, Arg-Orn-NH₂, Arg-Orn(N⁵-acetyl)-NH₂, Arg-Arg-NH₂, and Arg-Orn(N⁵-propionyl)-NH₂. In these antagonists, F(f) and F(Gn) are as defined above with reference to Formula (I). These antagonists are also described in Bernatowicz et al. (1996), supra, and in Barry et al. (2006), supra.

Other antagonists are (N-[3-methyl-1-S[[2-S-[(methyl)aminocarbonyl]-1-pyrrolidinyl]carbonyl]butyl-D-alanine (SC40476), and N-[3-methyl-S-(1-pyrrolidinylcarbonyl)butyl-D-alanine ethyl ester hydrochloride (SC42619), both disclosed in Ruda et al. (1988), supra.

Still other PAR1 antagonists are photoactivatable analogues of BMS-197525, disclosed in Elliott et al. (1998), supra.

Another PAR1 antagonist is a non-peptidic PAR1 antagonist, FR171113, which has the structure shown below in Formula (II). This is described in Kato et al. (1999), supra, and in Barry et al. (2006), supra.

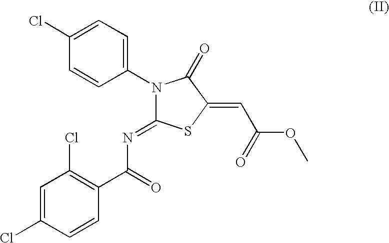

Still other PAR1 antagonists are trisubstituted ureas described in Barrow et al. (2001), supra, and Barry et al. (2006), supra. In general, these trisubstituted ureas are of Formula (III) or of Formula (IV) below. In the case of Formula (III), n can be 0, 1, or 2; in most cases, n is preferably 1. In Formula (III), R can be t-butyl-, NH₂CMe₂-, 2,4-difluorophenyl-, 3-pyridyl, 4-(SO₂NH₂)phenyl- and 4-benzimidazole-. Preferably, when n is 1, R is 4-benzimidazole-. In another preferred alternative, n is 2 and R is 4-benzimidazole-. In other alternatives, based on Formula (III), the substituent on the nitrogen atom in the central urea moiety, which is 2-propyl in Formula (III), can be altered. Particularly preferred substituents are n-propyl, cyclobutyl, i-propyl, (±)2-pentyl, (S)sec-butyl, or (R)sec-butyl. For compounds of Formula (IV), X can be CH₂, S, or SO; R can be hydrogen, ethyl, or methyl; various alternatives are possible for the stereochemistry at the 2, 2′, and 5 positions (such as S,S; S,R; R,S; R,R; S,S-rac; S,S,S; and S,S,); and n can be 1 or 2. For a particularly preferred compound of Formula (IV), X is CH₂, R is hydrogen, the stereochemistry at the 2 and 2′ positions is S,S, and n is 1.

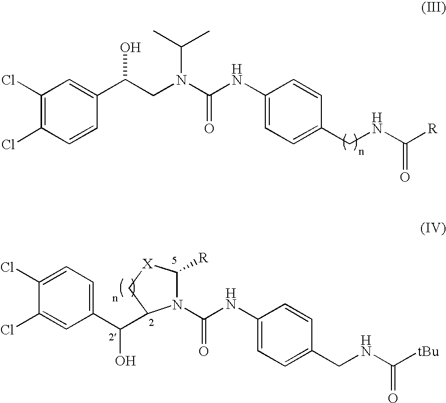

Other PAR1 antagonist compounds include the isoxazole compound shown below as Formula (V) and derivatives thereof. These derivatives include compounds of Formula (VI) in which R is 3-methyl, 4-methyl, 3-methoxy, 4-methoxy, or 3,5-difluoro. These compounds are shown in Nantermet et al. (2002), supra, and in Barry et al. (2006), supra.

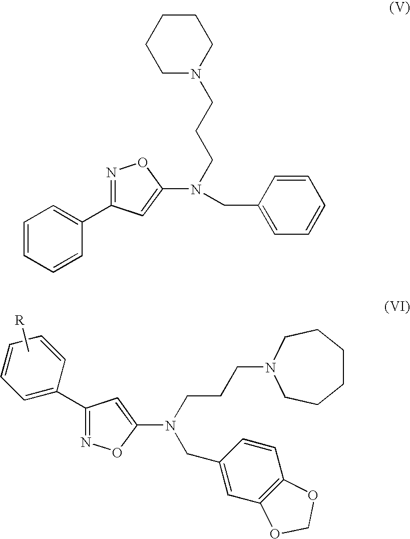

Still other PAR1 antagonist compounds include a derivative of a 1,3-dihydrobenzoimidazolamine analogue, ER-1296614-06, shown below as Formula (VII). This compound is described in Barry et al. (2006), supra.

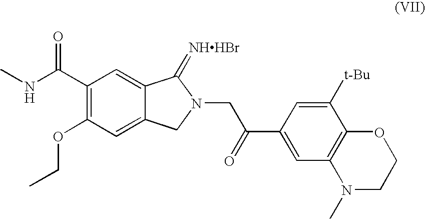

Still other PAR1 antagonist compounds include pyrroloquinazolines, including SCH 79797, discussed below, and SCH 203099, shown below as Formula (VIII). These are disclosed in Ahn et al. (1999), supra, and Barry et al. (2006), supra.

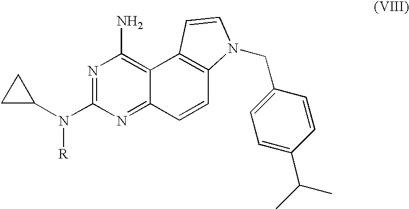

Still other PAR1 antagonist compounds include benzimidazole derivatives, including a compound of Formula (IX) in which R is benzyl and R₁ is t-butyl. In other alternatives of compounds of Formula (IX), R can be hydrogen, methyl, n-butyl, or 4-methylbenzyl and R₁ can be hydrogen or t-butyl. These are disclosed in Chackalamannil et al. (2001), supra, and Barry et al. (1996), supra.

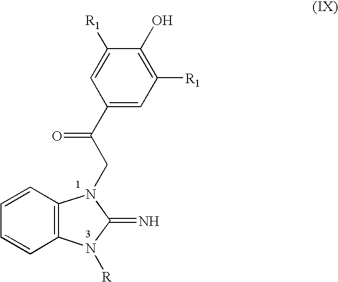

Still other PAR1 antagonist compounds are derivatives of the natural product himbacine, which has activity at muscarinic receptors and was originally studied as part of an Alzheimer's disease program. A particularly preferred himbacine derivative is SCH-530348, shown below in Formula (X). SCH-530348 is described in Barry et al. (2006), supra.

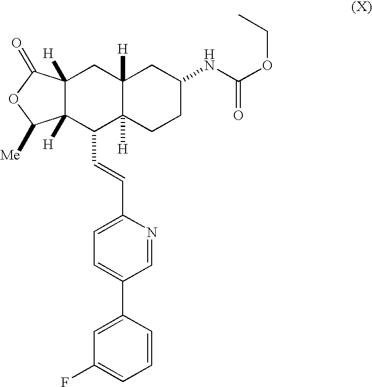

Other analogues and derivatives of himbacine with PAR1 antagonist activity are known in the art and are described in U.S. Patent Application Publication No. 2004/0152736 by Chackalamannil et al.; U.S. Patent Application Publication No. 2004/1076418 by Thiruvengadam et al.; U.S. Patent Application Publication No. 2004/0192753 by Chackalamannil et al.; U.S. Patent Application Publication No. 2003/0216437 by Chackalamannil et al.; U.S. Patent Application Publication No. 2003/0203927 by Chackalamannil et al.; U.S. Patent Application Publication No. 2008/0026050 by Gupta et al.; U.S. Patent Application Publication No. 2007/0238674 by Veltri et al.; U.S. Pat. No. 6,645,987 to Chackalamannil et al.; U.S. Pat. No. 6,063,847 to Chackalamannil et al.; PCT Patent Application Publication No. WO 2007/075964 by Veltri et al; and PCT Patent Application Publication No. WO 2007/117621 by Veltri et al., all of which are incorporated herein by this reference.

Iminopyrrolidine derivatives with PAR1 antagonist activity have been described in U.S. Pat. No. 7,244,730 to Suzuki et al. and EPO Patent Application Publication No. EP 1813282 by Hirano et al., both of which are incorporated herein by this reference.

Most generally, these iminopyrrolidine derivatives have the structure of Formula (XI), below, in which; ring A indicates a pyrrolidine ring; ring B indicates a benzene ring or a pyridine ring; R¹⁰¹, R¹⁰², and R¹⁰³ each independently indicate, identically or differently, a hydrogen atom, a halogen atom, a C₁-C₆ alkyl group, or a C₁-C₆ alkoxy-substituted C₁-C₆ alkyl group; R⁵ indicates a hydrogen atom; R⁶ indicates a hydrogen atom, a C₁-C₆ alkyl group, or a C₁-C₆ alkyloxy-carbonyl group; Y¹ indicates a single valence bond or a —CH₂— group; Y² indicates a single valence bond or a —CO— group; Ar¹ indicates a hydrogen atom or a group represented by Formula (XII), below. In Formula (XII), R¹⁰, R¹¹, R¹², R¹³, and R¹⁴ independently indicate, identically, or differently, a hydrogen atom, a C₁-C₆ alkyl group, or a C₁-C₆ alkoxy group, a morpholinyl group, a piperazinyl group which can be substituted or unsubstituted, or a piperidinyl group which can be substituted or unsubstituted, and R¹¹, R¹², and R¹³ can form a 5-8 membered heterocycle, or a pharmaceutically acceptable salt or hydrate of a compound of Formula (XI).

More preferably, the 2-iminopyrrolidine compound is a compound of Formula (XIII), wherein R¹ and R² independently indicate, identically or differently, a hydrogen atom, a methoxy group, or an ethoxy group; X¹ indicates a hydrogen atom or a halogen atom; Ar² indicates a substituted or unsubstituted methyl group, a substituted or unsubstituted ethyl group, a substituted or unsubstituted methoxy group, a substituted or unsubstituted ethoxy group, a substituted or unsubstituted t-butyl group, a substituted or unsubstituted morpholino group, or a substituted or unsubstituted phenyl group where these groups can be substituted with one or more substituents selected substituents represented by Formula (XIV), wherein, in Formula (XIV), W indicates —CH— or a nitrogen atom; A¹ indicates —CH₂— or a single valence bond; R³ indicates a hydrogen atom or —OR^{5a}; X² indicates —CH₂—, an oxygen atom, a single valence bond, or a carbonyl group; Y indicates a single valence bond or a C₁-C₄ alkylene group; R⁴ indicates a hydrogen atom, —OR^{6a}, a cyano group or —COOR⁷; R^{5a}, R^{6a}, and R⁷ independently indicate, identically or differently, a hydrogen atom or a C₁-C₄ alkyl group; or a pharmaceutically acceptable salt or hydrate of a compound of Formula (XIII).

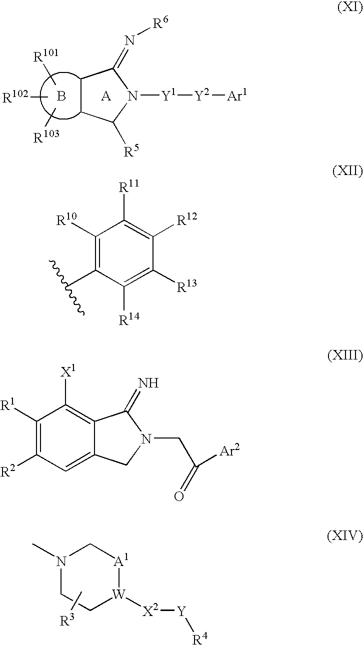

Preferred 2-iminopyrrolidine antagonists are compounds of Formulas (XV) through (XXI), shown below. A particularly preferred 2-iminopyrrolidine antagonist is a compound of Formula (XV), designated as E5555.

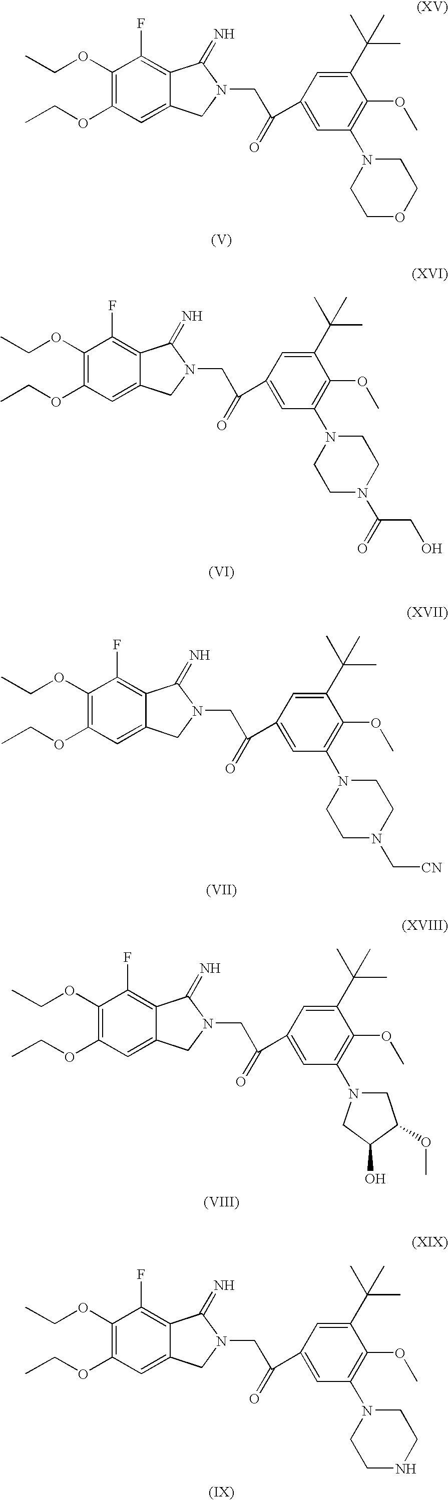

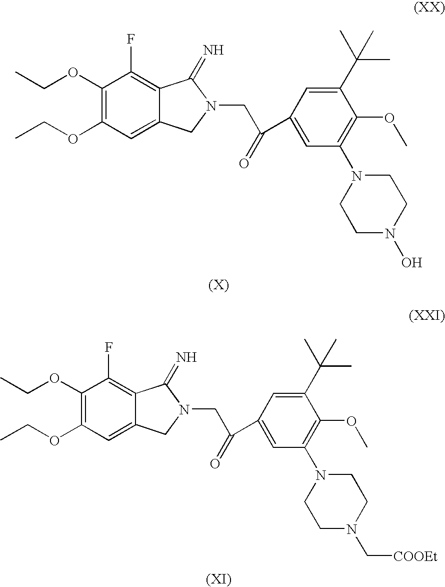

Additional 2-iminopyrrolidine PAR1 antagonists and PAR1 antagonists that are analogues or derivatives of 2-iminopyrrolidine compounds are disclosed in U.S. Pat. No. 7,244,730 to Suzuki et al. These compounds include, but are not limited to: 2-[2-(3,5-di-t-butyl-4-hydroxy-phenyl)-2-oxo-ethyl]-6-ethoxy-3-imino-2,-3-dihydro-1H-isoindole-5-carboxylic acid methylamide trifluoroacetate; 2-[2-(3,5-di-t-butyl-4-hydroxy-phenyl)-2-oxo-ethyl]-6-dimethylamino-3-imino-2,3-dihydro-1H-isoindole-5-carboxylic acid methylamide hydrobromide; 1-(3,5-di-t-butyl-4-hydroxy-phenyl)-2-(7-fluoro-1-imino-5,6-dimethoxy 1,3-dihydro-isoindol-2-yl)-ethanone hydrobromide; 1-(8-t-butyl-4-methyl-3,4-dihydro-2H-benzo[1,4]oxazin-6-yl)-2-(5,6-diethoxy-7-fluoro-1-imino-1,3-dihydro-isoindol-2-yl)-ethanone hydrobromide; 2-[2-(3-t-butyl-4,5-dimethoxy-phenyl)-2-oxo-ethyl]-6-ethoxy-3-imino-2,-3-dihydro-1H-isoindole-5-carboxylic acid methylamide hydrobromide; 1-(3,5-di-t-butyl-4-hydroxy-phenyl)-2-(5-ethoxy-7-fluoro-1-imino-6-methoxy-1,3-dihydro-isoindol-2-yl)-ethanone hydrobromide; 2-[2-(3-t-butyl-4-hydroxy-5-isopropoxy-phenyl)-2-oxo-ethyl]-6-ethoxy-3-imino-2,3-dihydro-1H-isoindole-5-carboxylic acid methylamide hydrobromide; 6-[2-(8-t-butyl-4-methyl-3,4-dihydro-2H-benzo[1,4]oxazin-6-yl)-2-oxo-ethyl]-3-ethoxy-7-imino-6,7-dihydro-5H-pyrrolo[3,4-b]pyridine-2-carboxylic acid methylamide hydrochloride; 2-[2-(7-t-butyl-3-methyl-3H-benzimidazol-5-yl)-2-oxo-ethyl]-6-ethoxy-3-imino-2,3-dihydro-1H-isoindole-5-carboxylic acid methylamide hydrobromide; 2-[2-(3-t-butyl-5-dimethylamino-4-hydroxy-phenyl)-2-oxo-ethyl]-6-ethoxy-3-imino-2,3-dihydro-1H-isoindole-5-carboxylic acid methylamide hydrobromide; 5-{2-tert-butyl-4-[2-(2-cyclopropyl-7-imino-5,7-dihydro-pyrrolo[3,4-b]pyridin-6-yl)-acetyl]-6-(pyrrolidin-1-yl)-phenoxy}-pentanoic acid trifluoroacetate; 5-{2-t-butyl-4-[2-(7-fluoro-1-imino-5,6-dimethoxy-1,3-dihydro-isoindol-2-yl)-acetyl]-6-(pyrrolidin-1-yl)-phenoxy}-pentanoic acid trifluoroacetate; 2-[2-(3-t-butyl-5-dimethylamino-4-methoxy-phenyl)-2-oxo-ethyl]-6-ethoxy-3-imino-2,3-dihydro-1H-isoindole-5-carboxylic acid methylamide hydrobromide; 2-(1-(3-t-butyl-5-[2-(7-fluoro-1-imino-5,6-dimethoxy-1,3-dihydro-isoindol-2-yl)-acetyl]-2-methoxy-phenyl)-pyrrolidin-3-yloxy)-butyric acid trifluoroacetate; 2-(1-{3-t-butyl-5-[2-(7-fluoro-1-imino-5,6-dimethoxy-1,3-dihydro-isoindol-2-yl)-acetyl]-2-methoxy-phenyl)-pyrrolidin-3-yloxy)-butyric acid trifluoroacetate; 1-(3-t-butyl-5-dimethylamino-4-methoxy-phenyl)-2-(7-fluoro-1-imino-5,6-dimethoxy-1,3-dihydro-isoindol-2-yl)-ethanone hydrobromide; 2-[2-(3-t-butyl-5-dimethylamino-4-methoxy-phenyl)-2-oxo-ethyl]-6-dimethylamino-3-imino-2,3-dihydro-1H-isoindole-5-carboxylic acid methylamide hydrobromide; 6-[2-(3-t-butyl-5-dimethylamino-4-methoxy-phenyl)-2-oxo-ethyl]-3-ethoxy-7-imino-6,7-dihydro-5H-pyrrolo[3,4-b]pyridine-2-carboxylic acid methylamide hydrochloride; 2-[2-(3-t-butyl-5-ethoxy-4-methoxy-phenyl)-2-oxo-ethyl]-6-dimethylamino-3-imino-2,3-dihydro-1H-isoindole-5-carboxylic acid methylamide hydrobromide; 2-[2-(3-t-butyl-5-ethoxy-4-methoxy-phenyl)-2-oxo-ethyl]-6-ethoxy-3-imino-2,3-dihydro-1H-isoindole-5-carboxylic acid methylamide hydrobromide; 1-(3-t-butyl-5-ethoxy-4-methoxy-phenyl)-2-(5,6-diethoxy-7-fluoro-1-imino-1,3-dihydro-isoindol-2-yl)-ethanone hydrobromide; 6-[2-(3-t-butyl-5-ethoxy-4-methoxy-phenyl)-2-oxo-ethyl]-3-ethoxy-7-imino-6,7-dihydro-5H-pyrrolo[3,4-b]pyridine-2-carboxylic acid methylamide trifluoroacetate; {3-t-butyl-5-[2-(7-fluoro-1-imino-5,6-dimethoxy-1,3-dihydro-isoindol-2-yl)-acetyl]-2-methoxy-phenoxy}-acetonitrile hydrobromide; 4-(3-t-butyl-5-[2-(7-fluoro-1-imino-5,6-dimethoxy-1,3-dihydro-isoindol-2-yl)-acetyl]-2-methoxy-phenoxy)-butyronitrile hydrobromide; 2-[2-(3-t-butyl-5-cyanomethoxy-4-methoxy-phenyl)-2-oxo-ethyl]-6-dimethylamino-3-imino-2,3-dihydro-1H-isoindole-5-carboxylic acid methylamide hydrobromide; 2-{2-[3-t-butyl-5-(3-cyano-propoxy)-4-methoxy-phenyl]-2-oxo-ethyl)-6-dimethylamino-3-imino-2,3-dihydro-1H-isoindole-5-carboxylic acid methylamide hydrobromide; 2-[2-(8-t-butyl-4-cyanomethyl-3,4-dihydro-2H-benzo[1,4]oxazin-6-yl)-2-oxo-ethyl]-6-ethoxy-3-imino-2,3-dihydro-1H-isoindole-5-carboxylic acid methylamide trifluoroacetate; 6-[2-(8-t-butyl-4-cyanomethyl-3,4-dihydro-2H-benzo[1,4]oxazin-6-yl)-2-oxo-ethyl]-3-ethoxy-7-imino-6,7-dihydro-5H-pyrrolo[3,4-b]pyridine-2-carboxylic acid methylamide trifluoroacetate; (8-t-butyl-6-[2-(7-fluoro-1-imino-5,6-dimethoxy-1,3-dihydro-isoindol-2-yl)-acetyl]-2,3-dihydro-benzo[1,4]oxazin-4-yl)-acetonitrile trifluoroacetate; {8-t-butyl-6-[2-(5,6-diethoxy-7-fluoro-1-imino-1,3-dihydro-isoindol-2-yl)-acetyl]-2,3-dihydro-benzo[1,4]oxazin-4-yl}-acetonitrile trifluoroacetate; (8-t-butyl-6-[2-(2-cyclopropyl-7-imino-5,7-dihydro-pyrrolo[3,4-b]pyridin-6-yl)-acetyl]-2,3-dihydro-benzo[1,4]oxazin-4-yl)-acetonitrile trifluoroacetate; 2-[2-(8-t-butyl-4-cyanomethyl-3,4-dihydro-2H-benzo[1,4]oxazin-6-yl)-2-oxo-ethyl]-6-dimethylamino-3-imino-2,3-dihydro-1H-isoindole-5-carboxylic acid methylamide trifluoroacetate; 2-[2-(3-t-butyl-5-cyanomethoxy-4-methoxy-phenyl)-2-oxo-ethyl]-6-ethoxy-3-imino-2,3-dihydro-1H-isoindole-5-carboxylic acid methylamide hydrobromide; 2-(2-[3-t-butyl-5-(3-cyano-propoxy)-4-methoxy-phenyl]-2-oxo-ethyl}-6-ethoxy-3-imino-2,3-dihydro-1H-isoindole-5-carboxylic acid methylamide hydrobromide; 1-(3-t-butyl-5-ethoxy-4-methoxy-phenyl)-2-(7-fluoro-1-imino-5,6-dimethoxy-1,3-dihydro-isoindol-2-yl)-ethanone hydrobromide; 2-[2-(3-t-butyl-4-methoxy-5-morpholino-phenyl)-2-oxo-ethyl]-6-ethoxy-3-imino-2,3-dihydro-1H-isoindole-5-carboxylic acid methylamide hydrobromide; 1-(3-t-butyl-4-methoxy-5-morpholino-phenyl)-2-(7-fluoro-1-imino-5,6-di-methoxy-1,3-dihydro-isoindol-2-yl)-ethanone hydrobromide; 1-(3-t-butyl-4-methoxy-5-morpholino-phenyl)-2-(2-cyclopropyl-7-imino-5-,7-dihydro-pyrrolo[3,4-b]pyridin-6-yl)-ethanone hydrobromide; (3-t-butyl-5-[2-(5,6-diethoxy-7-fluoro-1-imino-1,3-dihydro-isoindol-2-yl)-acetyl]-2-methoxy-phenoxy)-acetonitrile hydrobromide; 4-{3-t-butyl-5-[2-(5,6-diethoxy-7-fluoro-1-imino-1,3-dihydro-isoindol-2-yl)-acetyl]-2-methoxy-phenoxy)-butyronitrile hydrobromide; 4-{3-t-butyl-5-[2-(2-cyclopropyl-7-imino-5,7-dihydro-pyrrolo[3,4-b]pyridin-6-yl)-acetyl]-2-methoxy-phenoxy}-butyronitrile hydrobromide; 1-(3-t-butyl-5-dimethylamino-4-methoxy-phenyl)-2-(5,6-diethoxy-7-fluor-o-1-imino-1,3-dihydro-isoindol-2-yl)-ethanone hydrobromide; 1-(3-t-butyl-4-methoxy-5-morpholino-phenyl)-2-(5,6-diethoxy-7-fluoro-1-imino-1,3-dihydro-isoindol-2-yl)-ethanone hydrobromide; 6-[2-(3-t-butyl-4-methoxy-5-morpholino-phenyl)-2-oxo-ethyl]-3-ethoxy-7-imino-6,7-dihydro-5H-pyrrolo[3,4-b]pyridine-2-carboxylic acid methylamide hydrobromide; 2-t-butyl-4-[2-(5-ethoxy-1-imino-6-methylcarbamoyl-1,3-dihydro-isoindol-2-yl)-acetyl]-phenyl methanesulfonate hydrobromide; 2-t-butyl-4-[2-(5-dimethylamino-1-imino-6-methylcarbamoyl-1,3-dihydroisoindol-2-yl)-acetyl]-phenyl methanesulfonate hydrobromide; 2-t-butyl-4-[2-(3-ethoxy-7-imino-2-methylcarbamoyl-5,7-dihydro-pyrrolo-[3,4-b]pyridin-6-yl)-acetyl]-phenyl methanesulfonate hydrobromide; 2-[2-(3-t-butyl-4-cyanomethoxy-5-dimethylamino-phenyl)-2-oxo-ethyl]-6-dimethylamino-3-imino-2,3-dihydro-1H-isoindole-5-carboxylic acid methylamide hydrobromide; 2-[2-(3-t-butyl-4-methoxy-5-(pyrrolidin-1-yl)-phenyl)-2-oxo-ethyl]-6-ethoxy-3-imino-2,3-dihydro-1H-isoindole-5-carboxylic acid methylamide hydrobromide; 1-(3-t-butyl-4-methoxy-5-(pyrrolidin-1-yl)-phenyl)-2-(5,6-diethoxy-7-fluoro-1-imino-1,3-dihydro-isoindol-2-yl)-ethanone hydrobromide; 2-[2-(3-t-butyl-4-methoxy-5-(pyrrolidin-1-yl)-phenyl)-2-oxo-ethyl]-6-dimethylamino-3-imino-2,3-dihydro-1H-isoindole-5-carboxylic acid methylamide hydrobromide; 1-(3-t-butyl-4-methoxy-5-(pyrrolidin-1-yl)-phenyl)-2-(2-cyclopropyl-7-imino-5,7-dihydro-pyrrolo[3,4-b]pyridin-6-yl)-ethanone hydrobromide; 1-(3-t-butyl-4-methoxy-5-(pyrrolidin-1-yl)-phenyl)-2-(7-fluoro-1-imino-5,6-dimethoxy-1,3-dihydro-isoindol-2-yl)-ethanone hydrobromide; 2-[2-(3-t-butyl-5-isopropoxy-4-methoxy-phenyl)-2-oxo-ethyl]-6-ethoxy-3-imino-2,3-dihydro-1H-isoindole-5-carboxylic acid methylamide hydrobromide; 1-(3-t-butyl-5-isopropoxy-4-methoxy-phenyl)-2-(5,6-diethoxy-7-fluoro-1-imino-1,3-dihydro-isoindol-2-yl)-ethanone hydrobromide; 1-(3-t-butyl-5-isopropoxy-4-methoxy-phenyl)-2-(7-fluoro-1-imino-5,6-di-methoxy-1,3-dihydro-isoindol-2-yl)-ethanone hydrobromide; ethyl 2-{8-t-butyl-6-[2-(5-ethoxy-1-imino-6-methylcarbamoyl-1,3-dihydro-isoindol-2-yl)-acetyl}-2,3-dihydro-benzo[1,4]oxazin-4-yl)-propionate hydrochloride; ethyl 2-{8-t-butyl-6-[2-(3-ethoxy-7-imino-2-methylcarbamoyl-5,7-dihydro-pyrrolo[3,4-b]pyridin-6-yl)-acetyl]-2,3-dihydro-benzo[1,4]oxazin-4-yl}-propionate hydrochloride; 2-[2-(3-dimethylamino-5-isopropyl-4-methoxy-phenyl)-2-oxo-ethyl]-6-ethoxy-3-imino-2,3-dihydro-1H-isoindole-5-carboxylic acid methylamide hydrobromide; 2-(5,6-diethoxy-7-fluoro-1-imino-1,3-dihydro-isoindol-2-yl)-1-(3-dimethyl-amino-5-isopropyl-4-methoxy-phenyl)-ethanone hydrobromide; 2-[2-(3-t-butyl-4-methoxy-5-methylamino-phenyl)-2-oxo-ethyl]-6-ethoxy-3-imino-2,3-dihydro-1H-isoindole-5-carboxylic acid methylamide hydrobromide; 6-[2-(3-t-butyl-5-isopropoxy-4-methoxy-phenyl)-2-oxo-ethyl]-3-ethoxy-7-imino-6,7-dihydro-5H-pyrrolo[3,4-b]pyridine-2-carboxylic acid methylamide trifluoroacetate, 2-[2-(3-t-butyl-5-isopropoxy-4-methoxy-phenyl)-2-oxo-ethyl]-6-dimethyl-amino-3-imino-2,3-dihydro-1H-isoindole-5-carboxylic acid methylamide trifluoroacetate; 2-{2-[3-t-butyl-5-(4-cyano-piperidin-1-yl)-4-methoxy-phenyl]-2-oxo-ethyl}-6-ethoxy-3-imino-2,3-dihydro-1H-isoindole-5-carboxylic acid methylamide hydrobromide; 1-(3-t-butyl-4-hydroxy-phenyl)-2-(5,6-diethoxy-7-fluoro-1-imino-1,3-dihydro-isoindol-2-yl)-ethanone hydrobromide; 2-{8-t-butyl-6-[2-(5-ethoxy-1-imino-6-methylcarbamoyl-1,3-dihydro-isoindol-2-yl)-acetyl}-2,3-dihydro-benzo[1,4]oxazin-4-yl}-2-methyl-propanoic acid hydrochloride; 2-{8-t-butyl-6-[2-(3-ethoxy-7-imino-2-methylcarbamoyl-5,7-dihydro-pyrrolo[3,4-b]pyridin-6-yl)-acetyl]-2,3-dihydro-benzo[1,4]oxazin-4-yl}-2-methyl-propanoic acid hydrochloride; 2-t-butyl-6-dimethylamino-4-[2-(5-ethoxy-1-imino-6-methylcarbamoyl-1,3-dihydro-isoindol-2-yl)-acetyl]-phenyl acetate hydrobromide; 2-{2-[3-t-butyl-4-methoxy-5-(2-oxo-oxazolidin-3-yl)-phenyl]-2-oxo-ethyl}-6-ethoxy-3-imino-2,3-dihydro-1H-isoindole-5-carboxylic acid methylamide hydrobromide; 2-t-butyl-4-[2-(5,6-diethoxy-7-fluoro-1-imino-1,3-dihydro-isoindol-2-yl)-acetyl]-phenyl acetate hydrobromide; 2-t-butyl-4-[2-(5-ethoxy-1-imino-6-methylcarbamoyl-1,3-dihydro-isoindol-2-yl)-acetyl]-phenyl acetate hydrobromide; 1-{3-t-butyl-5-[2-(5,6-diethoxy-7-fluoro-1-imino-1,3-dihydro-isoindol-2-yl)-acetyl]-2-methoxy-phenyl}-piperidin-4-one hydrobromide; 1-(3-t-butyl-5-dimethylamino-4-methoxy-phenyl)-2-(5-ethoxy-7-fluoro-1-imino-6-methoxy-1,3-dihydro-isoindol-2-yl)-ethanone hydrobromide; 2-{2-[3-t-butyl-5-(ethyl-methyl-amino)-4-methoxy-phenyl]-2-oxo-ethyl}-6-ethoxy-3-imino-2,3-dihydro-1H-isoindole-5-carboxylic acid methylamide hydrobromide; 6-{2-[3-t-butyl-5-(ethyl-methyl-amino)-4-methoxy-phenyl]-2-oxo-ethyl}-3-ethoxy-7-imino-6,7-dihydro-5H-pyrrolo[3,4-b]pyridine-2-carboxylic acid methylamide trifluoroacetate; 2-t-butyl-4-[2-(5,6-diethoxy-7-fluoro-1-imino-1,3-dihydro-isoindol-2-yl)-acetyl]-6-dimethylamino-phenyl methanesulfonate hydrobromide; 1-[3-t-butyl-5-(4-hydroxy-piperidin-1-yl)-4-methoxy-phenyl]-2-(5,6-diethoxy-7-fluoro-1-imino-1,3-dihydro-isoindol-2-yl)-ethanone hydrobromide; 6-{2-[3-t-butyl-5-(4-hydroxy-piperidin-1-yl)-4-methoxy-phenyl]-2-oxo-ethyl}-3-ethoxy-7-imino-6,7-dihydro-5H-pyrrolo[3,4-b]pyridine-2-carboxylic acid methylamide trifluoroacetate; 1-[3-t-butyl-5-(4-hydroxy-piperidin-1-yl)-4-methoxy-phenyl]-2-(7-fluoro-1-imino-5,6-dimethoxy-1,3-dihydro-isoindol-2-yl)-ethanone hydrobromide; 1-(3-t-butyl-5-dimethylamino-4-hydroxy-phenyl)-2-(5,6-diethoxy-7-fluoro-1-imino-1,3-dihydro-isoindol-2-yl)-ethanone hydrobromide; 6-[2-(3-t-butyl-5-dimethylamino-4-ethoxy-phenyl]-2-oxo-ethyl]-3-ethoxy-7-imino-6,7-dihydro-5H-pyrrolo[3,4-b]pyridine-2-carboxylic acid methylamide trifluoroacetate; 2-[2-(3-t-butyl-5-dimethylamino-4-methoxy-phenyl)-2-oxo-ethyl]-6-dimethylamino-3-imino-2,3-dihydro-1H-isoindole-5-carboxylic acid methylamide hydrobromide; 1-(3-t-butyl-4-methoxy-5-methylamino-phenyl)-2-(5,6-diethoxy-7-fluoro-1-imino-1,3-dihydro-isoindol-2-yl)-ethanone hydrobromide; 1-[3-t-butyl-5-(4-hydroxy-piperidin-1-yl)-4-methoxy-phenyl]-2-(5-ethoxy-7-fluoro-1-imino-6-methoxy-1,3-dihydro-isoindol-2-yl hydrobromide; 2-(5,6-diethoxy-7-fluoro-1-imino-1,3-dihydro-isoindol-2-yl)-1-[3-dimethyl-amino-5-(1-fluoro-1-methyl-ethyl)-4-methoxy-phenyl]-ethanone hydrobromide; 1-[3-t-butyl-5-(3-hydroxy-pyrrolidin-1-yl)-4-methoxy-phenyl]-2-(5,6-di-ethoxy-7-fluoro-1-imino-1,3-dihydro-isoindol-2-yl)-ethanone trifluoroacetate; 2-[2-(3-t-butyl-5-ethylamino-4-methoxy-phenyl)-2-oxo-ethyl]-6-ethoxy-3-imino-2,3-dihydro-1H-isoindole-5-carboxylic acid methylamide hydrobromide; 2-[2-(3-t-butyl-5-ethylamino-4-methoxy-phenyl)-2-oxo-ethyl]-6-ethoxy-3-imino-2,3-dihydro-1H-isoindole-5-carboxylic acid methylamide hydrobromide; 1-(3-t-butyl-5-ethoxy-4-hydroxy-phenyl)-2-(5,6-diethoxy-7-fluoro-1-imino-1,3-dihydro-isoindol-2-yl)-ethanone hydrobromide; 2-[2-(3-t-butyl-5-ethoxy-4-hydroxy-phenyl)-2-oxo-ethyl]-6-ethoxy-3-imino-2,3-dihydro-1H-isoindole-5-carboxylic acid methylamide hydrobromide; 2-t-butyl-4-[2-(5,6-diethoxy-7-fluoro-1-imino-1,3-dihydro-isoindol-2-y-l)-acetyl]-6-ethoxy-phenyl ethyl-carbamate hydrobromide; 2-t-butyl-6-ethoxy-4-[2-(5-ethoxy-1-imino-6-methylcarbamoyl-1,3-dihydro-isoindol-2-yl)-acetyl]-phenyl ethyl-carbamate hydrobromide; 2-t-butyl-6-[(3-cyano-propoxy)-4-[2-(5,6-diethoxy-7-fluoro-1-imino-1,3-dihydro-isoindol-2-yl)-acetyl]-phenyl methanesulfonate hydrobromide; 1-(3-t-butyl-4-methoxy-5-piperazin-1-yl-phenyl)-2-(5,6-diethoxy-7-fluoro-1-imino-1,3-dihydro-isoindol-2-yl)-ethanone dihydrochloride; 2-(2-{3-t-butyl-4-methoxy-5-[(2-methoxyethyl)-methylamino]-phenyl}-2-oxo-ethyl)-6-dimethylamino-3-imino-2,3-dihydro-1H-isoindole-5-carboxylic acid methylamide hydrobromide; 1-[3-t-butyl-5-(2-hydroxyethylamino)-4-methoxy-phenyl]-2-(5,6-diethoxy-7-fluoro-1-imino-1,3-dihydro-isoindol-2-yl)-ethanone hydrochloride; 1-{3-t-butyl-5-[(2-hydroxyethyl)-methylamino]-4-methoxy-phenyl}-2-(5,6-diethoxy-7-fluoro-1-imino-1,3-dihydro-isoindol-2-yl)-ethanone dihydrochloride, 2-{2-[3-t-butyl-5-(3,4-dihydroxy-pyrrolidin-1-yl)-4-methoxy-phenyl]-2-oxo-ethyl}-6-ethoxy-3-imino-2,3-dihydro-1H-isoindole-5-carboxylic acid methylamide trifluoroacetate; 1-[3-t-butyl-5-(3-hydroxy-4-methoxy-pyrrolidin-1-yl)-4-methoxy-phenyl]-2-(5,6-diethoxy-7-fluoro-1-imino-1,3-dihydro-isoindol-2-yl)-ethanone trifluoroacetate; (3-t-butyl-5-[2-(5,6-diethoxy-7-fluoro-1-imino-1,3-dihydro-isoindol-2-yl)-acetyl]-2-methoxy-phenylamino}-acetonitrile hydrobromide; 1-(3-t-butyl-4-hydroxy-5-morpholino-phenyl)-2-(5,6-diethoxy-7-fluoro-1-imino-1,3-dihydro-isoindol-2-yl)-ethanone hydrochloride; 1-{3-t-butyl-5-[ethyl-(2-hydroxyethyl)-amino]-4-methoxy-phenyl}-2-(5,6-diethoxy-7-fluoro-1-imino-1,3-dihydro-isoindol-2-yl)-acetyl hydrochloride; (4-{3-t-butyl-5-[2-(5,6-diethoxy-7-fluoro-1-imino-1,3-dihydro-isoindol-2-yl)-acetyl]-2-methoxy-phenyl}-piperazin-1-yl)-acetonitrile hydrobromide; 2-(2-{3-t-butyl-5-[(2-hydroxyethyl)-methylamino]-4-methoxy-phenyl}-2-oxo-ethyl)-6-dimethylamino-3-imino-2,3-dihydro-1H-isoindole-5-carboxylic acid methylamide dihydrochloride; 1-{3-t-butyl-5-[(3-hydroxypropyl)-methylamino]-4-methoxy-phenyl}-2-(5,-6-diethoxy-7-fluoro-1-imino-1,3-dihydro-isoindol-2-yl)-ethanone dihydrochloride; 1-{3-t-butyl-5-[(2-hydroxyethyl)-(2-methoxyethyl)-amino]-4-methoxy-phenyl}-2-(5,6-diethoxy-7-fluoro-1-imino-1,3-dihydro-isoindol-2-yl)-ethanone dihydrochloride; 1-[3-t-butyl-5-(3-hydroxy-4-methoxy-pyrrolidin-1-yl)-4-methoxy-phenyl]-2-(5,6-diethoxy-7-fluoro-1-imino-1,3-dihydro-isoindol-2-yl)-ethanone trifluoroacetate; 1-(3-amino-5-t-butyl-4-methoxy-phenyl)-2-(5,6-diethoxy-7-fluoro-1-imino-1,3-dihydro-isoindol-2-yl)-ethanone hydrobromide; 2-[2-(3-t-butyl-5-isopropylamino-4-methoxy-phenyl)-2-oxo-ethyl]-6-ethoxy-3-imino-2,3-dihydro-1H-isoindole-5-carboxylic acid methylamide hydrobromide; 1-[3-(4-acetyl-piperazin-1-yl)-5-t-butyl-4-methoxy-phenyl]-2-(5,6-diethoxy-7-fluoro-1-imino-1,3-dihydro-isoindol-2-yl)-ethanone hydrobromide; 1-[3-t-butyl-5-(3-hydroxy-4-methoxy-pyrrolidin-1-yl)-4-methoxy-phenyl]-2-(5,6-diethoxy-7-fluoro-1-imino-1,3-dihydro-isoindol-2-yl)-ethanone trifluoroacetate; 1-[3-t-butyl-5-(3,4-dimethoxy-pyrrolidin-1-yl)-4-methoxy-phenyl]-2-(5,6-diethoxy-7-fluoro-1-imino-1,3-dihydro-isoindol-2-yl)-ethanone hydrobromide; (4-{3-t-butyl-5-[2-(5,6-diethoxy-7-fluoro-1-imino-1,3-dihydro-isoindol-2-yl)-acetyl]-2-methoxy-phenyl}-piperazin-1-yl)-acetic acid dihydrochloride; 1-{3-t-butyl-5-[4-(2-hydroxy-acetyl)-piperazin-1-yl]-4-methoxy-phenyl}-2-(5,6-diethoxy-7-fluoro-1-imino-1,3-dihydro-isoindol-2-yl)-ethanone hydrobromide; 4-{3-t-butyl-5-[2-(5,6-diethoxy-7-fluoro-1-imino-1,3-dihydro-isoindol-2-yl)-acetyl]-2-methoxy-phenyl}-piperazine-1-carboxylic acid ethylamide hydrobromide; ethyl (4-{3-t-butyl-5-[2-(5,6-diethoxy-7-fluoro-1-imino-1,3-dihydro-isoindol-2-yl)-acetyl]-2-methoxy-phenyl}-piperazin-1-yl)-acetate dihydrochloride; 1-(3-t-butyl-4-methoxy-5-[4-(2-methoxy-acetyl)-piperazin-1-yl]-phenyl}-2-(5,6-diethoxy-7-fluoro-1-imino-1,3-dihydro-isoindol-2-yl)-ethanone hydrobromide; 1-(4-{3-t-butyl-5-[2-(5,6-diethoxy-7-fluoro-1-imino-1,3-dihydro-isoindol-2-yl)-acetyl]-2-methoxy-phenyl}-piperazin-1-yl)-propan-1-one hydrobromide; 1-[3-t-butyl-5-(3-ethoxy-4-hydroxy-pyrrolidin-1-yl)-4-methoxy-phenyl]-2-(5,6-diethoxy-7-fluoro-1-imino-1,3-dihydro-isoindol-2-yl)-ethanone trifluoroacetate; 1-(3,5-di-t-butyl-4-hydroxy-phenyl)-2-(7-imino-2-methyl-5,7-dihydro-pyrrolo[3,4-b]pyridin-6-yl)-ethanone hydrochloride; 1-(3,5-di-t-butyl-4-hydroxy-phenyl)-2-(3-ethoxy-7-imino-2,4-dimethyl-5,7-dihydro-pyrrolo[3,4-b]pyridin-6-yl)-ethanone hydrobromide; 2-[2-(3,5-di-t-butyl-4-hydroxy-phenyl)-2-oxo-ethyl]-6-ethoxy-3-imino-2,3-dihydro-1H-isoindole-5-carboxamide hydrobromide; 2-[2-(8-t-butyl-4-methyl-3,4-dihydro-2H-benzo[1,4]oxazin-6-yl)-2-oxo-ethyl]-6-ethoxy-3-imino-2,3-dihydro-1H-isoindole-5-carboxylic acid methylamide hydrobromide; 6-[2-(3,5-di-t-butyl-4-hydroxy-phenyl)-2-oxo-ethyl]-3-ethoxy-7-imino-6,7-dihydro-5H-pyrrolo[3,4-b]pyridine-2-carboxylic acid methylamide hydrochloride; 1-(3,5-di-t-butyl-4-hydroxy-phenyl)-2-(2-ethyl-7-imino-5,7-dihydro-pyrrolo[3,4-b]pyridin-6-yl)-ethanone hydrobromide; 2-(2-cyclopropyl-7-imino-5,7-dihydro-pyrrolo[3,4-b pyridin-6-yl)-ethanone)-1-(3,5-di-t-butyl-4-hydroxy-phenyl)-ethanone hydrobromide; 2-[2-(8-t-butyl-3-oxo-3,4-dihydro-2H-benzo[1,4]oxazin-6-yl)-2-oxo-ethyl]-6-ethoxy-3-imino-2,3-dihydro-1H-isoindole-5-carboxylic acid methylamide hydrobromide; and 1-(3-t-butyl-5-isopropylamino-4-methoxy-phenyl)-2-(5,6-diethoxy-7-fluoro-1-imino-1,3-dihydro-isoindol-2-yl)-ethanone hydrobromide.

Peptide PAR1 antagonists are also known, including the peptide

MSRPACPN (SEQ ID NO: 1), described in Doorbar & Winter (1994), supra.

Other peptide antagonists include AFLARAA (SEQ ID NO: 2), described in Pakala et al. (2001), supra.

Other non-peptide PAR1 antagonists include eryloside F, a penasterol disaccharide, described in Stead et al. (2000), supra.

U.S. Pat. No. 6,017,890 to Hoekstra et al. disclosed azole peptidomimetic-based PAR1 antagonists. These compounds are of the general formula shown below as Formula (XXII).

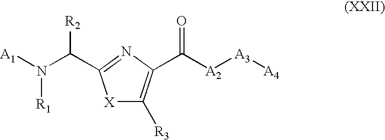

wherein: (1) A₁ is an amino acid residue selected from Sar, Gly, H is, H is (CH₂ Ph), Ile, Ser, Thr, β-Ala, or Ala; A₁ may also be a C₂-C₆-acyl group such as, for example, acetyl, propionyl or butyryl, or a C₁-C₈-alkyl group such as, for example, methyl, ethyl, propyl or butyl (2) A₂ is an alkyl amino acid residue selected from Cha, Leu, Ile, Asp, and Glu or an amino alkyl amino acid residue such as Lys, H is, Orn, homoArg and Arg; (3) A₃ is an amino alkyl amino acid residue selected from Lys, His, Orn, Arg and homoArg; (4) A₄ is an arylalkyl residue selected from Phe and Tyr or an aralkylamino group such as benzylamino or a phenethylamino group; (5) R₁ is selected from H or alkyl; (6) R₂ is an aryl, substituted aryl, heteroaryl, substituted heteroaryl, aralkyl or substituted aralkyl group, however, R₂ is preferably aralkyl; (7) R₃ is selected from H or alkyl; and (8) X is selected from S, O, or NR₄, wherein R₄ is selected from H or alkyl; and the pharmaceutically acceptable salts thereof. Particularly preferred compounds include 2-[1(S)-sarcosineamido-2-(4-fluorophenyl)ethyl]oxazole-4-carboxy-cyclohexyl alanyl-arginine benzylamide; 2-[1(S)-β-alanineamido-2-(4-fluorophenyl)ethyl]oxazole-4-carboxy-cyclohexylalanyl-arginine benzylamide; and 2-[1(S)-sarcosineamido-2-(4-fluorophenyl)ethyl]thiazole-4-carboxy-cyclohexylalanyl-arginine phenethylamide.

Peptide derivative PAR1 antagonists are described in PCT Patent Application Publication No. WO 94/03479 by Scarborough. The antagonists are compounds of Formula (XXIII), below, in which R¹ and R³ are amide linkages, N-alkylamide linkages, or isosteric replacements of such linkages; R² is either a neutral amino acid side chain or a hydrophobic radical; R⁴ is a hydrophobic radical; R⁵ is CO, CH², or SO; X is either; (1) a moiety of Formula (XXIV), below, in which R⁶ and R⁷ are the same or different and are hydrogen, alkyl, (cycloalkyl)alkyl, alkoxyalkyl, alkylthioalkyl, or aralkyl; or (2) a hydrophobic residue containing at least one aryl moiety; and R⁸ is a hydrophobic radical, or is a hydrophobic residue, typically containing an alkyl moiety. Y is alkoxy, hydroxyl, amino, alkylamino, or dialkylamino, in which any of the alkyl groups can be substituted with a basic moiety, Z is an amino acid or peptide residue; and m is 0 or 1.

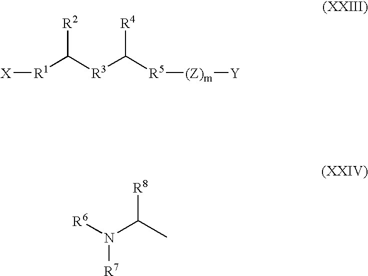

In some embodiments, a peptide or peptidomimetic based PAR1 antagonist compound is used in the therapeutic and prophylactic applications of the invention. Such PAR1 antagonists are well known in the art. For example, Andrade-Gordon et al. (1999), supra, disclosed design, synthesis, and biological characterization of peptidomimetic based PAR1 antagonists, e.g., RWJ-56110. An analogue of RWJ-56110 in which the substituted indole ring of RWJ-56110 is replaced by an identically substituted indazole ring (in other words, a —CH group in RWJ-56110 is replaced by a —N group) is RWJ-58259, disclosed in Barry et al. (2006), supra, and Damiano et al. (2003), supra. Many other peptide or peptidomimetic based antagonists of PAR1 are also described in the art. See, e.g., Hoekstra et al. (1998), supra; Bernatowicz et al. (1996), supra; Pakala et al. (2001), supra; Elliott et al. (1999), supra; Zhang et al. (2001), supra; Ruda et al. (1988), supra; Doorbar & Winter (1994), supra; Maryanoff et al. (2003), supra; Seiler & Bernatowicz (2003), supra; and Damiano et al. (2003), supra.

One specific example of a peptidomimetic PAR1 antagonist that can be readily employed in the present invention is RWJ-56110 [(αS)—N-[(1S)-3-amino-1-[[(phenylmethyl)amino]carbonyl]propyl]-α-[[t[1-(2,6-dichlorophenyl)methyl]-3-(1-pyrrolidinylmethyl)-1H-indol-6-yl]amino]carbonyl]amino]-3,4-difluorobenzenepropanamide]. This compound is potent, selective PAR-1 antagonist which does not affect PAR2, PAR3, or PAR4 or inhibit thrombin activity. It binds to PAR-1 and interferes with calcium mobilization and cellular functions associated with PAR-1.

Some other embodiments of the invention employ a pepducin antagonist of PAR1 which is a chimeric polypeptide comprising the third intracellular loop (i3 loop) of PAR1 attached to a lipid moiety. Preparation and activities of such compounds are described in L. Covic et al., “Pepducin-Based Intervention of Thrombin-Receptor Signaling and Systemic Platelet Activation,” Nat. Med. 8:1161-1165 (2002) (“Covic et al. (2002”); and U.S. Pat. No. 6,864,229 to Kuliopulos et al., both of which are hereby incorporated by this reference. Among the pepducins described in these references is pal-RCLSSSAVANRS (SEQ ID NO: 3), which has a palmitoylated arginine residue at the amino-terminus of a 12-amino-acid peptide derived from the i3 loop of PAR1. Another of these pepducins described in these references is pal-KKSRALF (SEQ ID NO: 4), which has a palmitoylated lysine residue at the amino-terminus of a 7-amino acid peptide derived from a short transmembrane loop of PAR1.

In general, pepducins usable in methods according to the present invention comprise: (1) a first domain that includes either extracellular or intracellular portions of a G protein coupled receptor (GPCR) such as, in this case, PAR1; and (2) at least a second domain, attached to the first domain. The second domain is a hydrophobic moiety which is either naturally or non-naturally occurring. Furthermore, the first domain does not comprise a native extracellular ligand of the GPCR. The second domain can be attached at one end or at an internal position of the first domain. If there is both a second and a third domain, they can be attached, interchangeably, at both ends, or at internal positions within the first domain. Typically, in pepducins, the hydrophobic moiety is either a lipid moiety or an amino acid moiety. For example, the hydrophobic moiety can be selected from the group consisting of: phospholipids, steroids, sphingosines, ceramides, octyl-glycine, 2-cyclohexylalanine, and benzolylphenylalanine. Alternatively, the hydrophobic moiety can be a hydrophobic substituent attached to an amino acid in the first domain. The hydrophobic substituent can be selected from the group consisting of propionoyl (C₃); butanoyl (C₄); pentanoyl (C₅), caproyl (C₆); heptanoyl (C₇); capryloyl (C₈); nonanoyl (C₉); capryl (C₁₀); undecanoyl (C₁₁); lauroyl (C₁₂); tridecanoyl (C₁₃); myristoyl (C₁₄); pentadecanoyl (C₁₅); palmitoyl (C₁₆); phytanoyl (tetramethyl-C₁₆); heptadecanoyl (C₁₇); stearoyl (C₁₈); nonadecanoyl (C₁₉); arachidoyl (C₂₀); heniecosanoyl (C₂₁); behenoyl (C₂₂); trucisanoyl (C₂₃); and lignoceroyl (C₂₄). The hydrophobic moiety can be attached to the first domain said hydrophobic moiety with amide bonds, sulfhydryls, amines, alcohols, phenolic groups, or carbon-carbon bonds. In another alternative, the hydrophobic moiety can be transmembrane domain 5 of the GPCR, in this case, transmembrane domain 5 of PAR1, or a fragment thereof.

In some embodiments, the therapeutic methods of the invention employ a non-peptide small molecule antagonist of PAR1. Many specific small molecule PAR1 antagonists are disclosed in the art. For example, Nantermet et al. (2002) reported aminoisoxazole derived PAR1 antagonist compounds. Pyrroloquinazoline, benzimidazole, and himbacine based PAR1 antagonist compounds are described in Chalackamannil et al. (2003), supra. Selnick et al. (2003), supra, disclosed small molecule PAR1 antagonist compounds which do not inhibit the proteolytic effects of thrombin but rather interfere with the intramolecular binding of the tethered ligand to PAR1, specifically to the transmembrane portion of the thrombin receptor. Many other non-peptide PAR1 antagonist compounds have also been disclosed in the art. See, e.g., Kato et al. (1999), supra; H.-S. Ahn et al., “Structure-Activity Relationships of Pyrroloquinazolines as Thrombin Receptor Antagonists,” Bioorg. Med. Chem. Lett. 9:2073-2078 (1999) (“Ahn et al. (1999)”); Nantermet et al. (2002), supra, J. C. Barrow et al., “Discovery and Initial Structure-Activity Relationships of Trisubstituted Ureas as Thrombin Receptor (PAR-1) Antagonists,” Bioorg. Med. Chem. Lett. 11: 2691-2696 (2001) (“Barrow et al. (2001)”); Ahn et al. (2000), supra; P. Stead et al., “Eryloside F, a Novel Penasterol Disaccharide Possessing Potent Thrombin Receptor Antagonist Activity,” Bioorg Med. Chem. Lett. 19: 661-664 (2000) (“Stead et al. (2000)”); and S. Chackalamannil et al., “Potent, Low Molecular Weight Thrombin Receptor Antagonists,” Bioorg. Med. Chem. Lett. 11:2851-2853 (2001).

A specific non-peptide PAR1 selective antagonist compound suitable for practicing the present invention is SCH-79797. This compound, (N-3-cyclopropyl-7-{[4-(1-methylethyl)phenyl]methyl}-7H-pyrrolo[3,2-f]quinazoline-1,3-diamine), is a PAR1 selective antagonist. It is commercially available from Tocris Bioscience (Ellisville, Mo.).

Compounds suitable for use in methods and compositions according to the present invention also include salts, solvates, analogues, congeners, bioisosteres, hydrolysis products, metabolites, precursors, and prodrugs of the peptides, peptidomimetics, and other small molecule PAR1 antagonists described above where such salts, solvates, analogues, congeners, bioisosteres, hydrolysis products, metabolites, precursors, and prodrugs have activity equivalent to the peptides, peptidomimetics, and other small molecule PAR1 antagonists.

In the case of salts, it is well known that organic compounds, including compounds having activities suitable for methods according to the present invention, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound or analogue includes such salt forms as occur at physiological pH or at the pH of a pharmaceutical composition unless specifically excluded.

Similarly, prodrug esters can be formed by reaction of either a carboxyl or a hydroxyl group on compounds or analogues suitable for methods according to the present invention with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art. For example prodrugs can include amides prepared by reaction of the parent acid compound with a suitable amine. In some cases it is desirable to prepare double ester type prodrugs such as (acyloxy) alkyl esters or ((alkoxycarbonyl)oxy)alkyl esters. Suitable esters as prodrugs include, but are not necessarily limited to, methyl, ethyl, propyl, isopropyl, n-butyl, isobutyl, tert-butyl, morpholinoethyl, and N,N-diethylglycolamido. Methyl ester prodrugs may be prepared by reaction of the acid form of a compound having a suitable carboxylic acid group in a medium such as methanol with an acid or base esterification catalyst (e.g., NaOH, H₂ SO₄). Ethyl ester prodrugs are prepared in similar fashion using ethanol in place of methanol. Morpholinylethyl ester prodrugs may be prepared by reaction of the sodium salt of a suitable compound (in a medium such as dimethylformamide) with 4-(2-chloroethyl)morphine hydrochloride (available from Aldrich Chemical Co., Milwaukee, Wis. USA.

The use of prodrugs is further described in S. C. Gad, ed., “Drug Discovery Handbook” (Wiley-Interscience, Hoboken, N.J., 2005), ch. 17, pp. 733-796, incorporated herein by this reference, which describes prodrug formulation and use. Typically, prodrugs are formed by covalently linking a promoiety to a nucleophilic carboxyl, phosphate, phosphonate, hydroxyl, or amino group on the parent drug molecule via a labile linkage that can be cleaved enzymatically, or, in some cases, nonenzymatically. Enzymes that are most commonly targeted for cleavage of a labile prodrug linkage include various esterases and alkaline phosphatases, as well as other enzymes, such as aminoacylases, cystein conjugate β-lyase, γ-glutamyltransferases, dipeptidases, aminopeptidases, carboxypeptidases, oxoprolinase, β-glucuronidase, and azoreductase. In some cases, the enzymes being targeted by prodrugs occur only in specific tissues or organs, so such prodrugs can be appropriate for organ- or tissue-targeted drug delivery. In some cases, however, prodrugs are used without a promoiety. In these cases, the active drug is generated from the prodrug by an oxidative reaction, a reaction mediated by a kinase enzyme, or other complex transformation. In some cases, lipophilic prodrugs can be generated from drugs containing a hydroxyl group by esterification with a carboxylic acid that has a non-polar side chain, such as a long-chain fatty acid. Prodrugs can be used to achieve improved drug absorption, to achieve drug delivery to specific tissues or organs, to improve aqueous solubility, to prolong duration of action, to reduce side effects, and to achieve improved drug targeting.

Specifically, prodrugs have been used for peptide drugs to improve their aqueous solubility, such as O-acyl peptide isomers that can spontaneously revert to their normal N-acyl forms under physiological conditions through the N,O-acyl migration reaction. Other prodrugs have been developed to protect against rapid enzymatic degradation, such as derivatization of the C-terminal carboxyl group with a highly lipophilic 1,3-dipalmitoyl acetyl glycerol promoiety. Peptides possessing an α-aminoamide moiety can be condensed with aldehydes or ketones to form 4-imidazolidone prodrugs that are spontaneously hydrolyzed in aqueous solutions. Peptides containing histidine can also be modified on the imidazole group to form N-alkoxycarbonyl prodrugs. Prodrugs can also be formed by forming an ester with hydroxyl groups on amino acid side chains, such as O-pivaloyl esters with the hydroxyl group on tyrosine. Another approach is bioreversible cyclization by linking the N-terminal amino group and the C-terminal carboxyl group through an enzyme-labile promoiety to achieve increased membrane penetration. Other routes for prodrug formation are known in the art.

Pharmaceutically acceptable salts include acid salts such as hydrochlorides, hydrobromides, hydroiodides, sulfates, phosphates, fumarates, maleates, acetates, citrates, lactates, tartrates, sulfamates, malonate, succinate, tartrate, methanesulfonates, ethanesulfonates, benzenesulfonates, p-toluenesulfonates, cyclohexylsulfamates, quinates, formates, cinnamates, picrates, and other suitable salts. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts also include salts with bases such as alkali metal salts such as sodium or potassium, as well as pyridine salts, ammonium salts, piperazine salts, diethylamine salts, nicotinamide salts, calcium salts, magnesium salts, zinc salts, lithium salts, methylamino salts, triethylamino salts, dimethylamino salts, and tris(hydroxymethyl)aminomethane salts.

PAR1 antagonists suitable for practicing the present invention also include antagonist PAR1 antibodies. These anti-PAR1 agents are capable of antagonizing PAR1 mediated signaling activities, e.g., PAR1 mediated interleukin secretion. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, N.Y., 1998; Kohler & Milstein, Nature 256:495-497 (1975); Kozbor et al., Immunology Today 4:72 (1983); and Cole et al., pp. 77-96 in Monoclonal Antibodies and Cancer Therapy, 1985. In addition, specific PAR1 antagonist antibodies have been disclosed in the art. See, e.g., R. R. Vassallo, Jr. et al. “Structure-Function Relationships in the Activation of Platelet Thrombin Receptors by Receptor-Derived Peptides,” J. Biol. Chem. 267:6081-6085 (1992) (“Vassallo, Jr. et al. (1992”)); L. F. Brass et al., “Structure and Function of the Human Platelet Thrombin Receptor,” J. Biol. Chem. 267: 13795-13798 (1992) (“Brass et al. (1992)”); and R. Kaufmann et al., “Investigation of PAR-1-Type Thrombin Receptors in Rat Glioma C6 Cells with a Novel Monoclonal Anti-PAR-1 Antibody (Mab COR7-6H9), J. Neurocytol. 27:661-666 (1998) (“Kaufmann et al. (1998)”), both of which are incorporated herein by this reference.

In Brass et al. (1992), monoclonal antibodies were prepared against the immunogen SFLLRNPNDKYEPF (SEQ ID NO: 5) which represents residues 42-55 of human PAR1. These monoclonal antibodies were prepared by standard techniques, beginning with the immunization of mice with the immunogen SFLLRNPNDKYEPF (SEQ ID NO: 5) conjugated to keyhole limpet hemocyanin (KLH). These monoclonal antibodies include: (1) a monoclonal antibody designated ATAP2 in Brass et al. (1992), which binds to a first fragment of the immunogen, specifically SFLLRNPND (SEQ ID NO: 6); (2) a monoclonal antibody designated ATAP120 in Brass et al. (1992), which binds to a second fragment of the immunogen, specifically NPNDKYEPF (SEQ ID NO: 7); and a monoclonal antibody designated ATAP138 in Brass et al., which also binds to NPNDKYEPF (SEQ ID NO: 7). Additionally, monoclonal antibodies usable in compositions and methods according to the present invention include monoclonal antibodies that specifically bind either or both of SFLLRNPND (SEQ ID NO: 6) or NPNDKYEPF (SEQ ID NO: 6) such that these antibodies have an affinity for either or both of SFLLRNPND (SEQ ID NO: 6) or NPNDKYEPF (SEQ ID NO: 7) that is at least 80% as great as any of ATAP2, ATAP20, or ATAP138, as measured by the reciprocal of the dissociation constant for the antibody-antigen complex. Additionally, monoclonal antibodies usable in compositions and methods according to the present invention include monoclonal antibodies that have complementary-determining regions that are identical to those of ATAP2, ATAP20, or ATAP138. Additionally, monoclonal antibodies usable in compositions and methods according to the present invention include monoclonal antibodies that have complementary-determining regions that are identical to the monoclonal antibodies described above that specifically bind either or both of SFLLRNPND (SEQ ID NO: 6) or NPNDKYEPF (SEQ ID NO: 7) or such that these antibodies have an affinity for either or both of SFLLRNPND (SEQ ID NO: 6 or NPNDKYEPF (SEQ ID NO: 7) that is at least 80% as great as any of ATAP2, ATAP20, or ATAP138.

Kaufmann et al. (1998) described monoclonal antibodies to rat PAR1 receptor that were prepared by using a peptide with the sequence GRAVYLNKSRFPPMPPPPFISEDASG (SEQ ID NO: 8). This sequence is described as being below the thrombin cleavage site for the receptor. Analogous antibodies can be prepared against the corresponding region of human PAR1 receptor. In general, antibodies according to the present invention can be of any class, such as IgG, IgA, IgD, IgE, IgM, or IgY, although IgG antibodies are typically preferred. Antibodies can be of any mammalian or avian origin, including human, murine (mouse or rat), donkey, sheep, goat, rabbit, camel, horse, or chicken. In some alternatives, the antibodies can be bispecific. The antibodies can be modified by the covalent attachment of any type of molecule to the antibody. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, or other modifications known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., “Antibodies: A Laboratory Manual”, (Cold Spring Harbor Laboratory Press, 2nd ed. 1988); Hammerling, et al., in: Monoclonal Antibodies and T-Cell Hybridomas 563-681 (Elsevier, N.Y., 1981), or by other standard methods known in the art. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. For example, suitable antibodies can be produced by phage display or other techniques. Additionally, and not by way of limitation, human antibodies can be made by a variety of techniques, including phage display methods using antibody libraries derived from human immunoglobulin sequences and by the use of transgenic mice that are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. The antibodies can also be produced by expression of polynucleotides encoding these antibodies. Additionally, antibodies according to the present invention can be fused to marker sequences, such as a peptide tag to facilitate purification; a suitable tag is a hexahistidine tag. The antibodies can also be conjugated to a diagnostic or therapeutic agent by methods known in the art. Techniques for preparing such conjugates are well known in the art.

Other methods of preparing these monoclonal antibodies, as well as chimeric antibodies, humanized antibodies, and single-chain antibodies, are known in the art.

In addition to compounds which inhibit or suppress PAR1 biochemical or signaling activities, compounds which are capable of suppressing PAR1 expression or down-regulating PAR1 cellular levels can also be used in the practice of the present invention. Suppression of PAR1 expression or down-regulation of its cellular level refers to a decrease in or an absence of PAR1 expression in an examined cell (e.g., a cell which has been contacted with a PAR1 antagonist compound), as compared to PAR1 in a control cell (a cell not treated with the PAR1 antagonist compound). PAR1 level or expression can be decreased or reduced by at least about 10% (e.g., by 20%, 30%, 40%, 50%, 60%, 70%, 80%, or 90%), as compared to PAR1 level or expression in the control cell.

As indicated above, suppression of expression or down-regulation of PAR1 cellular levels can be carried out at either the level of transcription of the gene for PAR1 into mRNA or the translation of mRNA for PAR1 into the corresponding protein.

In some embodiments, inhibitory nucleotides are used to antagonize PAR1 mediated cardiac remodeling or other effects of PAR1 by suppressing PAR1 expression. These include short interfering RNA (siRNA), microRNA (miRNA), and synthetic hairpin RNA (shRNA), anti-sense nucleic acids, or complementary DNA (cDNA). In some preferred embodiments, a siRNA targeting PAR1 expression is used. Interference with the function and expression of endogenous genes by double-stranded RNA such as siRNA has been shown in various organisms. See, e.g., A. Fire et al., “Potent and Specific Genetic Interference by Double-Stranded RNA in Caenorhabditis elegans,” Nature 391:806-811 (1998); J. R. Kennerdell & R. W. Carthew, “Use of dsDNA-Mediated Genetic Interference to Demonstrate that frizzled and frizzled 2 Act in the Wingless Pathway,” Cell 95:1017-1026 (1998); F. Wianni & M. Zernicka-Goetz, “Specific Interference with Gene Function by Double-Stranded RNA in Early Mouse Development,” Nat. Cell Biol. 2:70-75 (2000). siRNAs can include hairpin loops comprising self-complementary sequences or double stranded sequences. siRNAs typically have fewer than 100 base pairs and can be, e.g., about 30 bps or shorter, and can be made by approaches known in the art, including the use of complementary DNA strands or synthetic approaches. Such double-stranded RNA can be synthesized by in vitro transcription of single-stranded RNA read from both directions of a template and in vitro annealing of sense and antisense RNA strands. Double-stranded RNA targeting PAR1 can also be synthesized from a cDNA vector construct in which a PAR1 gene (e.g., human PAR1 gene) is cloned in opposing orientations separated by an inverted repeat. Following cell transfection, the RNA is transcribed and the complementary strands reanneal. Double-stranded RNA targeting the PAR1 gene can be introduced into a cell (e.g., a tumor cell) by transfection of an appropriate construct. Typically, RNA interference mediated by siRNA, miRNA, or shRNA is mediated at the level of translation; in other words, these interfering RNA molecules prevent translation of the corresponding mRNA molecules and lead to their degradation. It is also possible that RNA interference may also operate at the level of transcription, blocking transcription of the regions of the genome corresponding to these interfering RNA molecules. The structure and function of these interfering RNA molecules are well known in the art and are described, for example, in R. F. Gesteland et al., eds, “The RNA World” (3^{rd} ed, Cold Spring Harbor Laboratory Press, Cold Spring Harbor, N.Y., 2006), pp. 535-565, incorporated herein by this reference.

For these approaches, cloning into vectors and transfection methods are also well known in the art and are described, for example, in J. Sambrook & D. R. Russell, “Molecular Cloning: A Laboratory Manual” (3^{rd} ed., Cold Spring Harbor Laboratory Press, Cold Spring Harbor, 2001), incorporated herein by this reference.

In addition to double stranded RNAs, other nucleic acid agents targeting PAR1 can also be employed in the methods of the present invention, e.g., antisense nucleic acids. Antisense nucleic acids are DNA or RNA molecules that are complementary to at least a portion of a specific target mRNA molecule. In the cell, the single stranded antisense molecule hybridizes to that mRNA, forming a double stranded molecule. The cell does not translate an mRNA in this double-stranded form. Therefore, antisense nucleic acids interfere with the translation of mRNA into protein, and, thus, with the expression of a gene that is transcribed into that mRNA. Antisense methods have been used to inhibit the expression of many genes in vitro. See, e.g., C. J. Marcus-Sekura, “Techniques for Using Antisense Oligodeoxyribonucleotides to Study Gene Expression,” Anal. Biochem. 172:289-295 (1988); J. E. Hambor et al., “Use of an Epstein-Barr Virus Episomal Replicon for Anti-Sense RNA-Mediated Gene Inhibition in a Human Cytotoxic T-Cell Clone,” Proc. Natl. Acad. Sci. U.S.A. 85:4010-4014 (1988); H Arima et al., “Specific Inhibition of Interleukin-10 Production in Murine Macrophage-Like Cells by Phosphorothioate Antisense Oligonucleotides,” Antisense Nucl. Acid Drug Dev. 8:319-327 (1998); and W.-F. Hou et al., “Effect of Antisense Oligodeoxynucleotides Directed to Individual Calmodulin Gene Transcripts on the Proliferation and Differentiation of PC12 Cells,” Antisense Nucl. Acid Drug Dev. 8:295-308 (1998), all incorporated herein by this reference. Antisense technology is described further in C. Lichtenstein & W. Nellen, eds., “Antisense Technology: A Practical Approach” (IRL Press, Oxford, 1997), incorporated herein by this reference.

PAR1 polynucleotide sequences from human and many other mammals have all been delineated in the art. For example, human PAR1 cDNA sequence (NM_{—}001992) was reported in T.-K. H. Vu et al., “Molecular Cloning of a Functional Thrombin Receptor Reveals a Novel Proteolytic Mechanism of Receptor Activation,” Cell 64:1057-1068 (1991), incorporated herein by this reference. Based on the known sequences, inhibitory nucleotides (e.g., siRNA, miRNA, or shRNA) targeting PAR1 can be readily synthesized using methods well known in the art. Exemplary siRNAs according to the invention could have up to 29 bps, 25 bps, 22 bps, 21 bps, 20 bps, 15 bps, 10 bps, 5 bps or any integral number of base pairs between these numbers. Tools for designing optimal inhibitory siRNAs include that available from DNAengine Inc. (Seattle, Wash.) and Ambion, Inc. (Austin, Tex.). Specific PAR1 inhibitory nucleotides and their use in down-regulating PAR1 expression have also been disclosed in the art, e.g., Q. Fang et al., “Thrombin Induces Collagen Gel Contraction Partially Through PAR1 Activation and PKC-ε,” Eur. Respir. J. 24:918-924 (2004); and Y.-J. Yin et al., “Mammary Gland Tissue Targeted Overexpression of Human Protease-Activated Receptor 1 Reveals a Novel Link to β-Catenin Stabilization,” Cancer Res. 66:5224-5233 (2006), both incorporated herein by this reference.

Accordingly, one aspect of the present invention is a method for treating or preventing a cardiac dysfunction in a subject, comprising administering to a subject having or being at risk of developing a cardiomyopathy a therapeutically effective amount of an antagonist of protease activated receptor 1 (PAR1), thereby treating or preventing cardiac dysfunction in the subject.

A number of potential routes of administration are well known in the art. Local administration of PAR1 antagonists is desired in order to achieve the intended therapeutic effect. Many methods of localized delivery of therapeutic agents or formulations can be used in the practice of the invention.

For example, local administration of a PAR1 antagonist to the desired cardiac muscle in a subject can be accomplished by a percutaneous route, by therapeutic cardiac catheterization, by intrapericardial injection or infusion, or by direct intracardiac muscle injection. Suitable methods also include any other routes which allow the therapeutic agent to be applied locally to the heart. For example, the therapeutic agent may be applied from the bloodstream, by being placed directly in the heart through the coronary arteries or veins onto the heart surface, or through the ventricular or atrial walls and onto the heart surface. The therapeutic agent may also be applied through direct application during extensive surgical field exposure, or through direct application during minimally invasive exposure, e.g., through a pericardial window or heart port. Other routes of administration are also known in the art.

Another aspect of the present invention is a method for reducing the size or extent of infarct after thrombolysis of a clot and reperfusion of cardiac tissue. This aspect comprises administering to a subject in whom infarct has occurred a therapeutically effective amount of an antagonist of PAR1, thereby reducing the size or extent of infarct in the subject. A recent study using an antagonist of PAR1 has shown a decrease in infarct size (J. L. Strande et al., “SCH 79797, A Selective PAR1 Antagonist, Limits Myocardial Ischemia/Reperfusion Injury in Rat Hearts,” Basic Res. Cardiol. 102; 350-358 (2007), incorporated herein by this reference). This is further described in Meadows & Bhatt (T. A. Meadows & D. L. Bhatt, “Clinical Aspects of Platelet Inhibitors and Thrombus Formation,” Circ. Res. 100: 1261-1275 (2007), incorporated herein by this reference).

Another aspect of the present invention involves the use of an antibody that can block the signaling activity of Tissue Factor (TF). As shown below in Example 7, TF interacts with PAR1 such that blockage of TF signaling activity further reduces the occurrence of cardiac hypertrophy and other complications resulting from PAR1 signaling activity following myocardial infarction. In order to block the signaling activity of TF without unduly interfering with necessary hemostatic activity, it is preferred to block the signaling activity of by using an antibody, such as a monoclonal antibody, that specifically blocks the signaling activity of TF without blocking its procoagulant activity. Ahamed et al. (J. Ahamed et al., “Disulfide Isomerization Switches Tissue Factor from Coagulation to Cell Signaling,” Proc. Natl. Acad. Sci. U.S.A. 103: 13932-13937 (2007)) has shown that TF occurs in two forms, a procoagulant form and a signaling form. The interconversion between the two forms is controlled by the state of a potentially formed disulfide bond between Cys¹⁸⁶ and Cys²⁰⁹ of TF. If this disulfide bond links the two cysteine residues, TF mediates coagulation activation. If this disulfide bond is not formed, so that Cys¹⁸⁶ and Cys²⁰⁹ are present as unlinked cysteine residues with —SH groups, then TF is involved in signaling, particularly with Factor VIIa. A TF mutant (TFC209A) that mutates Cys²⁰⁹ to alanine, thus preventing the possibility of disulfide bond formation with Cys¹⁸⁶, retains TF/Factor VIIa signaling activity, but has reduced affinity for Factor VIIa and thus is far less active in coagulation than the TF form with the disulfide bond between Cys¹⁸⁶ and Cys²⁰⁹. The interconversion between the procoagulant form and the signaling form of TF is controlled by the a disulfide exchange reaction catalyzed by extracellular protein disulfide isomerase (PDI). A monoclonal antibody that can bind to the signaling form of TF is designated mAb 10H10 This monoclonal antibody blocks signaling activity of TF without significantly blocking coagulation activation.

Along these lines, monoclonal antibody mAb 10H10 blocked tumor growth in mice (H. H. Versteeg et al., “Inhibition of Tissue Factor Signaling Suppresses Tumor Growth,” Blood 111: 190-199 (2008)).

Accordingly, another aspect of the present invention is a method for treating or preventing a cardiac dysfunction in a subject, comprising administering to a subject having or being at risk of developing a cardiomyopathy:

(1) a therapeutically effective amount of an antagonist of protease activated receptor 1 (PAR1); and

(2) a therapeutically effective amount of a monoclonal antibody specifically binding to TF that specifically blocks signaling activity of TF without substantially interfering with coagulation activity of TF, thereby treating or preventing cardiac dysfunction in the subject.

In this aspect of methods according to the present invention, the monoclonal antibody specifically binding to TF can be mAb 10H10. Alternatively, the monoclonal antibody can be a monoclonal antibody that specifically binds the same antigen bound by mAb 10H10 such that the antibody has an affinity for the antigen that is at least 80% as great as that of mAb 10H10 as measured by the reciprocal of the dissociation constant for the antibody-antigen complex. Additionally, the monoclonal antibody can be a monoclonal antibody that has complementarity-determining regions that are identical to those of mAb10H10.

In these methods according to the present invention, the PAR1 antagonist can be any PAR1 antagonist described above.

Another aspect of the present invention is based on an apparent interaction between the activities of PAR1 and PAR2. This is shown in Example 8, below, where it is demonstrated that PAR2 knockout mice have a reduced infarct size as compared with wild-type mice. Therefore, inhibition of PAR2 can further potentiate the effect of PAR1 inhibition and increase the protection provided by the use PAR1 antagonists. Therefore, this aspect of the present invention is a method for treating or preventing a cardiac dysfunction in a subject, comprising administering to a subject having or being at risk of developing a cardiomyopathy:

(1) a therapeutically effective amount of an antagonist of protease activated receptor 1 (PAR1); and

(2) a therapeutically effective amount of an antagonist of protease activated receptor 2 (PAR2). Suitable PAR2 antagonists are known in the art, and include the peptide FSLLRY—NH₂ (SEQ ID NO: 9) (S. Wilson et al., “The Membrane-Anchored Serine Protease, TMPRSS2, Activates PAR-2 in Prostate Cancer Cells,” Biochem. J. 388: 967-972 (2005)); LSIGRL (SEQ ID NO: 10) (U.S. Patent Application Publication No. 2006/0104944 by Mousa); and N¹-3-methylbutyryl-N⁴-6-aminohexanoyl-piperazine (ENMD-1068) (E. B. Kelso et al., “Therapeutic Promise of Proteinase-Activated Receptor-2 Antagonism in Joint Inflammation,” J. Pharmacol. Exp. Ther. 316: 1017-1024 (2006)).

Alternatively, the antagonist of PAR2 can be an antibody specifically binding PAR2. Such antibodies can be prepared in much the same way as anti-PAR1 antibodies, described above.

In another alternative, the PAR1 antagonist can be administered together with another cardiovascular agent. For example, the cardiovascular agent can be selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACE/NEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIa inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibitors, renin inhibitors, inhibitors of adenosine diphosphate (“ADP”)-induced platelet aggregation, and NHE-1 inhibitors.

Calcium channel blockers can include, but are not limited to, amlodipine besilate, felodipine, diltiazem, nifedipine, nicardipine, nisoldipine, bepridil, and verapamil.

Statins can include, but are not limited to, atorvastatin, fluvastatin, lovastatin, pitavastatin, pravastatin, rosuvastatin, and simvastatin.

Cholesterol absorption inhibitors can include, but are not limited to, ezetimibe and AZD4121.

Cholesteryl ester transfer protein (“CETP”) inhibitors, can include, but are not limited to, torcetrapib.

Low molecular weight heparins can include, but are not limited to, dalteparin sodium, ardeparin, certoparin, enoxaparin, parnaparin, tinzaparin, reviparin, nadroparin, warfarin, ximelagatran, and fondaparin.

Antiarrythmic agents can include, but are not limited to, dofetilide and ibutilide fumarate, metoprolol, metoprolol tartrate, propranolol, atenolol, ajmaline, disopyramide, prajmaline, procainamide, quinidine, sparteine, aprindine, lidocaine, mexiletine, tocamide, encamide, flecamide, lorcamide, moricizine, propafenone, acebutolol, pindolol, amiodarone, bretylium tosylate, bunaftine, dofetilide, sotalol, adenosine, atropine and digoxin.

Alpha-adrenergic agonists can include, but are not limited, to doxazosin mesylate, terazoson, and prazosin.

Beta-adrenergic blocking agents can include, but are not limited to, carvedilol, propranolol, timolol, nadolol, atenolol, metoprolol, bisoprolol, nebivolol, betaxolol, acebutolol, and bisoprolol.

Aldosterone antagonists can include, but are not limited to, eplerenone, and spironolactone.

Angiotensin-converting enzyme (“ACE”) inhibitors can include, but are not limited to, moexipril, quinapril hydrochloride, ramipril, lisinopril, benazepril hydrochloride, enalapril, captopril, spirapril, perindopril, fosinopril, and trandolapril.

ACE/NEP inhibitors can include, but are not limited to, ramipril.

Angiotensin II receptor blockers (“ARBs”) can include, but are not limited to, olmesartan medoxomil, candesartan, valsartan, telmisartan, irbesartan, losartan, and eprosartan.

Endothelin antagonists can include, but are not limited to, tezosentan, bosentan, and sitaxsentan sodium.

Neutral endopeptidase inhibitors can include, but are not limited to, candoxatril and ecadotril.

Phosphodiesterase inhibitors can include, but are not limited to, milrinoone, theophylline, vinpocetine, EHNA (erythro-9-(2-hydroxy-3-nonyl)adenine), sildenafil citrate, and tadalafil.

Fibrinolytics can include, but are not limited to, reteplase, alteplase, and tenecteplase.

GP IIb/IIIa antagonists can include, but are not limited to, integrillin, abciximab, and tirofiban.

Direct thrombin inhibitors can include, but are not limited to, ximelagatran and AZD0837.

Indirect thrombin inhibitors can include, but are not limited to, odeparcil.

Direct Factor Xₐ inhibitors can include, but are not limited to, fondaparinux sodium, apixaban, razaxaban, rivaroxaban (BAY 59-7939), KFA-1982, DX-9065a, AVE3247, otamixaban (XRP0673), AVE6324 and SAR377142.

Indirect Factor Xₐ inhibitors can include, but are not limited to, idraparinux (long-acting pentasaccharide), fondaparinux sodium (pentasaccharide), and SSR126517.

Indirect Xₐ/IIₐ inhibitors can include, but are not limited to, enoxaparin sodium, (short-acting hexadecasaccharide), AVE5026, SSR128428 (long-acting hexadecasaccharide), and SSR128429.

Diuretics can include, but are not limited to, chlorthalidone, ethacrynic acid, furosemide, amiloride, chlorothiazide, hydrochlorothiazide, methylchtothiazide, and benzthiazide.

Nitrates can include, but are not limited to, isosorbide-5-mononitrate.

Thromboxane antagonists can include, but are not limited to, seratrodast, picotamide and ramatroban,

Platelet aggregation inhibitors can include, but are not limited to, cilostazol, abciximab, limaprost, eptifibatide, and CT-50547.

Cyclooxygenase inhibitors can include, but are not limited to, meloxicam, rofecoxib and celecoxib.

B-type natriuretic peptides can include, but are not limited to, nesiritide and ularitide.

NV1 FGF modulators can include, but are not limited to, XRP0038.

HT1B/5-HT2A antagonists, can include, but are not limited to, SL65.0472.

Guanylate cyclase inhibitors can include, but are not limited to, ataciguat (HMR1766) and HMR1069.

e-NOS transcription enhancers, can include, but are not limited to, AVE9488 and AVE3085

Anti-atherogenics can include, but are not limited to, AGI-1067.

CPU inhibitors can include, but are not limited to, AZD9684.

Renin inhibitors can include, but are not limited to, aliskirin and VNP489.

Inhibitors of adenosine diphosphate (“ADP”) induced platelet aggregation can include, but are not limited to, clopidogrel, ticlopidine, prasugrel, and AZD6140.

NHE-1 inhibitors can include, but are not limited to, AVE4454 and AVE4890.

Other cardiovascular agents can also be used in methods according to the present invention.

Yet another aspect of the present invention is a method for treating or preventing hypertrophy in a cardiomyocyte cell or proliferation of a cardiac fibroblast, comprising contacting the cell with an antagonist of protease activated receptor 1 (PAR1), thereby treating or preventing hypertrophy in the cardiomyocyte cell. Suitable PAR1 antagonists are described above.

### IV. Diseases and Disorders Suitable for Treatment with PAR1 Antagonists

The methods and compositions disclosed in the invention can be employed to treat or prevent cardiac dysfunctions caused by or associated with various cardiac injuries and cardiomyopathies. These include ischemic cardiomyopathies as well as non-ischemic cardiomyopathies. In some of the methods, subjects to be treated are those who have or are at risk of developing an extrinsic cardiomyopathy (e.g., ischemic cardiomyopathy). For example, the subject may have undergone myocardial injuries such as cardiac ischemia/reperfusion or myocardial infarction. Some other methods are directed to treating or preventing cardiac dysfunctions in subjects who have or are at risk of developing non-ischemic cardiomyopathies such as idiopathic dilated cardiomyopathy, viral cardiomyopathy, postpartum cardiomyopathy, or hypertrophic cardiomyopathy. The cardiac dysfunctions to be treated or prevented in the subjects include any complications or medical conditions that may occur or are likely to develop as a consequence to or a result of the development of the cardiomyopathies. Examples of such cardiac dysfunctions include recurrent infarction (recurrent heart attack), cardiac hypertrophy, cardiac fibrosis, cardiac remodeling, congestive heart failure, cardiac death, shock, irregular heart rhythm (arrhythmia), and infarct extension (extension of the amount of affected heart tissue).

Any of the PAR1 antagonist compounds described herein or otherwise known in the art may be employed in the practice of the present invention. In some methods, PAR1 antagonist compounds which specifically inhibit or suppress one or more of PAR1 signaling activities are used (e.g., a peptide, peptidomimetic, or small molecule PAR1 antagonist or anti-PAR-1 antibody). Some other methods can employ compounds which are capable of down-regulating PAR1 expression or cellular level, e.g., short interfering RNA (siRNA), microRNA (miRNA), and synthetic hairpin RNA (shRNA), anti-sense nucleic acids, or complementary DNA (cDNA). In some preferred embodiments, the therapeutic compositions employ a PAR1 antagonist which is specific for PAR1 but has insignificant or no effect on the signaling activities of one or more of the other PAR receptors (PAR2, PAR3, or PAR4). Suitable PAR1 specific antagonists for practicing the present invention include those known in the art (e.g., RWJ-56110 and SCH-79797 described above, as well as others described above) or that can be identified by screening candidate compounds in accordance with the present disclosure. Other PAR1 antagonists, including peptide antagonists, peptidomimetic antagonists, and small molecule antagonists, as well as anti-PAR1 antibodies, are described above and can be used in methods according to the present invention.

Some methods of the present invention are specifically directed to treating or preventing the development or occurrence of cardiac remodeling, cardiac hypertrophy, or heart failure in certain subjects. These subjects include those who have undergone acute ischemic myocardial injuries such as myocardial infarction or cardiac ischemia/reperfusion. Such subjects often develop or are at higher risk of developing cardiac remodeling, cardiac hypertrophy, or heart failure. Therapeutic compositions containing a PAR1 antagonist as described herein can be employed to treat or prevent such cardiac dysfunctions in these subjects.

Subjects who have just endured and survived such myocardial injury can be identified by any of the classical symptoms of acute myocardial infarction. These include chest pain, shortness of breath, nausea, vomiting, palpitations, sweating, and anxiety or a feeling of impending doom. Some myocardial infarctions are silent, without chest pain or other symptoms. These cases can be diagnosed by medical procedures, e.g., electrocardiograms. Medical diagnosis of myocardial infarction can be made by integrating the history of the presenting illness and physical examination with electrocardiogram findings and cardiac markers (blood tests for heart muscle cell damage). These blood tests can include tests for a number of enzymes whose elevated concentration in blood is characteristic of cardiac muscle damage, including creatine kinase, lactate dehydrogenase, and glycogen phosphorylase. In certain cases, particular isozymes of these enzymes are specific for cardiac muscle and elevated concentrations of these isozymes are particularly characteristic of cardiac muscle damage. A coronary angiogram allows visualization of narrowings or obstructions on the heart vessels, and therapeutic measures can follow immediately. A chest radiograph and routine blood tests can indicate complications or precipitating causes and are often performed on admittance to an emergency department. New regional wall motion abnormalities on an echocardiogram are also suggestive of a myocardial infarction and are sometimes performed in equivocal cases. Technetium and thallium can be used in nuclear medicine to visualize areas of reduced blood flow and tissue viability, respectively.

Subjects suspected to be suffering from acute myocardial injury usually need to first get emergency care. These include oxygen, aspirin, glyceryl trinitrate and pain relief. The patient will receive a number of diagnostic tests, such as an electrocardiogram (ECG, EKG), a chest X-ray and blood tests to detect elevated creatine kinase or troponin levels (these are chemical markers released by damaged tissues, especially the myocardium). Further treatment may include either medications to break down blood clots that block the blood flow to the heart, or mechanically restoring the flow by dilatation or bypass surgery of the blocked coronary artery. Coronary care unit admission allows rapid and safe treatment of complications such as abnormal heart rhythms.

Cardiac remodeling is the compensatory or pathological response following myocardial injury. It is viewed as a key determinant of the clinical outcome in heart disorders and a major aspect of the pathology typically seen in the failing heart. The proliferation of interstitial fibroblasts and increased deposition of extracellular matrix components results in myocardial stiffness and diastolic dysfunction, which ultimately leads to heart failure. Subjects that have or are at risk of developing cardiac remodeling typically have or can develop alterations in size, shape and function of the heart (e.g., the left ventricle) in response to changes in hemodynamic loading conditions, neurohormonal activation, or induction of local mediators that alter the structural characteristics of the myocardium. Pathologic remodeling occurs in three major patterns: (a) concentric remodeling when pressure overload causes growth in cardiomyocyte thickness; (b) eccentric remodeling resulting from a volume load that produces cardiomyocyte lengthening; and (c) post-infarction remodeling. Post-infarction remodeling involves a combined pressure and volume load on the non-infarcted area as well as interactions with the cellular and matrix components of the cardiac scar.

Cardiac hypertrophy is an adaptive response of the heart to virtually all forms of cardiac disease, including those arising from hypertension, mechanical load, myocardial infarction, cardiac arrythmias, endocrine disorders, and genetic mutations in cardiac contractile protein genes. While the hypertrophic response is initially a compensatory mechanism that augments cardiac output, sustained hypertrophy can lead to dilated cardiomyopathy, heart failure, and sudden death.

Therapeutic formulations containing PAR1 antagonists described herein are useful to treat or prevent development of the above described symptoms of cardiac hypertrophy and cardiac remodeling. Some of the methods of the invention are specifically directed to treating or preventing hypertrophy of cardiomyocytes and/or proliferation of cardiac fibroblasts (cardiac fibrosis). These methods involve contacting cardiomyocytes and/or cardiac fibroblasts with a PAR1 antagonist compound capable of inhibiting signaling activities mediated by PAR1 or with a compound capable of down-regulating PAR1 expression or cellular level in the cells. In some of these methods, the cardiomyocytes or cardiac fibroblasts are present in vivo in a subject which has undergone acute myocardial injury such as myocardial infarction or cardiac ischemia/reperfusion.

To treat or prevent cardiac remodeling and hypertrophy, the PAR1 antagonist based therapeutic compositions described herein can also be employed in conjunction with other drugs or treatment regimens useful for treating cardiac dysfunctions (e.g., cardiac hypertrophy and remodeling). Many of the therapeutic regimens currently used in the art for improving blood flow and treating heart failure and hypertrophy can be readily employed. For example, aspirin and nitroglycerin are essential for improving blood flow. Nitroglycerin does so by widening the blocked artery, while aspirin does so by thinning the blood and preventing the formation of blood clots. Other blood thinners that can be used to improve blood flow in the blocked artery include Streptokinase (SK), Tissue Plasminogen Activator (TPA), Anisoylated plasminogens streptokinase activator complex, or Heparin. Additional medications needed during acute treatment of a heart attack include β-adrenergic blocking agents (Bristow, Cardiology 92:3-6 (1999)) and angiotensin-converting enzyme (ACE) inhibitors (Eichhorn & Bristow, Circulation 94:2285-2296 (1996)). Other pharmaceutical agents known in the art for treating cardiac hypertrophy include, e.g., angiotensin II receptor antagonists (U.S. Pat. No. 5,604,251 to Heitsch et al.) and neuropeptide Y antagonists (PCT Publication No. WO 98/33791 by Bruce et al.).

In some other methods, PAR1 antagonists are employed to aid in therapeutic or prophylactic treatment of heart failure in subjects who have or are at risk of developing any of the extrinsic cardiomyopathies or intrinsic cardiomyopathies described herein. Heart failure (HF), also called congestive cardiac failure (CCF), is a condition that can result from any structural or functional cardiac disorder that impairs the ability of the heart to fill with or pump a sufficient amount of blood throughout the body. Ischemic heart disease and myocardial infarction are some of the major causes of heart failure. Non-ischemic cardiac injuries such as intrinsic cardiomyopathies can also lead to heart failure. Pharmaceutical composition containing a PAR1 antagonist compound can be administered to the subjects alone or in combination with other known methods or procedures of treating or preventing heart failure. For example, the PAR1 antagonist compounds can be used together with non-pharmacological measures of treating or preventing heart failure such as weight reduction and sodium restriction. They can also be combined with known pharmacological management of heart failure, e.g., drugs such as diuretic agents, vasodilator agents, positive inotropes, ACE inhibitors, beta blockers, and aldosterone antagonists (e.g. spironolactone).

As noted above, there are several types of drugs which have been proven useful in the treatment of heart failure. For example, diuretics help reduce the amount of fluid in the body and are useful for patients with fluid retention and hypertension. Digitalis can be used to increase the force of the heart's contractions, helping to improve circulation. ACE inhibitors can improve survival among heart failure patients and may slow, or perhaps even prevent, the loss of heart pumping activity. Subjects who cannot take ACE inhibitors may get a nitrate and/or a drug called hydralazine, each of which helps relax tension in blood vessels to improve blood flow. Any of these known therapeutic agents for treating cardiac dysfunctions can be employed in combination with the PAR1 antagonists in the practice of the present invention.

The PAR1 antagonist based therapy can also be used in combination with surgical procedures for treating or preventing cardiac dysfunction such as heart failure. For example, a procedure for severe heart failure available called cardiomyoplasty can be used in combination with the present invention (Dumcius et al., Medicina 39:815-822 (2003)). This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

PAR1 antagonist based therapy can also be employed in conjunction with the implantation of cardiac devices in some subjects with cardiac disorders. For example, subjects receiving cardiac resynchronization therapy (CRT, such as biventricular pacing) can be administered a therapeutic composition containing a PAR1 antagonist. In some subjects with advanced heart failure, biventricular pacing (a pacemaker that senses and initiates heartbeats in the right and left ventricle) improves survival, reduces symptoms and increases exercise capacity or tolerance. For people with heart block or some bradycardias (slow heart rates), this pacemaker will also serve to maintain an adequate heart rate. A PAR1 antagonist based regimen could provide additional prophylactic benefits to subjects receiving such cardiac devices. Similarly, subjects who have implantable cardioverter defibrillators (ICD) can also benefit from the concurrent treatment with acute PAR1 antagonist based therapy. ICDs are suggested for people at risk for life-threatening ventricular arrhythmias or sudden cardiac death. The ICD constantly monitors the heart rhythm. When it detects a very fast, abnormal heart rhythm, it delivers energy (shock) to the heart muscle to cause the heart to beat in a normal rhythm again. A cardiac resynchronization therapy and a cardioverter defibrillator may be combined in a single device labeled CRT-D. A PAR1 antagonist based therapeutic composition can be administered to a subject having a CRT, a ICD, a CRT-D or other implantable cardiac devices in accordance with the present application.

### V. Pharmaceutical Compositions and Methods of Administration

The PAR1 antagonists and the other therapeutic agents described above can be administered directly to subjects in need of treatment. However, therapeutic agents are preferably administered to the subjects in pharmaceutical compositions which comprise the PAR1 antagonist and/or other active agents in a therapeutically effective dose along with a pharmaceutically acceptable carrier, diluent or excipient in unit dosage form. Pharmaceutically acceptable carriers are agents which are not biologically or otherwise undesirable, i.e. the agents can be administered to a subject along with a PAR1 antagonist without causing any undesirable biological effects or interacting in a deleterious manner with any of the components of the pharmaceutical composition in which it is contained. The compositions can additionally contain other therapeutic agents that are suitable for treating or preventing cardiac dysfunctions described above, such as, but not limited to, an antagonist of PAR2, a monoclonal antibody specifically binding to TF that specifically blocks signaling activity of TF without substantially interfering with coagulation activity of TF, or an additional therapeutic agent. Pharmaceutically acceptable carriers enhance or stabilize the composition, or can facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier should be suitable for various routes of administration described herein.

Therefore, pharmaceutical compositions according to the present invention for the treatment or prevention of a cardiovascular dysfunction in a subject comprise:

(1) a therapeutically effective dose of a PAR1 antagonist; and

(2) a pharmaceutically acceptable carrier, diluent or excipient in unit dosage form.

A pharmaceutical composition containing a PAR1 antagonist described herein and/or other therapeutic agents can be administered by a variety of methods known in the art. The routes and/or modes of administration vary depending upon the desired results. Depending on the route of administration, the active therapeutic agent may be coated in a material to protect the compound from the action of acids and other compounds that may inactivate the agent. Conventional pharmaceutical practice can be employed to provide suitable formulations or compositions for the administration of such antagonists to subjects. Any appropriate route of administration can be employed, for example, but not limited to, intravenous, parenteral, intraperitoneal, intravenous, transcutaneous, subcutaneous, intramuscular, intracranial, intraorbital, intraventricular, intracapsular, intraspinal, topical, intranasal, epidural, pulmonary, or oral administration. Depending on the specific cardiac dysfunction and other conditions of the subject to be treated, either systemic or localized delivery of the therapeutic agents can be used in the course of treatment.

In some embodiments, local administration of PAR1 antagonists is desired in order to achieve the intended therapeutic effect. Many methods of localized delivery of therapeutic agents or formulations can be used in the practice of the invention. For example, local administration of a PAR1 antagonist to the desired cardiac muscle in a subject can be accomplished by a percutaneous route, by therapeutic cardiac catheterization, by intrapericardial injection or infusion, or by direct intracardiac muscle injection. Suitable methods also include any other routes which allow the therapeutic agent to be applied locally to the heart. For example, the therapeutic agent may be applied from the bloodstream, by being placed directly in the heart through the coronary arteries or veins onto the heart surface, or through the ventricular or atrial walls and onto the heart surface. The therapeutic agent may also be applied through direct application during extensive surgical field exposure, or through direct application during minimally invasive exposure, e.g., through a pericardial window or heart port.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes, Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or can be oily solutions for administration in the form of nasal drops, or as a gel.

The PAR1 antagonists for use in the methods of the invention are typically administered to a subject in an amount that is sufficient to achieve the desired therapeutic effect (e.g., eliminating or ameliorating symptoms associated with cardiac dysfunctions) in a subject in need thereof. Typically, a therapeutically effective dose or efficacious dose of the PAR1 antagonist is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, or the ester, salt or amide thereof, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated.

The PAR1 antagonist compound and other therapeutic regimens described above are usually administered to the subjects on multiple occasions. Intervals between single dosages can be weekly, monthly or yearly. Intervals can also be irregular as indicated by measuring blood levels of the PAR1 antagonist compound and the other therapeutic agents used in the subject. In some methods, dosage is adjusted to achieve a plasma compound concentration of 1-1000 μg/ml and in some methods 25-300 μg/ml. Alternatively, the therapeutic agents can be administered as a sustained release formulation, in which case less frequent administration is required. Dosage and frequency vary depending on the half-life in the subject of the PAR1 antagonist compound and the other drugs included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

For the purposes of the present application, treatment can be monitored by observing one or more of the improved symptoms of heart failure or cardiac hypertrophy. The symptoms of heart failure or cardiac hypertrophy include, but are not limited to, such symptoms as reduced exercise capacity, reduced blood ejection volume, increased left ventricular end diastolic pressure, increased pulmonary capillary wedge pressure, reduced cardiac output, decreased cardiac index, increased pulmonary artery pressures, increased left ventricular end systolic and diastolic dimensions, and increased left ventricular or right ventricular wall stress, wall tension or wall thickness. Therefore, administration of a PAR1 antagonist according to the present invention would be expected to result in changes such as, but not limited to, increased exercise capacity, increased blood ejection volume, decreased left ventricular end diastolic pressure, decreased pulmonary capillary wedge pressure, increased cardiac output, increased cardiac index, decreased pulmonary artery pressures, decreased left ventricular end systolic and diastolic dimensions, and decreased left ventricular or right ventricular wall stress, wall tension or wall thickness.

The preparations formulated for oral administration may be in the form of tablets, dragees, capsules, or solutions. The pharmaceutical compositions contemplated by the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active modulators in water-soluble form. Additionally, suspensions of the PAR1 antagonists can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility of the PAR1 antagonists to allow for the preparation of highly concentrated solutions. Pharmaceutical preparations for oral use can be obtained by combining the active modulators with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different doses of PAR1 antagonists.

Pharmaceutical preparations which can be used orally include push-fit capsules made of gelatins as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the PAR1 antagonists may be dissolved or suspended in suitable liquids, such as fatty oils, liquid paraffin, or liquid polyethylene glycols. In addition, stabilizers may be added.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a PAR1 antagonist or compound down-regulating the expression or cellular level of PAR1 by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the active PAR1 antagonist or compound down-regulating the expression or cellular level of PAR1, such as aqueous solubility, partition coefficient, molecular size, stability of the active PAR1 antagonist or compound down-regulating the expression or cellular level of PAR1, and binding of the active PAR1 antagonist or compound down-regulating the expression or cellular level of PAR1 to proteins and other biological macromolecules. This process of preparation also takes into account biological factors, such as absorption, distribution, metabolism, duration of action, the possible existence of side effects, and margin of safety, for the active PAR1 antagonist or compound down-regulating the expression or cellular level of PAR1. Accordingly, one of ordinary skill in the art could modify the formulations in order to incorporate an active active PAR1 antagonist or compound down-regulating the expression or cellular level of PAR1 into a formulation having the desirable properties described above for a particular application.

In another alternative, the pharmaceutical composition can further comprise a therapeutically effective amount of a second therapeutic agent for cardiac dysfunction. The second therapeutic agent can be, for example, an antagonist of protease activated receptor 2 (PAR2), a monoclonal antibody specifically binding to TF that specifically blocks signaling activity of TF without substantially interfering with coagulation activity of TF, or an additional therapeutic agent selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACE/NEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIa inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibitors, renin inhibitors, inhibitors of adenosine diphosphate (“ADP”)-induced platelet aggregation, and NHE-1 inhibitors.

The invention is illustrated by the following examples. These examples are for illustrative purposes only, and are not intended to limit the invention.

### EXAMPLES

### Example 1

### Materials and Methods

Mice. PAR1^{+/-} mice were backcrossed 11 generations onto a C57BI/6J background and bred to generate PAR1^{+/+} and PAR1^{-/-} littermate mice. αMHC-Cre mice were a generous gift from Dr. E. Abel (University of Utah School of Medicine). This study was performed in accordance with the guidelines of the Animal Care and Use Committees of The Scripps Research Institute, La Jolla, Calif., the University of Washington, Seattle, Wash., and the University of Rochester, Rochester, N.Y. and complies with NIH guidelines.

Generation of αMHC-PAR1 mice. A 1.3 kbp DNA fragment containing the coding sequence of mouse PAR1 was cloned into a vector containing the cardiomyocyte-specific αMHC promoter (kindly provided by Dr. F. Naya). This promoter is a promoter that drives gene expression in cardiomyocytes. The background of these mice is C57BL/6. Next, an 8.5 kbp Not1 fragment, which contained the αMHC promoter, the coding sequence for mouse PAR1 and the human growth hormone polyA sequence, was purified and injected into the pronucleus of fertilized mouse embryos (C57BI/6J genetic background) by The Scripps Transgenic Core Facility. Transgenic mice were identified by PCR using primers for the human growth hormone polyA sequence.

Cardiac I/R injury models. For the short-term I/R model (30 minutes of ischemia and 2 hours of reperfusion), the surgical protocol and infarct size determination were performed as described in Palazzo et al. (Am. J. Physiol. 275:H2300-H2307 (1998)) with some modifications. Briefly, intraperitoneal injection of pentobarbital (100 mg/kg) (Abbott Laboratories, Abbott Park, Ill.) was used for anesthesia. Mice were orally intubated to provide artificial ventilation (0.3 mL tidal volume, 120 breaths/minute). The left anterior descending coronary artery was occluded with a 7-0 silk suture (U.S. Surgical Corp., Norwalk, Conn.) passed through PE tubing (U.S. Surgical Corp.) to make a Rumel snare. After 30 minutes of ischemia, the snare was released and the heart was reperfused for 2 hours. Finally, the artery was reoccluded and 4% Evans Blue dye was injected into the aortic root to delineate the area-at-risk (AAR) from not at-risk myocardium (blue). Hearts were then explanted, rinsed in 0.9% normal saline, and placed in 1% agarose gel (UltraPure agarose, Life Technologies, Gaithersburg, Md.) in PBS (pH 7.4). Hearts were sectioned parallel to the AV groove in ˜1 mm sections. Viable and necrotic areas of the AAR were identified by incubating the hearts in 1% 2,3,5-triphenyltetrazolium chloride (Sigma-Aldrich, St. Louis, Mo.) for ten minutes at 37° C., followed by 10% neutral buffered formaldehyde for 24 hours. Each section was weighed and photographed. The LV, AAR and infarct areas were traced and calculated by computer planimetry (Image J, version 1.21). Infarct volumes were calculated as: [(A1×W1)+(A2×W2)+(A3×W3)+(A4×W4)+(A5×W5)], where A is the area of infarct for the slice denoted by the subscript and W is the weight of the respective section.

For the long-term model (45 minutes of ischemia and 2 weeks of reperfusion), mice were anesthetized with 2% halothane and 40% oxygen, and maintained with 0.5% halothane and 40% oxygen for the duration of the surgery. Mice were orally intubated to provide artificial ventilation (0.3 mL tidal volume, 120 breaths/minute). Following left thoracotomy at the fourth intercostal space, the left anterior descending coronary artery was ligated with an 8-0 nylon surgical suture 2.0 mm distal from the tip of the left atrium, and occluded for 45 minutes. Ischemia was validated via ECG recordings. Following occlusion, the suture was released, the chest sutured closed in 2 stages and the mice allowed to recover. Surgery on wild type (WT) and PAR1^{-/-} mice was performed in a blinded fashion.

Echocardiography. Echocardiography was performed on conscious mice using an Acuson Sequoia C236 echocardiography machine equipped with a 15 MHz linear probe (Siemens Medical Solutions, Malvern, Pa.). LV function was measured by M-mode echocardiography in the short axis view at the mid-ventricular level. The percentage of fractional shortening was assessed by measuring the end diastolic and end systolic diameter [(end diastolic diameter-end systolic diameter)/end diastolic diameter×100(%)]. The mean velocity of circumferential fiber shortening (mVcf) was calculated by dividing the fractional shortening by the ejection time multiplied by the square root of the R—R interval. Echocardiography on WT, PAR1^{-/-} and αMHC-PAR1 mice was performed in a blinded fashion.

Human heart samples. For analysis of PAR1 mRNA expression, tissue was obtained from the LV free wall (toward the apex) of 9 male patients in end-stage heart failure at the time of left ventricular assist device (LVAD) placement or transplant. Non-failing tissue was obtained from the LV free wall (toward the apex) of 5 male non-failing organ donor hearts rejected for transplant for physical incompatibilities. LV tissue obtained from surgery was immediately frozen in liquid nitrogen and stored at −80° C. All surgical procedures and tissue harvesting were performed in concordance with NIH and University of Rochester Institutional Review Board guidelines. Only viable, non-ischemic cardiac tissue was used. For analysis of PAR1 protein expression, heart tissue was collected from ventricular areas of hearts obtained from autopsies as described in Luther et al. (J. Pathol. 192:121-130 (2000)). The degree of hypertrophy was graded by determination of thickness of the LV (hypertrophy of cardiac muscle: <14 mm, no hypertrophy; 14-18 mm, moderate; >18 mm, high).

Northern blotting. Levels of ANF, BNP, PAR1 and GAPDH were determined by northern blotting using standard protocols.

Western blot analysis and immunohistochemistry. Protein extracts were generated from mouse hearts as described (22). For the preparation of protein extracts from human hearts, thin sections of frozen tissue samples were homogenized in ice-cold extraction buffer (20 mM Tris/HCl, 0.125 M NaCl, 1% Triton-X-100, pH 8.5). For extraction, homogenates were gently agitated at 4° C. for 6 hours followed by centrifugation at 14,000×g for 15 minutes. The supernatants were aliquoted and stored frozen at −80° C. until used. Protein (20-25 μg) was subjected to SDS-PAGE and transferred to either nitrocellulose or polyvinylidene difluoride (PVDF) membranes. Human PAR1 was detected using the mouse anti-human PAR1 mAb ATAP 2 (Santa Cruz Biotechnology Inc., Santa Cruz, Calif.). Mouse PAR1 was detected using the goat anti-human PAR1 antibody C-18 (Santa Cruz Biotechnology), PAR1 expression in frozen mouse heart sections was detected using the H111 anti-PAR1 antibody (Santa Cruz Biotechnology) and the Vectastain Elite ABC system (Vector Laboratories, Burlingame, Calif.).

Real Time PCR. Levels of PAR1 mRNA in human hearts were determined by real time PCR using standard Taqman® technology (Applied Biosystems, Foster City, Calif.). Primers and probe sequences for human PAR1 mRNA have been described in Hamilton et al. (Circ. Res. 89:92-98 (2001)).

Determination of the lumenal area of LV and the thickness of the LV wall. Cross-sections of hearts at the level of papillary muscles were obtained and the lumenal area of LV and the thickness of LV wall were measured using an NIH Scion image program.

Measurement of apoptosis. Apoptotic cell death was analyzed using TACS Apoptotic DNA Laddering Kits (R&D Systems, Minneapolis, Minn.) according to manufacturer's instruction. In addition, apoptotic cell death was analyzed by TUNEL-based CardioTACS in situ Apoptosis Detection Kit (R&D Systems).

Statistical analysis. Comparisons between the different groups were performed using a Student's t-test for unpaired data. Statistical differences were considered significant for P values <0.05. Data are shown as mean±SEM.

### Example 2

### PAR1 Deficiency Does Not Affect Infarct Size After I/R Injury

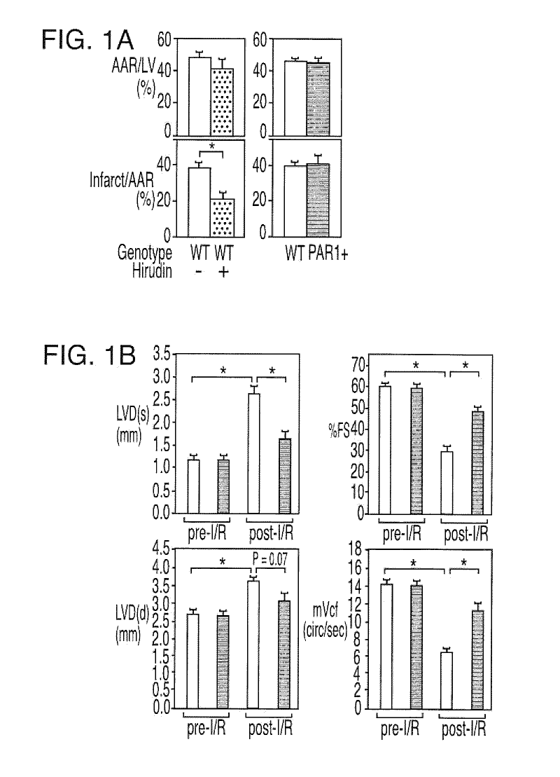

Based on our previous studies showing that inhibition of either TF or thrombin reduced infarct size (Erlich et al., Am. J. Pathol. 157:1849-1862 (2000)), we hypothesized that PAR1 deficiency would reduce infarct size. We used a mouse model of short-term cardiac I/R injury that consists of 30 minutes of ischemia and 2 hours of reperfusion. Surprisingly, we found no significant difference in the infarct size between PAR1^{-/-} mice and WT littermates (FIG. 1A). In contrast, inhibition of thrombin with hirudin decreased infarct size (FIG. 1A). Neither hirudin treatment nor PAR1 deficiency affected the size of the area at-risk. Furthermore, PAR1 deficiency did not affect the induction of various inflammatory mediators (IL-1β, IL-6, MIP-2 and MCP-1) in the injured hearts (data not shown). These results indicate that PAR1 does not contribute to infarct size or inflammation after I/R injury.

### Example 3

### PAR1^{-/-} Mice Have Reduced Cardiac Remodeling After I/R Injury

The majority of cardiac remodeling after myocardial infarction is due to hypertrophy of cardiomyocytes and proliferation of cardiac fibroblasts. Despite the fact that PAR1 did not contribute to infarct size, we hypothesized that PAR1 signaling plays a role in cardiac remodeling after myocardial infarction by inducing hypertrophy of cardiomyocytes and cell proliferation of cardiac fibroblasts. We used a long-term mouse model of cardiac I/R injury consisting of 45 minutes of ischemia and 2 weeks of reperfusion to induce cardiac remodeling. Echocardiography showed that after cardiac I/R injury the hearts of WT mice exhibited dilation of the LV, significant impairment of LV function and thinning of the posterior LV wall at systole (FIG. 1B). In contrast, PAR1^{-/-} mice exhibited strikingly less dilation of the LV and reduced impairment of LV function (FIG. 1B). Similar results were observed in an independent experiment with PAR1^{-/-} mice (n=5) and WT littermates (n=5) (data not shown). These results indicate that PAR1 plays a role in cardiac remodeling after myocardial infarction.

### Example 4

### PAR1^{-/-} Mice have Reduced Cardiac Remodeling after I/R Injury

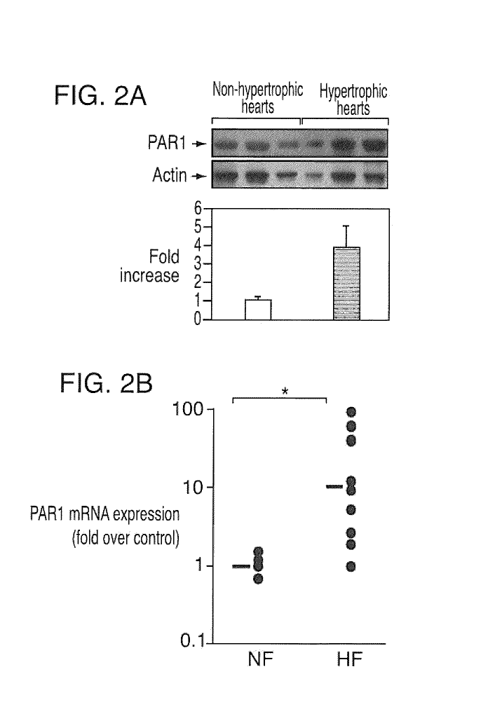

We speculated that if PAR1 contributes to cardiac remodeling, then its expression may be increased in hypertrophic and failing hearts. We found that PAR1 protein expression was upregulated in hearts of patients with cardiac hypertrophy (FIG. 2A). In addition, PAR1 mRNA levels were significantly higher in patients with failing hearts (FIG. 2B). These results support the notion that PAR1 contributes to cardiac remodeling.

### Example 5

### Generation of Mice Overexpressing PAR1 in Cardiomyocytes

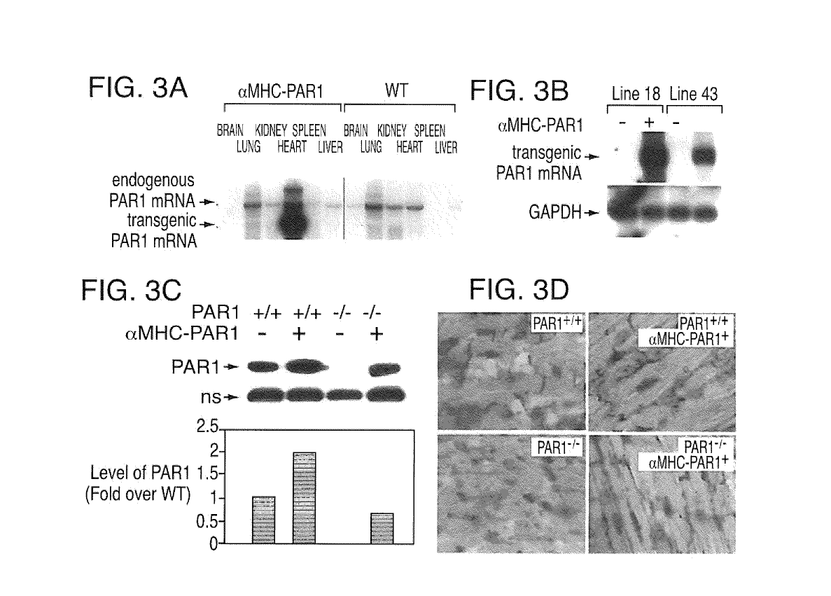

Upon the observations that PAR1 expression is upregulated in human and mouse failing hearts (FIG. 2), we then determined the effect of overexpressing PAR1 on cardiomyocytes on the development and function of the heart. Two transgenic mouse lines (αMHC-PAR1 line 18 and αMHC-PAR1 line 43) were made that exhibited different levels of cardiac-specific PAR1 mRNA expression (FIGS. 3A and B). Importantly, despite the high level of overexpression of PAR1 mRNA from the αMHC-PAR1 transgene, PAR1 protein expression was only increased approximately 2 fold in hearts of αMHC-PAR1 mice line 18 compared with WT littermates (FIG. 3C). This level of overexpression is similar to the increased levels observed in hypertrophic human hearts (FIG. 2A).

To analyze PAR1 expression exclusively from the transgene, we crossed line 18 with PAR1^{-/-} mice to produce αMHC-PAR1/PAR1^{-/-} mice. PAR1 protein was detected in the hearts of αMHC-PAR1/PAR1^{-/-} mice, but not in PAR1^{-/-} mice (FIGS. 3C and D). Immunohistochemical studies demonstrated that PAR1 expressed from the transgene was localized to the plasma membrane of the cardiomyocytes in a similar manner to PAR1 expressed by the endogenous gene (FIG. 3D). These data indicate that we generated transgenic mice with increased levels of PAR1 on cardiomyocytes similar to those observed in human heart hypertrophy.

### Example 6

### PAR1 Overexpression in Cardiomyocytes Induces Eccentric Hypertrophy and Dilated Cardiomyopathy

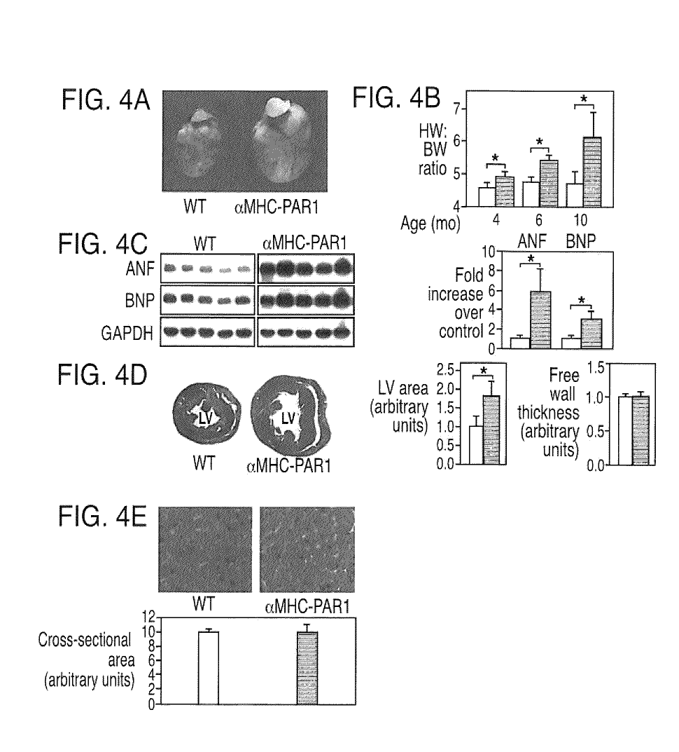

We analyzed the hearts of αMHC-PAR1 mice and WT littermates to determine if PAR1 overexpression in cardiomyocytes affected cardiac development or cardiac function in adult mice. The hearts of young αMHC-PAR1 mice (<3 months of age) were indistinguishable from those of WT littermate controls, indicating that overexpression of PAR1 did not affect development of the heart (data not shown). In contrast, hearts of older αMHC-PAR1 line 18 mice (10 months of age) were larger than the hearts of WT littermates (FIG. 4A). In addition, we observed an age-dependent increase in the heart weight/body weight (HW:BW) ratio (FIG. 4B). The weight of other organs (lung, kidney and liver) compared to body weight was unchanged in αMHC-PAR1 mice (data not shown). At 12 months of age, αMHC-PAR1 line 43 mice also exhibited an increase in HW:BW ratio (4.97±0.36 versus 4.54±0.26 in αMHC-PAR1 mice and WT littermate controls, respectively; P<0.02; n=15 for each group). However, this increase was smaller than the hypertrophy observed with line 18, indicating a positive correlation between the level of transgene expression and the degree of hypertrophy. All subsequent studies were performed using αMHC-PAR1 line 18 mice.

Next, we analyzed the expression of two fetal genes that are induced during hypertrophy. αMHC-PAR1 mice exhibited a significant increase in the expression of ANF and BNP compared with WT littermates at 4 and 6 months (FIG. 4C and data not shown). Histological analysis of the hearts showed that the lumenal area of the LV was significantly increased in αMHC-PAR1 mice (FIG. 4D). However, there was no difference in the thickness of the free LV wall (FIG. 4D). In addition, there was no difference in the cross-sectional area of cardiomyocytes in the hearts of αMHC-PAR1 mice and WT littermates (FIG. 4E). These data suggest that the increase in heart size of αMHC-PAR1 mice is mainly due to elongation of cardiomyocytes.

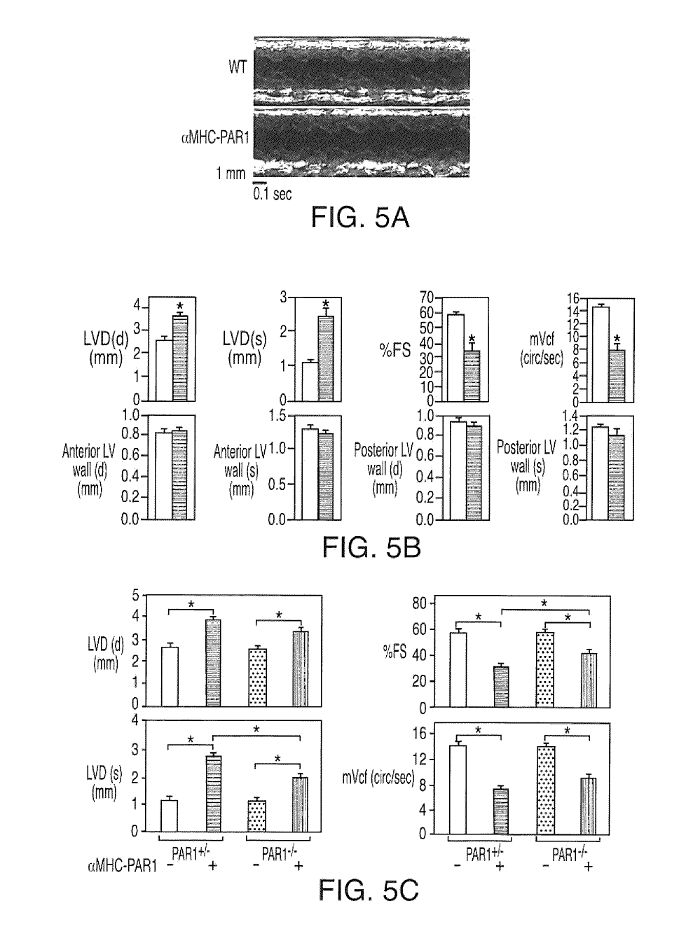

Finally, we used transthoracic echocardiography to measure the diameter of the LV, thickness of the ventricular walls and LV function. FIG. 5A shows representative M-mode echocardiographic images of an αMHC-PAR1 and a WT littermate. Consistent with our histological analysis, we found that the diameter of the LV was significantly increased in αMHC-PAR1 mice compared with WT littermates at 10 months of age (FIG. 5B). In contrast, there was no difference in the thickness of the anterior and posterior LV wall between the two groups of mice (FIG. 5B). As expected, LV function was significantly reduced in αMHC-PAR1 mice compared with WT littermates (FIG. 5B). Taken together, these findings are consistent with our histological analysis and indicate that overexpression of PAR1 on cardiomyocytes induces eccentric cardiac hypertrophy (increased LV dimension and normal LV wall thickness).

To analyze the relative contribution of PAR1 expressed from the endogenous gene and the transgene to hypertrophy, we analyzed hypertrophy in the absence of endogenous PAR1 expression. LV function and the size of the LV were not different between PAR1^{-/-} and PAR1^{+/-} mice (FIG. 5C), indicating that the absence of PAR1 does not affect cardiac development or LV function. Importantly, αMHC-PAR1^{-/-} mice exhibited dilation of the LV and impaired LV function that was less severe than αMHC-PAR1/PAR1^{+/-} mice (FIG. 5C). These results demonstrate a dose-response of PAR1 overexpression, and suggest that PAR1 from the transgene combines with PAR1 expressed from the endogenous gene to induce dilated cardiomyopathy.

### Example 7

### Cardiomyocyte-Specific Deletion of the TF Gene Reduces Hypertrophy in αMHC-PAR1 Mice

Based on our studies showing that inhibition of TF or thrombin reduces cardiac I/R injury (J. H. Erlich et al., “Inhibition of the Tissue Factor-Thrombin Pathway Limits Infarct Size After Myocardial Ischemia/Reperfusion Injury by Reducing Inflammation,” Am. J. Pathol. 157: 1849-1862 (2000)), we hypothesized that coagulation proteases activate PAR1 signaling in αMHC-PAR1 mice and induce eccentric hypertrophy. To test this hypothesis, we determined the effect of deleting the TF gene in cardiomyocytes on the cardiac hypertrophy of αMHC-PAR1 mice. Mice with a cardiomyocyte-specific deletion of the TF gene were generated by crossing TF^{flox/flox} mice with mice expressing the Cre recombinase under the control of the αMHC promoter. TF mRNA expression in the hearts of TF^{flox/flox}/αMHC-Cre mice was reduced by 66% compared to TF^{flox/flox} mice (n=6 for both groups; P<0.0005). In contrast to our previous study showed that reducing TF levels to below 1% of WT levels led to hemorrhage and fibrosis in the heart (Pawlinski et al., Proc. Natl. Acad. Sci. U.S.A. 99: 15333-15338 (2002)), the reduction of TF expression in TF^{flox/flox}/αMHC-Cre mice was not associated with an impairment of heart hemostasis. Importantly, TF mRNA expression was reduced by 98% in cardiomyocytes isolated from TF^{flox/flox}/αMHC-Cre neonates (mean of 3 independent preparations). In a recent study, we also found that cardiac fibroblasts express TF, which explains the difference in the level of deletion of TF in the hearts and isolated cardiomyocytes.

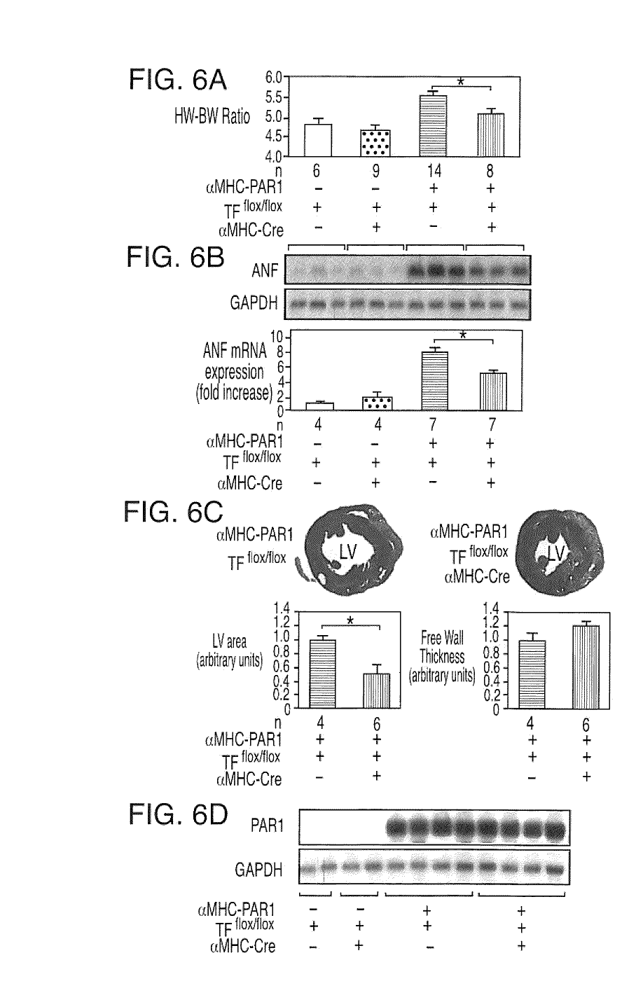

αMHC-PAR1 mice with cardiomyocyte-specific deletion of TF gene (αMHC-PAR1/TF^{flox/flox}/αMHC-Cre) were generated in two steps from crosses between αMHC-PAR1 and TF^{flox/flox}/αMHC-Cre mice. These mice were then crossed with TF^{flox/flox} mice to generate four different groups of mice to analyze the effect of deletion of the TF gene on PAR1-dependent hypertrophy. We found that deletion of the TF gene significantly reduced the HW:BW ratio and ANF mRNA expression in the hearts of αMHC-PAR1/TF^{flox/flox}/αMHC-Cre mice compared with αMHC-PAR1/TF^{flox/flox} littermate controls (FIGS. 6A and B). In addition, the lumenal area of the LV was significantly reduced in αMHC-PAR1/TF^{flox/flox}/αMHC-Cre mice without any effect on the thickness of the LV free wall (FIG. 6C). These results indicate that PAR1 overexpressed in αMHC-PAR1 mice is activated, in part, by TF-dependent coagulation proteases.

To exclude the possibility that the presence of the αMHC-Cre transgene reduced PAR1 expression from the αMHC-PAR1 transgene by competing for transcription factors, we analyzed PAR1 mRNA expression in αMHC-PAR1/TF^{flox/flox}/αMHC-Cre mice and αMHC-PAR1/TF^{flox/flox} littermate controls. Similar levels of PAR1 mRNA expression were observed in these two groups of mice (FIG. 6D), indicating that the reduced hypertrophy observed in the αMHC-PAR1/TF^{flox/flox}/αMHC-Cre mice was not due to reduced PAR1 expression.

### Example 8

### Effect of Knockout of PAR2 Function on Infarct Size

The effect of the knockout of PAR2 function on infarct size in mice was determined. Knockout mice for PAR2 were generated as described in B. P. Damiano et al., “Cardiovascular Responses Mediated by Protease-Activated Receptor-2 (PAR-2) and Thrombin Receptor (PAR-1) Are Distinguished in Mice Deficient in PAR-2 or PAR-1,”J. Pharmacol. Exp. Ther. 288: 671-678 (1999). These PAR2^{-/-} mice were subjected to cardiac/IR injury as described above in Example 1, using 30 minutes of ischemia and 2 hours of reperfusion.

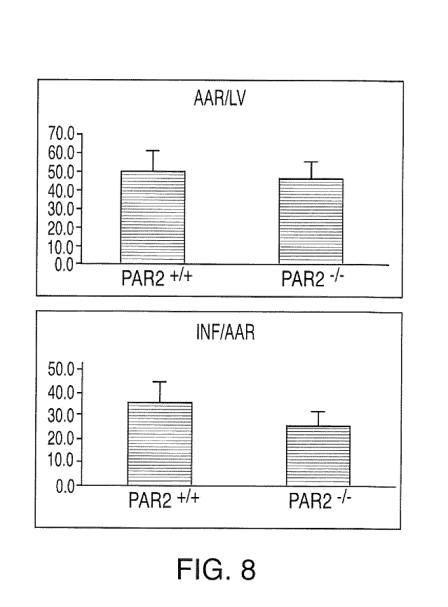

The results are shown in FIG. 8. In FIG. 8, the top panel shows ratio of the area of risk (AAR) versus the left ventricle (LV). Error bars show the standard deviation (SD). The left bar shows the results for wild-type (PAR2^{+/+}) mice, while the right bar shows the results for knockout (PAR2^{-/-}) mice. The bottom panel shows the ratio of the area of the infarct (INF) versus the area of risk. Again, the bar shows the results for wild-type (PAR2^{+/+}) mice, while the right bar shows the results for knockout (PAR2^{-/-}) mice.

The results of FIG. 8 shows that inhibition of PAR2 function reduces the infarct size, as PAR2 knockout mice have a reduced infarct size compared with wild-type mice.

### ADVANTAGES OF THE INVENTION

The present invention provides a new means of treating a number of diseases and conditions affecting the cardiovascular system, including cardiac hypertrophy, cardiomyopathy, and heart failure, particularly following acute myocardial infarction (heart attack). This new means of treating these diseases and conditions acts through inhibition of the activity of the protease-activated G-protein-coupled receptor PAR1.

The methods and compositions according to the present invention can be used together with other commonly accepted medical and surgical methods of treating such diseases and conditions affecting the cardiovascular system, including administration of drugs, lifestyle changes such as weight reduction and exercise, and surgery. These methods and compositions are well tolerated and do not produce side effects because of the specific effects of inhibiting the activity of PAR1 in cardiomyocytes.

Methods and compositions according to the present invention possess industrial applicability, specifically for the preparation of medicaments to treat cardiovascular diseases and conditions such as, but not limited to, cardiac hypertrophy, cardiomyopathy, and heart failure.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art. Similarly, all GenBank sequences and other sequence information obtainable from publicly accessable databases are herein incorporated by reference as if each was specifically and individually indicated to be incorporated by reference.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

We claim:

1. A method for treating or preventing a cardiac dysfunction in a subject, comprising administering to a subject a therapeutically effective amount of an antagonist of protease activated receptor 1 (PAR1), thereby treating or preventing cardiac dysfunction in the subject, wherein the subject:
(a) has or is at risk of developing a cardiomyopathy; or
(b) has undergone a myocardial injury.

2. The method of claim 1, wherein the cardiac dysfunction is cardiac hypertrophy, cardiac remodeling, or heart failure.

3. The method of claim 1, wherein the subject has developed or is at risk of developing an extrinsic cardiomyopathy.

4. The method of claim 3, wherein the extrinsic cardiomyopathy is ischemic cardiomyopathy.

5. The method of claim 3, wherein the subject has undergone a myocardial injury.

6. The method of claim 5, wherein the myocardial injury is selected from the group consisting of myocardial infarction and cardiac ischemia/reperfusion.

7. The method of claim 1, wherein the subject has developed or is at risk of developing an intrinsic cardiomyopathy.

8. The method of claim 7, wherein the intrinsic cardiomyopathy is selected from the group consisting of idiopathic dilated cardiomyopathy, viral cardiomyopathy, post-partum cardiomyopathy, and hypertrophic cardiomyopathy.

9. The method of claim 1, wherein the PAR1 antagonist is a PAR1-selective antagonist.

10. The method of claim 9, wherein the PAR1 selective antagonist has a binding affinity for PAR1 that is at least 5-fold stronger than the binding affinity of the PAR1 selective antagonist for any of PAR2, PAR3, and PAR4.

11. The method of claim 1, wherein the PAR1 selective antagonist binds to PAR1 with an IC₅₀ that is less than 1 μM.

12. The method of claim 1, wherein the PAR1 selective antagonist binds to all of PAR2, PAR3, and PAR4 with an IC₅₀ that is at least 5 μM.

13. The method of claim 1, wherein the PAR1 antagonist is selected from the group consisting of a peptide, a peptidomimetic, a small molecule organic compound, a pepducin, a polynucleotide, and an antibody.

14. The method of claim 13 wherein the PAR1 antagonist is a peptide of the sequence X₁-F(f)F(Gn)LR-NH₂, where X₁ is selected from the group consisting of (2-thiophene)acetyl, N-acetyl-2-aminobenzoyl, 2-oxo-(2-thiophene)acetyl, (3-thiophene)acetyl, phenylacetyl, (2-thiophene)sulfonyl, (3-fluorophenyl)acetyl, (4-fluorophenyl)acetyl, 3-pyridylacetyl, (2-fluorophenyl)acetyl, (3-indole)acetyl, cyclopentylacetyl, 2-oxo(3-indole)acetyl, 3-indoloyl, (3-chlorophenyl)acetyl, N-acetyl-4-aminobutyryl, 2-thiopheneoyl, 3-thiopheneoyl, 3-furanoyl, 2-indoloyl, 4-chlorobenzoyl, trans-cinnamoyl, 3-phenyl-2-propynoyl, p-fluoro-trans-cinnamoyl, p-chloro-trans-cinnamoyl, p-methyl-trans-cinnamoyl, p-methoxy-trans-cinnamoyl, 4-biphenyloyl, m-chloro-trans-cinnamoyl, 3-phenylpropionyl, phenoxyacetyl, 1-napthtylacetyl, 3-(2-thiophene)-trans-acryloyl, (±)-trans-3-phenylcyclopropanoyl, 3-coumarinyl, 4-phenylbutyryl, p-amino-trans-cinnamoyl, 2-naphthylacetyl, p-hydroxy-trans-cinnamoyl, (thiophenoxy)acetyl, trans-2-trans-4-hexadienoyl, trans-2-octenoyl, α-fluorocinnamoyl, α-methylcinnamoyl, and α-phenylcinnamoyl, F(f) is a substituted phenylalanine residue with a fluoro group at the para position of the phenyl moiety of the phenylalanine, and F(Gn) is a substituted phenylalanine residue with a guanidino group at the para position of the phenyl moiety of the phenylalanine.

15. The method of claim 14 wherein the PAR1 antagonist is a peptide of the sequence trans-cinnamoyl-F(f)-F(Gn)-L-X₂, in which X₂ is selected from the group consisting of Orn-NH₂, Orn(acetyl)-NH₂, Arg-Orn-NH₂, Arg-Orn(N⁵-acetyl)-NH₂, Arg-Arg-NH₂, and Arg-Orn(N⁵-propionyl)-NH₂.

16. The method of claim 14 wherein the PAR1 antagonist is selected from the group consisting of N-[3-methyl-1-S[[2-S-[(methyl)aminocarbonyl]-1-pyrrolidinyl]carbonyl]butyl-D-alanine and N-[3-methyl-S-(1-pyrrolidinylcarbonyl)butyl-D-alanine ethyl ester hydrochloride.

17. The method of claim 14 wherein the PAR1 antagonist is a PAR1 antagonist of Formula (II)

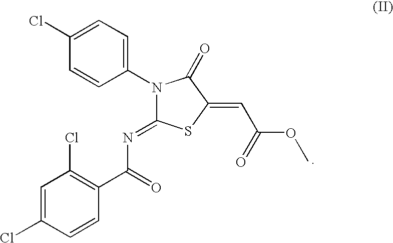

18. The method of claim 14 wherein the PAR1 antagonist is a trisubstituted urea with a structure selected from the group of:
(a) trisubstituted ureas of Formula (III), wherein R can be t-butyl-, NH₂CMe₂-, 2,4-difluorophenyl-, 3-pyridyl, 4-(SO₂NH₂)phenyl- and 4-benzimidazole-;
(b) trisubstituted ureas that are derivatives of Formula (III), wherein the 2-propyl substituent on the nitrogen atom of the central urea moiety of Formula (III) is replaced with a substituent selected from the group consisting of n-propyl, cyclobutyl, i-propyl, (±)2-pentyl, (S)sec-butyl, or (R)sec-butyl; and
(c) trisubstituted ureas of Formula (IV), wherein: X is selected from the group consisting of CH₂, S, or SO; R is selected from the group consisting of hydrogen, ethyl, or methyl; the stereochemistry at the 2, 2′, and 5 is selected from the group consisting of S,S; S,R; R,S; R,R; S,S-rac; S,S,S; and S,S; and n is 1 or 2.

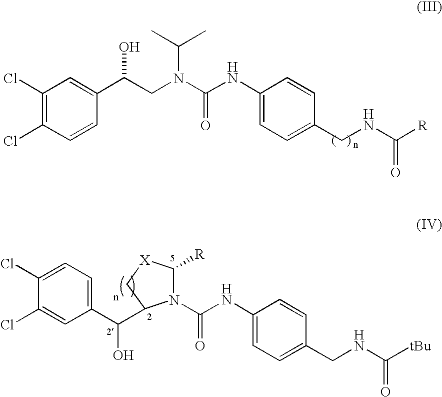

19. The method of claim 14, wherein the PAR1 antagonist is selected from the group consisting of:
(a) a compound of Formula (V); and
(b) derivatives of the compound of Formula (V) that have the structure of Formula (VI) wherein R is selected from the group consisting of 3-methyl, 4-methyl, 3-methoxy, 4-methoxy, or 3,5-difluoro.

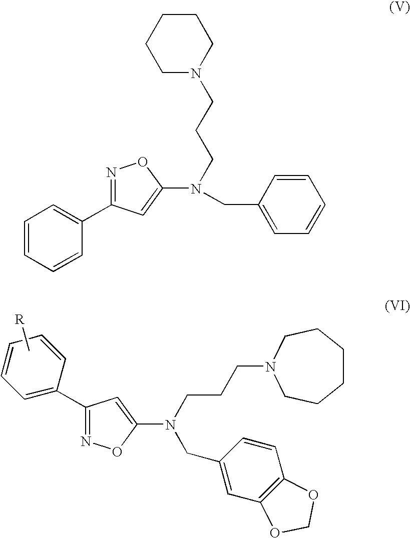

20. The method of claim 14 wherein the PAR1 antagonist is a derivative of a 1,3-dihydrobenzoimidazolamine analogue of Formula (VII).

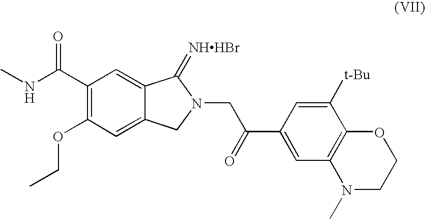

21. The method of claim 14 wherein the PAR1 antagonist is a pyrroloquinazoline of Formula (VIII).

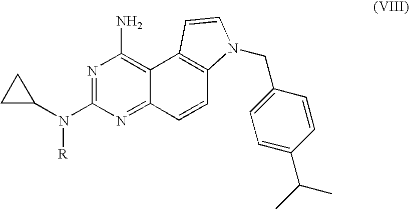

27. The method of claim 18 wherein the PAR1 antagonist is (N-3-cyclopropyl-7-{[4-(1-methylethyl)phenyl]methyl}-7H-pyrrolo[3,2-f]quinazoline-1,3-diamine).

28. The method of claim 18 wherein the PAR1 antagonist is a benzimidazole derivative of Formula (IX) in which R is selected from the group consisting of hydrogen, methyl, n-butyl, benzyl, and 4-methylbenzyl and R₁ is selected from the group consisting of hydrogen and t-butyl.

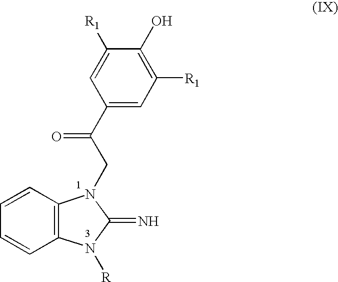

22. The method of claim 14 wherein the PAR1 antagonist is a derivative of himbacine.

23. The method of claim 14 wherein the PAR1 antagonist is an iminopyrrolidine derivative of Formula (XI) wherein: ring A indicates a pyrrolidine ring; ring B indicates a benzene ring or a pyridine ring; R¹⁰¹, R¹⁰², and R¹⁰³ each independently indicate, identically or differently, a hydrogen atom, a halogen atom, a C₁-C₆ alkyl group, or a C₁-C₆ alkoxy-substituted C₁-C₆ alkyl group; R⁵ indicates a hydrogen atom; R⁶ indicates a hydrogen atom, a C₁-C₆ alkyl group, or a C₁-C₆ alkyloxy-carbonyl group; Y¹ indicates a single valence bond or a —CH₂— group; Y² indicates a single valence bond or a —CO— group; Ar¹ indicates a hydrogen atom or a group represented by Formula (XII), wherein, in Formula (XII), R¹⁰, R¹¹, R¹², R¹³, and R¹⁴ independently indicate, identically, or differently, a hydrogen atom, a C₁-C₆ alkyl group, or a C₁-C₆ alkoxy group, a morpholinyl group, a piperazinyl group which can be substituted or unsubstituted, or a piperidinyl group which can be substituted or unsubstituted, and R¹¹, R¹², and R¹³ can form a 5-8 membered heterocycle.

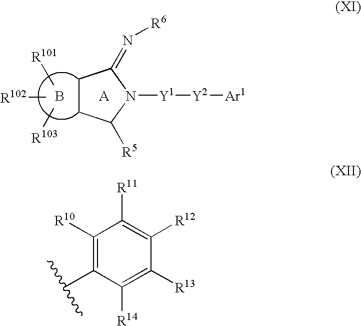

24. The method of claim 14 wherein the PAR1 antagonist is selected from the group consisting of an analogue or derivative of a 2-iminopyrrolidine compound selected from the group consisting of 2-[2-(3,5-di-t-butyl-4-hydroxy-phenyl)-2-oxo-ethyl]-6-ethoxy-3-imino-2,-3-dihydro-1H-isoindole-5-carboxylic acid methylamide trifluoroacetate; 2-[2-(3,5-di-t-butyl-4-hydroxy-phenyl)-2-oxo-ethyl]-6-dimethylamino-3-imino-2,3-dihydro-1H-isoindole-5-carboxylic acid methylamide hydrobromide; 1-(3,5-di-t-butyl-4-hydroxy-phenyl)-2-(7-fluoro-1-imino-5,6-dimethoxy 1,3-dihydro-isoindol-2-yl)-ethanone hydrobromide; 1-(8-t-butyl-4-methyl-3,4-dihydro-2H-benzo[1,4]oxazin-6-yl)-2-(5,6-diethoxy-7-fluoro-1-imino-1,3-dihydro-isoindol-2-yl)-ethanone hydrobromide; 2-[2-(3-t-butyl-4,5-dimethoxy-phenyl)-2-oxo-ethyl]-6-ethoxy-3-imino-2,-3-dihydro-1H-isoindole-5-carboxylic acid methylamide hydrobromide; 1-(3,5-di-t-butyl-4-hydroxy-phenyl)-2-(5-ethoxy-7-fluoro-1-imino-6-methoxy-1,3-dihydro-isoindol-2-yl)-ethanone hydrobromide; 2-[2-(3-t-butyl-4-hydroxy-5-isopropoxy-phenyl)-2-oxo-ethyl]-6-ethoxy-3-imino-2,3-dihydro-1H-isoindole-5-carboxylic acid methylamide hydrobromide; 6-[2-(8-t-butyl-4-methyl-3,4-dihydro-2H-benzo[1,4]oxazin-6-yl)-2-oxo-ethyl]-3-ethoxy-7-imino-6,7-dihydro-5H-pyrrolo[3,4-b]pyridine-2-carboxylic acid methylamide hydrochloride; 2-[2-(7-t-butyl-3-methyl-3H-benzimidazol-5-yl)-2-oxo-ethyl]-6-ethoxy-3-imino-2,3-dihydro-1H-isoindole-5-carboxylic acid methylamide hydrobromide; 2-[2-(3-t-butyl-5-dimethylamino-4-hydroxy-phenyl)-2-oxo-ethyl]-6-ethoxy-3-imino-2,3-dihydro-1H-isoindole-5-carboxylic acid methylamide hydrobromide; 5-{2-tert-butyl-4-[2-(2-cyclopropyl-7-imino-5,7-dihydro-pyrrolo[3,4-b]pyridin-6-yl)-acetyl]-6-(pyrrolidin-1-yl)-phenoxy}-pentanoic acid trifluoroacetate; 5-(2-t-butyl-4-[2-(7-fluoro-1-imino-5,6-dimethoxy-1,3-dihydro-isoindol-2-yl)-acetyl]-6-(pyrrolidin-1-yl)-phenoxy}-pentanoic acid trifluoroacetate; 2-[2-(3-t-butyl-5-dimethylamino-4-methoxy-phenyl)-2-oxo-ethyl]-6-ethoxy-3-imino-2,3-dihydro-1H-isoindole-5-carboxylic acid methylamide hydrobromide; 2-(1-(3-t-butyl-5-[2-(7-fluoro-1-imino-5,6-dimethoxy-1,3-dihydro-isoindol-2-yl)-acetyl]-2-methoxy-phenyl)-pyrrolidin-3-yloxy)-butyric acid trifluoroacetate; 2-(1-{3-t-butyl-5-[2-(7-fluoro-1-imino-5,6-dimethoxy-1,3-dihydro-isoindol-2-yl)-acetyl]-2-methoxy-phenyl)-pyrrolidin-3-yloxy)-butyric acid trifluoroacetate; 1-(3-t-butyl-5-dimethylamino-4-methoxy-phenyl)-2-(7-fluoro-1-imino-5,6-dimethoxy-1,3-dihydro-isoindol-2-yl)-ethanone hydrobromide; 2-[2-(3-t-butyl-5-dimethylamino-4-methoxy-phenyl)-2-oxo-ethyl]-6-dimethylamino-3-imino-2,3-dihydro-1H-isoindole-5-carboxylic acid methylamide hydrobromide; 6-[2-(3-t-butyl-5-dimethylamino-4-methoxy-phenyl)-2-oxo-ethyl]-3-ethoxy-7-imino-6,7-dihydro-5H-pyrrolo[3,4-b]pyridine-2-carboxylic acid methylamide hydrochloride; 2-[2-(3-t-butyl-5-ethoxy-4-methoxy-phenyl]-2-oxo-ethyl]-6-dimethylamino-3-imino-2,3-dihydro-1H-isoindole-5-carboxylic acid methylamide hydrobromide; 2-[2-(3-t-butyl-5-ethoxy-4-methoxy-phenyl)-2-oxo-ethyl]-6-ethoxy-3-imino-2,3-dihydro-1H-isoindole-5-carboxylic acid methylamide hydrobromide; 1-(3-t-butyl-5-ethoxy-4-methoxy-phenyl)-2-(5,6-diethoxy-7-fluoro-1-imino-1,3-dihydro-isoindol-2-yl)-ethanone hydrobromide; 6-[2-(3-t-butyl-5-ethoxy-4-methoxy-phenyl)-2-oxo-ethyl]-3-ethoxy-7-imino-6,7-dihydro-5H-pyrrolo[3,4-b]pyridine-2-carboxylic acid methylamide trifluoroacetate; {3-t-butyl-5-[2-(7-fluoro-1-imino-5,6-dimethoxy-1,3-dihydro-isoindol-2-yl)-acetyl]-2-methoxy-phenoxy}-acetonitrile hydrobromide; 4-(3-t-butyl-5-[2-(7-fluoro-1-imino-5,6-dimethoxy-1,3-dihydro-isoindol-2-yl)-acetyl]-2-methoxy-phenoxy)-butyronitrile hydrobromide; 2-[2-(3-t-butyl-5-cyanomethoxy-4-methoxy-phenyl)-2-oxo-ethyl]-6-dimethylamino-3-imino-2,3-dihydro-1H-isoindole-5-carboxylic acid methylamide hydrobromide; 2-{2-[3-t-butyl-5-(3-cyano-propoxy)-4-methoxy-phenyl]-2-oxo-ethyl)-6-dimethylamino-3-imino-2,3-dihydro-1H-isoindole-5-carboxylic acid methylamide hydrobromide; 2-[2-(8-t-butyl-4-cyanomethyl-3,4-dihydro-2H-benzo[1,4]oxazin-6-yl)-2-oxo-ethyl]-6-ethoxy-3-imino-2,3-dihydro-1H-isoindole-5-carboxylic acid methylamide trifluoroacetate; 6-[2-(8-t-butyl-4-cyanomethyl-3,4-dihydro-2H-benzo[1,4]oxazin-6-yl)-2-oxo-ethyl]-3-ethoxy-7-imino-6,7-dihydro-5H-pyrrolo[3,4-b]pyridine-2-carboxylic acid methylamide trifluoroacetate; (8-t-butyl-6-[2-(7-fluoro-1-imino-5,6-dimethoxy-1,3-dihydro-isoindol-2-yl)-acetyl]-2,3-dihydro-benzo[1,4]oxazin-4-yl)-acetonitrile trifluoroacetate; {8-t-butyl-6-[2-(5,6-diethoxy-7-fluoro-1-imino-1,3-dihydro-isoindol-2-yl)-acetyl]-2,3-dihydro-benzo[1,4]oxazin-4-yl}-acetonitrile trifluoroacetate; (8-t-butyl-6-[2-(2-cyclopropyl-7-imino-5,7-dihydro-pyrrolo[3,4-b]pyridin-6-yl)-acetyl]-2,3-dihydro-benzo[1,4]oxazin-4-yl)-acetonitrile trifluoroacetate; 2-[2-(8-t-butyl-4-cyanomethyl-3,4-dihydro-2H-benzo[1,4]oxazin-6-yl)-2-oxo-ethyl]-6-dimethylamino-3-imino-2,3-dihydro-1H-isoindole-5-carboxylic acid methylamide trifluoroacetate; 2-[2-(3-t-butyl-5-cyanomethoxy-4-methoxy-phenyl)-2-oxo-ethyl]-6-ethoxy-3-imino-2,3-dihydro-1H-isoindole-5-carboxylic acid methylamide hydrobromide; 2-(2-[3-t-butyl-5-(3-cyano-propoxy)-4-methoxy-phenyl]-2-oxo-ethyl}-6-ethoxy-3-imino-2,3-dihydro-1H-isoindole-5-carboxylic acid methylamide hydrobromide; 1-(3-t-butyl-S-ethoxy-4-methoxy-phenyl)-2-(7-fluoro-1-imino-5,6-dimethoxy-1,3-dihydro-isoindol-2-yl)-ethanone hydrobromide; 2-[2-(3-t-butyl-4-methoxy-5-morpholino-phenyl)-2-oxo-ethyl]-6-ethoxy-3-imino-2,3-dihydro-1H-isoindole-5-carboxylic acid methylamide hydrobromide; 1-(3-t-butyl-4-methoxy-5-morpholino-phenyl)-2-(7-fluoro-1-imino-5,6-di-methoxy-1,3-dihydro-isoindol-2-yl)-ethanone hydrobromide; 1-(3-t-butyl-4-methoxy-5-morpholino-phenyl)-2-(2-cyclopropyl-7-imino-5-,7-dihydro-pyrrolo[3,4-b]pyridin-6-yl)-ethanone hydrobromide; (3-t-butyl-5-[2-(5,6-diethoxy-7-fluoro-1-imino-1,3-dihydro-isoindol-2-yl)-acetyl]-2-methoxy-phenoxy)-acetonitrile hydrobromide; 4-{3-t-butyl-5-[2-(5,6-diethoxy-7-fluoro-1-imino-1,3-dihydro-isoindol-2-yl)-acetyl]-2-methoxy-phenoxy)-butyronitrile hydrobromide; 4-{3-t-butyl-5-[2-(2-cyclopropyl-7-imino-5,7-dihydro-pyrrolo[3,4-b]pyridin-6-yl)-acetyl]-2-methoxy-phenoxy}-butyronitrile hydrobromide; 1-(3-t-butyl-5-dimethylamino-4-methoxy-phenyl)-2-(5,6-diethoxy-7-fluor-o-1-imino-1,3-dihydro-isoindol-2-yl)-ethanone hydrobromide; 1-(3-t-butyl-4-methoxy-5-morpholino-phenyl)-2-(5,6-diethoxy-7-fluoro-1-imino-1,3-dihydro-isoindol-2-yl)-ethanone hydrobromide; 6-[2-(3-t-butyl-4-methoxy-5-morpholino-phenyl)-2-oxo-ethyl]-3-ethoxy-7-imino-6,7-dihydro-5H-pyrrolo[3,4-b]pyridine-2-carboxylic acid methylamide hydrobromide; 2-t-butyl-4-[2-(5-ethoxy-1-imino-6-methylcarbamoyl-1,3-dihydro-isoindol-2-yl)-acetyl]-phenyl methanesulfonate hydrobromide; 2-t-butyl-4-[2-(5-dimethylamino-1-imino-6-methylcarbamoyl-1,3-dihydroisoindol-2-yl)-acetyl]-phenyl methanesulfonate hydrobromide; 2-t-butyl-4-[2-(3-ethoxy-7-imino-2-methylcarbamoyl-5,7-dihydro-pyrrolo-[3,4-b]pyridin-6-yl)-acetyl]-phenyl methanesulfonate hydrobromide; 2-[2-(3-t-butyl-4-cyanomethoxy-5-dimethylamino-phenyl)-2-oxo-ethyl]-6-dimethylamino-3-imino-2,3-dihydro-1H-isoindole-5-carboxylic acid methylamide hydrobromide; 2-[2-(3-t-butyl-4-methoxy-5-(pyrrolidin-1-yl)-phenyl)-2-oxo-ethyl]-6-ethoxy-3-imino-2,3-dihydro-1H-isoindole-5-carboxylic acid methylamide hydrobromide; 1-(3-t-butyl-4-methoxy-5-(pyrrolidin-1-yl)phenyl)-2-(5,6-diethoxy-7-fluoro-1-imino-1,3-dihydro-isoindol-2-yl)-ethanone hydrobromide; 2-[2-(3-t-butyl-4-methoxy-5-(pyrrolidin-1-yl)-phenyl)-2-oxo-ethyl]-6-dimethylamino-3-imino-2,3-dihydro-1H-isoindole-5-carboxylic acid methylamide hydrobromide; 1-(3-t-butyl-4-methoxy-5-(pyrrolidin-1-yl)-phenyl)-2-(2-cyclopropyl-7-imino-5,7-dihydro-pyrrolo[3,4-b]pyridin-6-yl)-ethanone hydrobromide; 1-(3-t-butyl-4-methoxy-5-(pyrrolidin-1-yl)-phenyl)-2-(7-fluoro-1-imino-5,6-dimethoxy-1,3-dihydro-isoindol-2-yl)-ethanone hydrobromide; 2-[2-(3-t-butyl-5-isopropoxy-4-methoxy-phenyl)-2-oxo-ethyl]-6-ethoxy-3-imino-2,3-dihydro-1H-isoindole-5-carboxylic acid methylamide hydrobromide; 1-(3-t-butyl-5-isopropoxy-4-methoxy-phenyl)-2-(5,6-diethoxy-7-fluoro-1-imino-1,3-dihydro-isoindol-2-yl)-ethanone hydrobromide; 1-(3-t-butyl-5-isopropoxy-4-methoxy-phenyl)-2-(7-fluoro-1-imino-5,6-di-methoxy-1,3-dihydro-isoindol-2-yl)-ethanone hydrobromide; ethyl 2-{8-t-butyl-6-[2-(5-ethoxy-1-imino-6-methylcarbamoyl-1,3-dihydro-isoindol-2-yl)-acetyl}-2,3-dihydro-benzo[1,4]oxazin-4-yl)-propionate hydrochloride; ethyl 2-{8-t-butyl-6-[2-(3-ethoxy-7-imino-2-methylcarbamoyl-5,7-dihydro-pyrrolo[3,4-b]pyridin-6-yl)-acetyl]-2,3-dihydro-benzo[1,4]oxazin-4-yl}-propionate hydrochloride; 2-[2-(3-dimethylamino-5-isopropyl-4-methoxy-phenyl)-2-oxo-ethyl]-6-ethoxy-3-imino-2,3-dihydro-1H-isoindole-5-carboxylic acid methylamide hydrobromide; 2-(5,6-diethoxy-7-fluoro-1-imino-1,3-dihydro-isoindol-2-yl)-1-(3-dimethyl-amino-5-isopropyl-4-methoxy-phenyl)-ethanone hydrobromide; 2-[2-(3-t-butyl-4-methoxy-5-methylamino-phenyl)-2-oxo-ethyl]-6-ethoxy-3-imino-2,3-dihydro-1H-isoindole-5-carboxylic acid methylamide hydrobromide; 6-[2-(3-t-butyl-5-isopropoxy-4-methoxy-phenyl)-2-oxo-ethyl]-3-ethoxy-7-imino-6,7-dihydro-5H-pyrrolo[3,4-b]pyridine-2-carboxylic acid methylamide trifluoroacetate; 2-[2-(3-t-butyl-5-isopropoxy-4-methoxy-phenyl)-2-oxo-ethyl]-6-dimethyl-amino-3-imino-2,3-dihydro-1H-isoindole-5-carboxylic acid methylamide trifluoroacetate; 2-{2-[3-t-butyl-5-(4-cyano-piperidin-1-yl)-4-methoxy-phenyl]-2-oxo-ethyl}-6-ethoxy-3-imino-2,3-dihydro-1H-isoindole-5-carboxylic acid methylamide hydrobromide; 1-(3-t-butyl-4-hydroxy-phenyl)-2-(5,6-diethoxy-7-fluoro-1-imino-1,3-dihydro-isoindol-2-yl)-ethanone hydrobromide; 2-{8-t-butyl-6-[2-(5-ethoxy-1-imino-6-methylcarbamoyl-1,3-dihydro-isoindol-2-yl)-acetyl}-2,3-dihydro-benzo[1,4]oxazin-4-yl}-2-methyl-propanoic acid hydrochloride; 2-{8-t-butyl-6-[2-(3-ethoxy-7-imino-2-methylcarbamoyl-5,7-dihydro-pyrrolo[3,4-b]pyridin-6-yl)-acetyl]-2,3-dihydro-benzo[1,4]oxazin-4-yl}-2-methyl-propanoic acid hydrochloride; 2-t-butyl-6-dimethylamino-4-[2-(5-ethoxy-1-imino-6-methylcarbamoyl-1,3-dihydro-isoindol-2-yl)-acetyl]-phenyl acetate hydrobromide; 2-{2-[3-t-butyl-4-methoxy-5-(2-oxo-oxazolidin-3-yl)-phenyl]-2-oxo-ethyl}-6-ethoxy-3-imino-2,3-dihydro-1H-isoindole-5-carboxylic acid methylamide hydrobromide; 2-t-butyl-4-[2-(5,6-diethoxy-7-fluoro-1-imino-1,3-dihydro-isoindol-2-yl)-acetyl]-phenyl acetate hydrobromide; 2-t-butyl-4-[2-(5-ethoxy-1-imino-6-methylcarbamoyl-1,3-dihydro-isoindol-2-yl)-acetyl]-phenyl acetate hydrobromide; 1-{3-t-butyl-5-[2-(5,6-diethoxy-7-fluoro-1-imino-1,3-dihydro-isoindol-2-yl)-acetyl]-2-methoxy-phenyl}-piperidin-4-one hydrobromide; 1-(3-t-butyl-5-dimethylamino-4-methoxy-phenyl)-2-(5-ethoxy-7-fluoro-1-imino-6-methoxy-1,3-dihydro-isoindol-2-yl)-ethanone hydrobromide; 2-{2-[3-t-butyl-5-(ethyl-methyl-amino)-4-methoxy-phenyl]-2-oxo-ethyl}-6-ethoxy-3-imino-2,3-dihydro-1H-isoindole-5-carboxylic acid methylamide hydrobromide; 6-{2-[3-t-butyl-5-(ethyl-methyl-amino)-4-methoxy-phenyl]-2-oxo-ethyl}-3-ethoxy-7-imino-6,7-dihydro-5H-pyrrolo[3,4-b]pyridine-2-carboxylic acid methylamide trifluoroacetate; 2-t-butyl-4-[2-(5,6-diethoxy-7-fluoro-1-imino-1,3-dihydro-isoindol-2-yl)-acetyl]-6-dimethylamino-phenyl methanesulfonate hydrobromide; 1-[3-t-butyl-5-(4-hydroxy-piperidin-1-yl)-4-methoxy-phenyl]-2-(5,6-diethoxy-7-fluoro-1-imino-1,3-dihydro-isoindol-2-yl)-ethanone hydrobromide; 6-[(2-[3-t-butyl-5-(4-hydroxy-piperidin-1-yl)-4-methoxy-phenyl]-2-oxo-ethyl]-3-ethoxy-7-imino-6,7-dihydro-5H-pyrrolo[3,4-b]pyridine-2-carboxylic acid methylamide trifluoroacetate; 1-[3-t-butyl-5-(4-hydroxy-piperidin-1-yl)-4-methoxy-phenyl]-2-(7-fluoro-1-imino-5,6-dimethoxy-1,3-dihydro-isoindol-2-yl)-ethanone hydrobromide; 1-(3-t-butyl-5-dimethylamino-4-hydroxy-phenyl)-2-(5,6-diethoxy-7-fluoro-1-imino-1,3-dihydro-isoindol-2-yl)-ethanone hydrobromide; 6-[2-(3-t-butyl-5-dimethylamino-4-ethoxy-phenyl)-2-oxo-ethyl]-3-ethoxy-7-imino-6,7-dihydro-5H-pyrrolo[3,4-b]pyridine-2-carboxylic acid methylamide trifluoroacetate; 2-[2-(3-t-butyl-5-dimethylamino-4-methoxy-phenyl)-2-oxo-ethyl]-6-dimethylamino-3-imino-2,3-dihydro-1H-isoindole-5-carboxylic acid methylamide hydrobromide; 1-(3-t-butyl-4-methoxy-5-methylamino-phenyl)-2-(5,6-diethoxy-7-fluoro-1-imino-1,3-dihydro-isoindol-2-yl)-ethanone hydrobromide; 1-[3-t-butyl-5-(4-hydroxy-piperidin-1-yl)-4-methoxy-phenyl]-2-(5-ethoxy-7-fluoro-1-imino-6-methoxy-1,3-dihydro-isoindol-2-yl hydrobromide; 2-(5,6-diethoxy-7-fluoro-1-imino-1,3-dihydro-isoindol-2-yl)-1-[3-dimethyl-amino-5-(1-fluoro-1-methyl-ethyl)-4-methoxy-phenyl]-ethanone hydrobromide; 1-[3-t-butyl-5-(3-hydroxy-pyrrolidin-1-yl)-4-methoxy-phenyl]-2-(5,6-di-ethoxy-7-fluoro-1-imino-1,3-dihydro-isoindol-2-yl)-ethanone trifluoroacetate; 2-[2-(3-t-butyl-5-ethylamino-4-methoxy-phenyl)-2-oxo-ethyl]-6-ethoxy-3-imino-2,3-dihydro-1H-isoindole-5-carboxylic acid methylamide hydrobromide; 2-[2-[(3-t-butyl-5-ethylamino-4-methoxy-phenyl)-2-oxo-ethyl]-6-ethoxy-3-imino-2,3-dihydro-1H-isoindole-5-carboxylic acid methylamide hydrobromide; 1-(3-t-butyl-5-ethoxy-4-hydroxy-phenyl)-2-(5,6-diethoxy-7-fluoro-1-imino-1,3-dihydro-isoindol-2-yl)-ethanone hydrobromide; 2-[2-(3-t-butyl-5-ethoxy-4-hydroxy-phenyl)-2-oxo-ethyl]-6-ethoxy-3-imino-2,3-dihydro-1H-isoindole-5-carboxylic acid methylamide hydrobromide; 2-t-butyl-4-[2-(5,6-diethoxy-7-fluoro-1-imino-1,3-dihydro-isoindol-2-y-l)-acetyl]-6-ethoxy-phenyl ethyl-carbamate hydrobromide; 2-t-butyl-6-ethoxy-4-[2-(5-ethoxy-1-imino-6-methylcarbamoyl-1,3-dihydro-isoindol-2-yl)-acetyl]-phenyl ethyl-carbamate hydrobromide; 2-t-butyl-6-(3-cyano-propoxy)-4-[2-(5,6-diethoxy-7-fluoro-1-imino-1,3-dihydro-isoindol-2-yl)-acetyl]-phenyl methanesulfonate hydrobromide; 1-(3-t-butyl-4-methoxy-5-piperazin-1-yl-phenyl)-2-(5,6-diethoxy-7-fluoro-1-imino-1,3-dihydro-isoindol-2-yl)-ethanone dihydrochloride; 2-(2-{3-t-butyl-4-methoxy-5-[(2-methoxyethyl)-methylamino]-phenyl}-2-oxo-ethyl)-6-dimethylamino-3-imino-2,3-dihydro-1H-isoindole-5-carboxylic acid methylamide hydrobromide; 1-[3-t-butyl-5-(2-hydroxyethylamino)-4-methoxyphenyl]-2-(5,6-diethoxy-7-fluoro-1-imino-1,3-dihydro-isoindol-2-yl)-ethanone hydrochloride; 1-{3-t-butyl-5-[(2-hydroxyethyl)-methylamino]-4-methoxy-phenyl}-2-(5,6-diethoxy-7-fluoro-1-imino-1,3-dihydro-isoindol-2-yl)-ethanone dihydrochloride; 2-(2-[3-t-butyl-5-(3,4-dihydroxy-pyrrolidin-1-yl)-4-methoxy-phenyl]-2-oxo-ethyl}-6-ethoxy-3-imino-2,3-dihydro-1H-isoindole-5-carboxylic acid methylamide trifluoroacetate; 1-[3-t-butyl-5-(3-hydroxy-4-methoxy-pyrrolidin-1-yl)-4-methoxy-phenyl]-2-(5,6-diethoxy-7-fluoro-1-imino-1,3-dihydro-isoindol-2-yl)-ethanone trifluoroacetate; (3-t-butyl-5-[2-(5,6-diethoxy-7-fluoro-1-imino-1,3-dihydro-isoindol-2-yl)-acetyl]-2-methoxy-phenylamino}-acetonitrile hydrobromide; 1-(3-t-butyl-4-hydroxy-5-morpholino-phenyl)-2-(5,6-diethoxy-7-fluoro-1-imino-1,3-dihydro-isoindol-2-yl)-ethanone hydrochloride; 1-{3-t-butyl-5-[ethyl-(2-hydroxyethyl)-amino]-4-methoxy-phenyl}-2-(5,6-diethoxy-7-fluoro-1-imino-1,3-dihydro-isoindol-2-yl)-acetyl hydrochloride; (4-{3-t-butyl-5-[2-(5,6-diethoxy-7-fluoro-1-imino-1,3-dihydro-isoindol-2-yl)-acetyl]-2-methoxy-phenyl}-piperazin-1-yl)-acetonitrile hydrobromide; 2-(2-{3-t-butyl-5-[(2-hydroxyethyl)-methylamino]-4-methoxy-phenyl}-2-oxo-ethyl)-6-dimethylamino-3-imino-2,3-dihydro-1H-isoindole-5-carboxylic acid methylamide dihydrochloride; 1-{3-t-butyl-5-[(3-hydroxypropyl)-methylamino]-4-methoxy-phenyl}-2-(5,-6-diethoxy-7-fluoro-1-imino-1,3-dihydro-isoindol-2-yl)-ethanone dihydrochloride; 1-{3-t-butyl-5-[(2-hydroxyethyl)-(2-methoxyethyl)-amino]-4-methoxy-phenyl}-2-(5,6-diethoxy-7-fluoro-1-imino-1,3-dihydro-isoindol-2-yl)-ethanone dihydrochloride; 1-[3-t-butyl-5-(3-hydroxy-4-methoxy-pyrrolidin-1-yl)-4-methoxy-phenyl]-2-(5,6-diethoxy-7-fluoro-1-imino-1,3-dihydro-isoindol-2-yl)-ethanone trifluoroacetate; 1-(3-amino-5-t-butyl-4-methoxy-phenyl)-2-(5,6-diethoxy-7-fluoro-1-imino-1,3-dihydro-isoindol-2-yl)-ethanone hydrobromide; 2-[2-(3-t-butyl-5-isopropylamino-4-methoxy-phenyl)-2-oxo-ethyl]-6-ethoxy-3-imino-2,3-dihydro-1H-isoindole-5-carboxylic acid methylamide hydrobromide; 1-[3-(4-acetyl-piperazin-1-yl)-5-t-butyl-4-methoxy-phenyl]-2-(5,6-diethoxy-7-fluoro-1-imino-1,3-dihydro-isoindol-2-yl)-ethanone hydrobromide; 1-[3-t-butyl-5-(3-hydroxy-4-methoxy-pyrrolidin-1-yl)-4-methoxy-phenyl]-2-(5,6-diethoxy-7-fluoro-1-imino-1,3-dihydro-isoindol-2-yl)-ethanone trifluoroacetate; 1-[3-t-butyl-5-(3,4-dimethoxy-pyrrolidin-1-yl)-4-methoxy-phenyl]-2-(5,6-diethoxy-7-fluoro-1-imino-1,3-dihydro-isoindol-2-yl)-ethanone hydrobromide; (4-{3-t-butyl-5-[2-(5,6-diethoxy-7-fluoro-1-imino-1,3-dihydro-isoindol-2-yl)-acetyl]-2-methoxy-phenyl}-piperazin-1-yl)-acetic acid dihydrochloride; 1-(3-t-butyl-5-[4-(2-hydroxy-acetyl)-piperazin-1-yl]-4-methoxy-phenyl}-2-(5,6-diethoxy-7-fluoro-1-imino-1,3-dihydro-isoindol-2-yl)-ethanone hydrobromide; 4-{3-t-butyl-5-[2-(5,6-diethoxy-7-fluoro-1-imino-1,3-dihydro-isoindol-2-yl)-acetyl]-2-methoxy-phenyl}-piperazine-1-carboxylic acid ethylamide hydrobromide; ethyl (4-{3-t-butyl-5-[2-(5,6-diethoxy-7-fluoro-1-imino-1,3-dihydro-isoindol-2-yl)-acetyl]-2-methoxy-phenyl)-piperazin-1-yl)-acetate dihydrochloride; 1-(3-t-butyl-4-methoxy-5-[4-(2-methoxy-acetyl)-piperazin-1-yl]-phenyl}-2-(5,6-diethoxy-7-fluoro-1-imino-1,3-dihydro-isoindol-2-yl)-ethanone hydrobromide; 1-(4-{3-t-butyl-5-[2-(5,6-diethoxy-7-fluoro-1-imino-1,3-dihydro-isoindol-2-yl)-acetyl]-2-methoxy-phenyl}-piperazin-1-yl)-propan-1-one hydrobromide; 1-[3-t-butyl-5-(3-ethoxy-4-hydroxy-pyrrolidin-1-yl)-4-methoxy-phenyl]-2-(5,6-diethoxy-7-fluoro-1-imino-1,3-dihydro-isoindol-2-yl)-ethanone trifluoroacetate, 1-(3,5-di-t-butyl-4-hydroxy-phenyl)-2-(7-imino-2-methyl-5,7-dihydro-pyrrolo[3,4-b]pyridin-6-yl)-ethanone hydrochloride; 1-(3,5-di-t-butyl-4-hydroxy-phenyl)-2-(3-ethoxy-7-imino-2,4-dimethyl-5,7-dihydro-pyrrolo[3,4-b]pyridin-6-yl)-ethanone hydrobromide; 2-[2-(3,5-di-t-butyl-4-hydroxy-phenyl)-2-oxo-ethyl]-6-ethoxy-3-imino-2,3-dihydro-1H-isoindole-5-carboxamide hydrobromide; 2-[2-(8-t-butyl-4-methyl-3,4-dihydro-2H-benzo[1,4]oxazin-6-yl)-2-oxo-ethyl]-6-ethoxy-3-imino-2,3-dihydro-1H-isoindole-5-carboxylic acid methylamide hydrobromide; 6-[2-(3,5-di-t-butyl-4-hydroxy-phenyl)-2-oxo-ethyl]-3-ethoxy-7-imino-6,7-dihydro-5H-pyrrolo[3,4-b]pyridine-2-carboxylic acid methylamide hydrochloride; 1-(3,5-di-t-butyl-4-hydroxy-phenyl)-2-(2-ethyl-7-imino-5,7-dihydro-pyrrolo[3,4-b]pyridin-6-yl)-ethanone hydrobromide; 2-(2-cyclopropyl-7-imino-5,7-dihydro-pyrrolo[3,4-b]pyridin-6-yl)-ethanone)-1-(3,5-di-t-butyl-4-hydroxy-phenyl)-ethanone hydrobromide; 2-[2-(8-t-butyl-3-oxo-3,4-dihydro-2H-benzo[1,4]oxazin-6-yl)-2-oxo-ethyl]-6-ethoxy-3-imino-2,3-dihydro-1H-isoindole-5-carboxylic acid methylamide hydrobromide; and 1-(3-t-butyl-5-isopropylamino-4-methoxy-phenyl)-2-(5,6-diethoxy-7-fluoro-1-imino-1,3-dihydro-isoindol-2-yl)-ethanone hydrobromide.

25. The method of claim 14 wherein the PAR1 antagonist is a peptide.

26. The method of claim 25 wherein the peptide is a peptide of a sequence selected from the group consisting of MSRPACPN (SEQ ID NO: 1) and AFLARAA (SEQ ID NO: 2).

27. The method of claim 14 wherein the PAR1 antagonist is eryloside F.

28. The method of claim 14 wherein the PAR1 antagonist is a peptidomimetic.

29. The method of claim 28 wherein the peptidomimetic is an azole PAR1 antagonist of Formula (XXII) wherein;
(a) A₁ is selected from the group consisting of:
(i) an amino acid residue selected from the group consisting of Sar, Gly, His, His(CH₂ Ph), Ile, Ser, Thr, β-Ala, or Ala;
(ii) a C₂-C₆-acyl group; and
(iii) a C₁-C₈-alkyl group
(b) A₂ is selected from the group consisting of:
(i) an alkyl amino acid residue selected from the group consisting of Cha, Leu, Ile, Asp, and Glu; and
(ii) an amino alkyl amino acid residue selected from the group consisting of Lys, His, Orn, homoArg and Arg;
(c) A₃ is an amino alkyl amino acid residue selected from the group consisting of Lys, His, Orn, Arg and homoArg;
(d) A₄ is selected from the group consisting of:
(i) an arylalkyl residue selected from Phe and Tyr; and
(ii) an aralkylamino group selected from the group consisting of a benzylamino group and a phenethylamino group;
(e) R₁ is selected from H or alkyl;
(f) R₂ is a moiety selected from the group consisting of an aryl, substituted aryl, heteroaryl, substituted heteroaryl, aralkyl and substituted aralkyl group;
(g) R₃ is selected from the group consisting of H and alkyl; and
(h) X is selected from the group consisting of S, O, and NR₄, wherein R₄ is selected from the group consisting of H and alkyl;
and the pharmaceutically acceptable salts thereof.

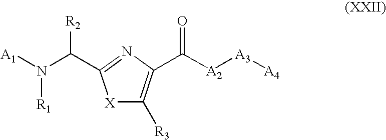

30. The method of claim 14 wherein the PAR1 antagonist is a compound of Formula (XXIII) wherein: R¹ and R³ are amide linkages, N-alkylamide linkages, or isosteric replacements of such linkages; R² is either a neutral amino acid side chain or a hydrophobic radical; R⁴ is a hydrophobic radical; R⁵ is CO, CH², or SO; X is either: (1) a moiety of Formula (XXIV) in which R⁶ and R⁷ are the same or different and are hydrogen, alkyl, (cycloalkyl)alkyl, alkoxyalkyl, alkylthioalkyl, or aralkyl; or (2) a hydrophobic residue containing at least one aryl moiety; and R⁸ is a hydrophobic radical or is a hydrophobic residue; Y is alkoxy, hydroxyl, amino, alkylamino, or dialkylamino, in which any of the alkyl groups can be substituted with a basic moiety; Z is an amino acid or peptide residue; and m is 0 or 1.

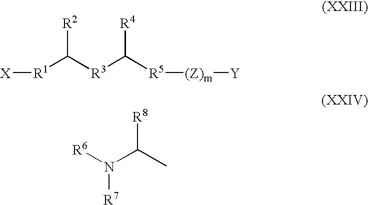

31. The method of claim 28 wherein the PAR1 antagonist is a peptidomimetic selected from the group consisting of (αS)—N-[(1S)-3-amino-1-[[(phenylmethyl)amino]carbonyl]propyl]-α-[[[[[1-(2,6-dichlorophenyl)methyl]-3-(1-pyrrolidinylmethyl)-1H-indol-6-yl]amino]carbonyl]amino]-3,4-difluorobenzenepropanamide and a derivative thereof in which the substituted indole ring is replaced by an identically substituted indazole ring.

32. The method of claim 14 wherein the PAR1 antagonist is a pepducin.

33. The method of claim 32 wherein the pepducin is a pepducin comprising: (1) a first domain that includes either extracellular or intracellular portions of PAR1, the first domain not comprising a native extracellular ligand of PAR1; and (2) at least a second domain, attached to the first domain, wherein the second domain is a hydrophobic moiety which is either naturally or non-naturally occurring.

34. The method of claim 33 wherein the pepducin is selected from the group consisting of pal-RCLSSSAVANRS (SEQ ID NO: 3) and pal-KKSRALF (SEQ ID NO: 4).

35. The method of claim 1, wherein the PAR1 antagonist down-regulates the cellular level of PAR1.

36. The method of claim 35, wherein the PAR1 antagonist is selected from the group consisting of a siRNA, a miRNA, a shRNA, an antisense nucleic acid, and a cDNA.

37. The method of claim 1, wherein the PAR1 antagonist is delivered locally to the heart of the subject.

38. The method of claim 1, wherein the subject is a subject in whom an infarct has occurred and wherein the administration of the therapeutically effective amount of the PAR1 antagonist reduces the size or extent of infarct in the subject.

39. The method of claim 1 further comprising administering to the subject a therapeutically effective amount of a second therapeutic agent for cardiac dysfunction.

40. The method of claim 39 wherein the second therapeutic agent for cardiac dysfunction is an antagonist of protease activated receptor 2 (PAR2).

41. The method of claim 40 wherein the antagonist of PAR2 is selected from the group consisting of FSLLRY—NH₂ (SEQ ID NO: 9); LSIGRL (SEQ ID NO: 10); N¹-3-methylbutyryl-N⁴-6-aminohexanoyl-piperazine; and an antibody specifically binding PAR2.

42. The method of claim 39 wherein the second therapeutic agent for cardiac dysfunction is a monoclonal antibody specifically binding to TF that specifically blocks signaling activity of TF without substantially interfering with coagulation activity of TF.

43. The method of claim 42 wherein the monoclonal antibody specifically binding to TF is selected from the group consisting of:
(a) monoclonal antibody mAb 10H10;
(b) a monoclonal antibody that has complementarity-determining regions that are identical to those of mAb10H10; and
(c) a monoclonal antibody that specifically binds the same antigen bound by mAb 10H10 such that the antibody has an affinity for the antigen that is at least 80% as great as that of mAb 10H10 as measured by the reciprocal of the dissociation constant for the antibody-antigen complex.

44. The method of claim 39 wherein the second therapeutic agent is selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACE/NEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIa inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibitors, renin inhibitors, inhibitors of adenosine diphosphate (“ADP”)-induced platelet aggregation, and NHE-1 inhibitors.

45. A method for treating or preventing hypertrophy in a cardiomyocyte cell or proliferation of a cardiac fibroblast, comprising contacting the cell with an antagonist of protease activated receptor 1 (PAR1), thereby treating or preventing hypertrophy in the cardiomyocyte cell.

46. The method of claim 45, wherein the cardiomyocyte cell or cardiac fibroblast is present in a subject having undergone myocardial injury.

47. The method of claim 46, wherein the myocardial injury is myocardial infarction or cardiac ischemia/reperfusion.

48. The method of claim 45, wherein the PAR1 antagonist is a PAR1-selective antagonist.

49. The method of claim 64, wherein the PAR1 selective antagonist has a binding affinity for PAR1 that is at least 5-fold stronger than the binding affinity of the PAR1 selective antagonist for any of PAR2, PAR3, and PAR4.

50. The method of claim 48, wherein the PAR1 selective antagonist binds to PAR1 with an IC₅₀ that is less than 1 μM.

51. The method of claim 48, wherein the PAR1 selective antagonist binds to all of PAR2, PAR3, and PAR4 with an IC₅₀ that is at least 5 μM.

52. The method of claim 45, wherein the PAR1 antagonist is selected from the group consisting of a peptide, a peptidomimetic, a small molecule organic compound, a pepducin, a polynucleotide, and an antibody.

53. The method of claim 72, wherein the PAR1 antagonist down-regulates the cellular level of PAR1.

54. The method of claim 53, wherein the PAR1 antagonist is selected from the group consisting of a siRNA, a miRNA, a shRNA, an antisense nucleic acid, and a cDNA.

55. A pharmaceutical composition for the treatment or prevention of a cardiovascular dysfunction in a subject, comprising:
(a) a therapeutically effective dose of a PAR1 antagonist; and
(b) a pharmaceutically acceptable carrier, diluent or excipient in unit dosage form.

56. The pharmaceutical composition of claim 55 wherein the PAR1 antagonist is a PAR1-selective antagonist.

57. The pharmaceutical composition of claim 56, wherein the PAR1 selective antagonist has a binding affinity for PAR1 that is at least 5-fold stronger than the binding affinity of the PAR1 selective antagonist for any of PAR2, PAR3, and PAR4.

58. The pharmaceutical composition of claim 55, wherein the PAR1 selective antagonist binds to PAR1 with an IC₅₀ that is less than 1 μM.

59. The pharmaceutical composition of claim 56, wherein the PAR1 selective antagonist binds to all of PAR2, PAR3, and PAR4 with an IC₅₀ that is at least 5 μM.

60. The pharmaceutical composition of claim 55, wherein the PAR1 antagonist is selected from the group consisting of a peptide, a peptidomimetic, a small molecule organic compound, a pepducin, a polynucleotide, and an antibody.

61. The pharmaceutical composition of claim 60 wherein the PAR1 antagonist is a peptide of the sequence X₁-F(f)F(Gn)LR-NH₂, where X₁ is selected from the group consisting of (2-thiophene)acetyl, N-acetyl-2-aminobenzoyl, 2-oxo-(2-thiophene)acetyl, (3-thiophene)acetyl, phenylacetyl, (2-thiophene)sulfonyl, (3-fluorophenyl)acetyl, (4-fluorophenyl)acetyl, 3-pyridylacetyl, (2-fluorophenyl)acetyl, (3-indole)acetyl, cyclopentylacetyl, 2-oxo(3-indole)acetyl, 3-indoloyl, (3-chlorophenyl)acetyl, N-acetyl-4-aminobutyryl, 2-thiopheneoyl, 3-thiopheneoyl, 3-furanoyl, 2-indoloyl, 4-chlorobenzoyl, trans-cinnamoyl, 3-phenyl-2-propynoyl, p-fluoro-trans-cinnamoyl, p-chloro-trans-cinnamoyl, p-methyl-trans-cinnamoyl, p-methoxy-trans-cinnamoyl, 4-biphenyloyl, m-chloro-trans-cinnamoyl, 3-phenylpropionyl, phenoxyacetyl, 1-napthtylacetyl, 3-(2-thiophene)-trans-acryloyl, (±)-trans-3-phenylcyclopropanoyl, 3-coumarinyl, 4-phenylbutyryl, p-amino-trans-cinnamoyl, 2-naphthylacetyl, p-hydroxy-trans-cinnamoyl, (thiophenoxy)acetyl, trans-2-trans-4-hexadienoyl, trans-2-octenoyl, α-fluorocinnamoyl, α-methylcinnamoyl, and α-phenylcinnamoyl, F(f) is a substituted phenylalanine residue with a fluoro group at the para position of the phenyl moiety of the phenylalanine, and F(Gn) is a substituted phenylalanine residue with a guanidino group at the para position of the phenyl moiety of the phenylalanine.

62. The pharmaceutical composition of claim 60 wherein the PAR1 antagonist is a peptide of the sequence trans-cinnamoyl-F(f)-F(Gn)-L-X₂, in which X₂ is selected from the group consisting of Orn-NH₂, Orn(acetyl)-NH₂, Arg-Orn-NH₂, Arg-Orn(N⁵-acetyl)-NH₂, Arg-Arg-NH₂, and Arg-Orn(N⁵-propionyl)-NH₂.

63. The pharmaceutical composition of claim 60 wherein the PAR1 antagonist is selected from the group consisting of N-[3-methyl-1-S[[2-S-[(methyl)aminocarbonyl]-1-pyrrolidinyl]carbonyl]butyl-D-alanine and N-[3-methyl-S-(1-pyrrolidinylcarbonyl)butyl-D-alanine ethyl ester hydrochloride.

64. The pharmaceutical composition of claim 60 wherein the PAR1 antagonist is a PAR1 antagonist of Formula (II).

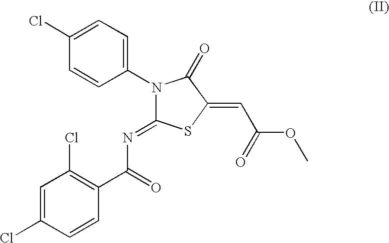

65. The pharmaceutical composition of claim 60 wherein the PAR1 antagonist is a trisubstituted urea with a structure selected from the group of:
(a) trisubstituted ureas of Formula (III), wherein R can be t-butyl-, NH₂CMe₂-, 2,4-difluorophenyl-, 3-pyridyl, 4-(SO₂NH₂)phenyl- and 4-benzimidazole-;
(b) trisubstituted ureas that are derivatives of Formula (III), wherein the 2-propyl substituent on the nitrogen atom of the central urea moiety of Formula (III) is replaced with a substituent selected from the group consisting of n-propyl, cyclobutyl, i-propyl, (±)2-pentyl, (S)sec-butyl, or (R)sec-butyl; and
(c) trisubstituted ureas of Formula (IV), wherein: X is selected from the group consisting of CH₂, S, or SO; R is selected from the group consisting of hydrogen, ethyl, or methyl; the stereochemistry at the 2, 2′, and 5 is selected from the group consisting of S,S; S,R; R,S; R,R; S,S-rac; S,S,S; and S,S; and n is 1 or 2.

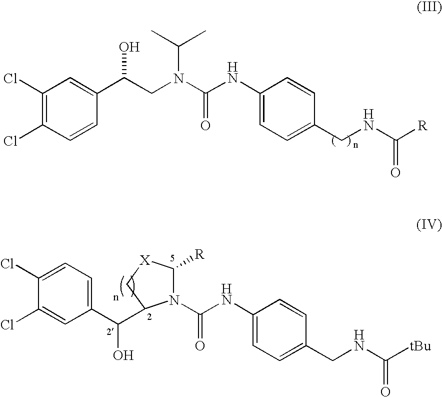

66. The pharmaceutical composition of claim 60, wherein the PAR1 antagonist is selected from the group consisting of:
(a) a compound of Formula (V); and
(b) derivatives of the compound of Formula (V) that have the structure of Formula (VI) wherein R is selected from the group consisting of 3-methyl, 4-methyl, 3-methoxy, 4-methoxy, or 3,5-difluoro.

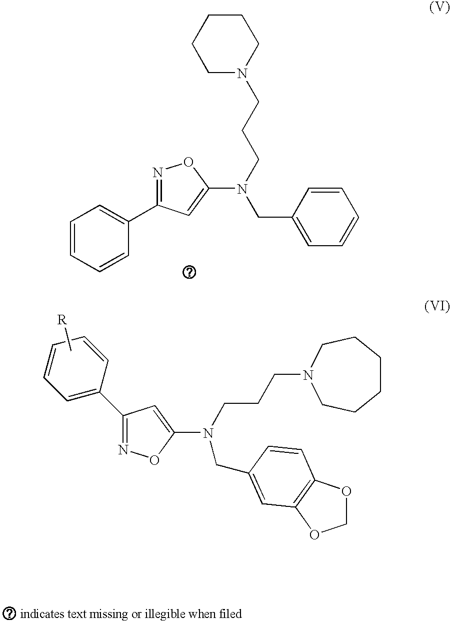

67. The pharmaceutical composition of claim 60 wherein the PAR1 antagonist is a derivative of a 1,3-dihydrobenzoimidazolamine analogue of Formula (VII).

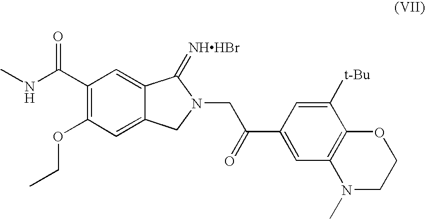

68. The pharmaceutical composition of claim 60 wherein the PAR1 antagonist is a pyrroloquinazoline of Formula (VIII).

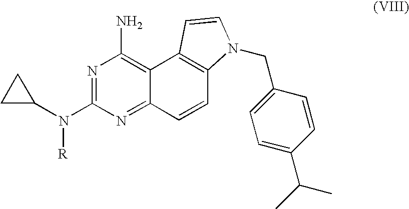

69. The pharmaceutical composition of claim 60 wherein the PAR1 antagonist is (N-3-cyclopropyl-7-{[4-(1-methylethyl)phenyl]methyl}-7H-pyrrolo[3,2-f]quinazoline-1,3-diamine).

70. The pharmaceutical composition of claim 60 wherein the PAR1 antagonist is a benzimidazole derivative of Formula (IX) in which R is selected from the group consisting of hydrogen, methyl, n-butyl, benzyl, and 4-methylbenzyl and R₁ is selected from the group consisting of hydrogen and t-butyl.

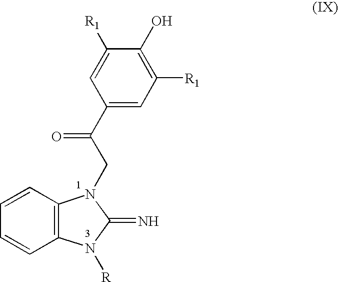

71. The pharmaceutical composition of claim 60 wherein the PAR1 antagonist is a derivative of himbacine.

72. The pharmaceutical composition of claim 71 wherein the derivative of himbacine is a compound of Formula (X).

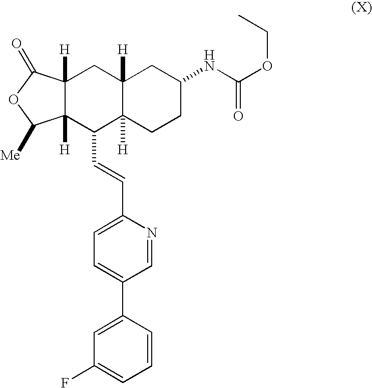

73. The pharmaceutical composition of claim 60 wherein the PAR1 antagonist is an iminopyrrolidine derivative of Formula (XI) wherein: ring A indicates a pyrrolidine ring; ring B indicates a benzene ring or a pyridine ring; R¹⁰¹, R¹⁰², and R¹⁰³ each independently indicate, identically or differently, a hydrogen atom, a halogen atom, a C₁-C₆ alkyl group, or a C₁-C₆ alkoxy-substituted C₁-C₆ alkyl group; R⁵ indicates a hydrogen atom; R⁶ indicates a hydrogen atom, a C₁-C₆ alkyl group, or a C₁-C₆ alkyloxy-carbonyl group; Y¹ indicates a single valence bond or a —CH₂— group; Y² indicates a single valence bond or a —CO— group; Ar¹ indicates a hydrogen atom or a group represented by Formula (XII), wherein, in Formula (XII), R¹⁰, R¹¹R¹², R¹³, and R¹⁴ independently indicate, identically, or differently, a hydrogen atom, a C₁-C₆ alkyl group, or a C₁-C₆ alkoxy group, a morpholinyl group, a piperazinyl group which can be substituted or unsubstituted, or a piperidinyl group which can be substituted or unsubstituted, and R¹¹, R¹², and R¹³ can form a 5-8 membered heterocycle.

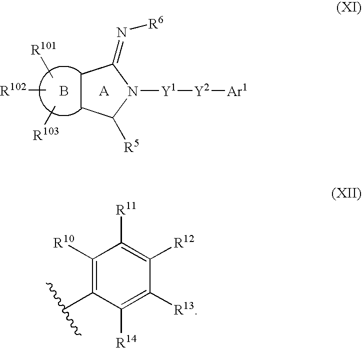

74. The pharmaceutical composition of claim 60 wherein the PAR1 antagonist is a peptide.

75. The pharmaceutical composition of claim 74 wherein the peptide is a peptide of a sequence selected from the group consisting of MSRPACPN (SEQ ID NO: 1) and AFLARAA (SEQ ID NO: 2).

76. The pharmaceutical composition of claim 60 wherein the PAR1 antagonist is eryloside F.

77. The pharmaceutical composition of claim 60 wherein the PAR1 antagonist is a peptidomimetic.

78. The pharmaceutical composition of claim 77 wherein the peptidomimetic is an azole PAR1 antagonist of Formula (XXII) wherein:
(a) A₁ is selected from the group consisting of:
(i) an amino acid residue selected from the group consisting of Sar, Gly, His, His(CH₂ Ph), Ile, Ser, Thr, β-Ala, or Ala;
(ii) a C₂-C₆-acyl group; and
(iii) a C₁-C₈-alkyl group
(b) A₂ is selected from the group consisting of:
(i) an alkyl amino acid residue selected from the group consisting of Cha, Leu, Ile, Asp, and Glu; and
(ii) an amino alkyl amino acid residue selected from the group consisting of Lys, His, Orn, homoArg and Arg;
(c) A₃ is an amino alkyl amino acid residue selected from the group consisting of Lys, His, Orn Arg and homoArg;
(d) A₄ is selected from the group consisting of:
(i) an arylalkyl residue selected from Phe and Tyr; and
(ii) an aralkylamino group selected from the group consisting of a benzylamino group and a phenethylamino group;
(e) R₁ is selected from H or alkyl;
(f) R₂ is a moiety selected from the group consisting of an aryl, substituted aryl, heteroaryl, substituted heteroaryl, aralkyl and substituted aralkyl group;
(g) R₃ is selected from the group consisting of H and alkyl; and
(h) X is selected from the group consisting of S, O, and NR₄, wherein R₄ is selected from the group consisting of H and alkyl;
and the pharmaceutically acceptable salts thereof.

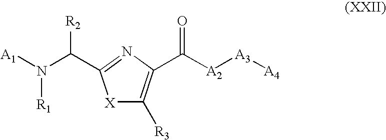

79. The pharmaceutical composition of claim 60 wherein the PAR1 antagonist is a compound of Formula (XXIII) wherein: R¹ and R³ are amide linkages, N-alkylamide linkages, or isosteric replacements of such linkages; R² is either a neutral amino acid side chain or a hydrophobic radical; R⁴ is a hydrophobic radical; R⁵ is Co, CH², or SO; X is either: (1) a moiety of Formula (XXIV) in which R⁶ and R⁷ are the same or different and are hydrogen, alkyl, (cycloalkyl)alkyl, alkoxyalkyl, alkylthioalkyl, or aralkyl; or (2) a hydrophobic residue containing at least one aryl moiety; and R⁸ is a hydrophobic radical or is a hydrophobic residue; Y is alkoxy, hydroxyl, amino, alkylamino, or dialkylamino, in which any of the alkyl groups can be substituted with a basic moiety; Z is an amino acid or peptide residue; and m is 0 or 1.

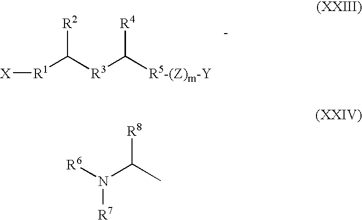

80. The pharmaceutical composition of claim 77 wherein the PAR1 antagonist is a peptidomimetic selected from the group consisting of (αS)—N-[(1S)-3-amino-1-[[(phenylmethyl)amino]carbonyl]propyl]-α-[[[[[1-(2,6-dichlorophenyl)methyl]-3-(1-pyrrolidinylmethyl)-1H-indol-6-yl]amino]carbonyl]amino]-3,4-difluorobenzenepropanamide and a derivative thereof in which the substituted indole ring is replaced by an identically substituted indazole ring.

81. The pharmaceutical composition of claim 60 wherein the PAR1 antagonist is a pepducin.

82. The pharmaceutical composition of claim 81 wherein the pepducin is a pepducin comprising: (1) a first domain that includes either extracellular or intracellular portions of PAR1, the first domain not comprising a native extracellular ligand of PAR1; and (2) at least a second domain, attached to the first domain, wherein the second domain is a hydrophobic moiety which is either naturally or non-naturally occurring.

83. The pharmaceutical composition of claim 81 wherein the pepducin is selected from the group consisting of pal-RCLSSSAVANRS (SEQ ID NO: 3) and pal-KKSRALF (SEQ ID NO: 4).

84. The pharmaceutical composition of claim 55, wherein the PAR1 antagonist down-regulates the cellular level of PAR1.

85. The pharmaceutical composition of claim 84, wherein the PAR1 antagonist is selected from the group consisting of a siRNA, a miRNA, a shRNA, an antisense nucleic acid, and a cDNA.

86. The pharmaceutical composition of claim 55 wherein the pharmaceutical composition further comprises a therapeutically effective amount of a second therapeutic agent for cardiac dysfunction.

87. The pharmaceutical composition of claim 86 wherein the second therapeutic agent for cardiac dysfunction is an antagonist of protease activated receptor 2 (PAR2).

88. The pharmaceutical composition of claim 87 wherein the antagonist of PAR2 is selected from the group consisting of FSLLRY—NH₂ (SEQ ID NO: 9); LSIGRL (SEQ ID NO: 10); N¹-3-methylbutyryl-N⁴-6-aminohexanoyl-piperazine; and an antibody specifically binding PAR2.

89. The pharmaceutical composition of claim 86 wherein the second therapeutic agent for cardiac dysfunction is a monoclonal antibody specifically binding to TF that specifically blocks signaling activity of TF without substantially interfering with coagulation activity of TF.

90. The pharmaceutical composition of claim 89 wherein the monoclonal antibody specifically binding to TF is selected from the group consisting of:
(a) monoclonal antibody mAb 10H10;
(b) a monoclonal antibody that has complementarity-determining regions that are identical to those of mAb10H10; and
(c) a monoclonal antibody that specifically binds the same antigen bound by mAb 10H10 such that the antibody has an affinity for the antigen that is at least 80% as great as that of mAb 10H10 as measured by the reciprocal of the dissociation constant for the antibody-antigen complex.

91. The pharmaceutical composition of claim 86 wherein the second therapeutic agent is selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACE/NEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIa inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibitors, renin inhibitors, inhibitors of adenosine diphosphate (“ADP”)-induced platelet aggregation, and NHE-1 inhibitors.
